SEQUENCE LISTING (147 sequences)

SEQ ID NO: 1 - DNA, 32 bp, Artificial Sequence
  Synthetic construct Forward primer BFR2 FORW PmlI
taaacacgtgagcatggaaaaatcactagcgg

SEQ ID NO: 2 - DNA, 31 bp, Artificial Sequence
  Synthetic construct Backward primer BFR2 BACK SacII
tacaccgcggtcaaccaaagatttggatatc

SEQ ID NO: 3 - DNA, 32 bp, Artificial Sequence
  Synthetic construct Forward primer BMH2 FOR W PmlI
taaccacgtgagcatgtcccaaactcgtgaag

SEQ ID NO: 4 - DNA, 31 bp, Artificial Sequence
  Synthetic construct Backward primer BMH2 BACK SacII
tatgccgcggttatttggttggttcaccttg

SEQ ID NO: 5 - DNA, 34 bp, Artificial Sequence
  Synthetic construct Forward primer COG6 FORW PmlI
taagcacgtgagcatggatttcgttgtagactat

SEQ ID NO: 6 - DNA, 32 bp, Artificial Sequence
  Synthetic construct Backward primer COG6 BACK SacII
taagccgcggtcagtgatcaatacctatcaac

SEQ ID NO: 7 - DNA, 33 bp, Artificial Sequence
  Synthetic construct Forward primer COY1 FORW PmlI
tagtcacgtgagcatggatacgtcagtatattc

SEQ ID NO: 8 - DNA, 31 bp, Artificial Sequence
  Synthetic construct Backward primer COY1 BACK SacII
tacaccgcggctatcgatttatgccatgaac

SEQ ID NO: 9 - DNA, 32 bp, Artificial Sequence
  Synthetic construct Forward primer CUP5 FORW PmlI
tatccacgtgagcatgactgaattgtgtcctg

SEQ ID NO: 10 - DNA, 30 bp, Artificial Sequence
  Synthetic construct Backward primer CUP5 BACK SacII
tacaccgcggttaacagacaacatcttgag

SEQ ID NO: 11 - DNA, 42 bp, Artificial Sequence
  Synthetic construct Forward primer ERO1 FORW SfiI
tataggcccagccggccacgatgagattaagaaccgccattg

SEQ ID NO: 12 - DNA, 33 bp, Artificial Sequence
  Synthetic construct Backward primer ERO1 BACK SacII
tgtcccgcggttattgtatatctagcttatagg

SEQ ID NO: 13 - DNA, 39 bp, Artificial Sequence
  Synthetic construct Forward primer IMH1 FORW SfiI
tataggcccagccggccacgatgttcaaacagctgtcac

SEQ ID NO: 14 - DNA, 31 bp, Artificial Sequence
  Synthetic construct Backward primer IMH1 BACK SacII
tagaccgcggttacttcagagacataaccag

SEQ ID NO: 15 - DNA, 42 bp, Artificial Sequence
  Synthetic construct Forward primer KIN2 FORW SfiI
tcaaggcccagccggccacgatgcctaatccgaatacagcag

SEQ ID NO: 16 - DNA, 38 bp, Artificial Sequence
  Synthetic construct Backward primer KIN2 BACK SacII
tctgccgcggctataggtttaattcttttaaaatatac

SEQ ID NO: 17 - DNA, 34 bp, Artificial Sequence
  Synthetic construct Forward primer KAR2 FORW PmlI
taagcacgtgacgatgtttttcaacagactaagc

SEQ ID NO: 18 - DNA, 29 bp, Artificial Sequence
  Synthetic construct Backward primer KAR2 BACK SacII
tatgccgcggctacaattcgtcgtgttcg

SEQ ID NO: 19 - DNA, 33 bp, Artificial Sequence
  Synthetic construct Forward primer PDI1 FORW EcoRI
cgccgaattcacgatgaagttttctgctggtgc

SEQ ID NO: 20 - DNA, 31 bp, Artificial Sequence
  Synthetic construct Backward primer PDI1 BACK XhoI
cctcctcgagttacaattcatcgtgaatggc

SEQ ID NO: 21 - DNA, 40 bp, Artificial Sequence
  Synthetic construct Forward primer SEC31 FORW SfiI
tataggcccagccggccacgatggtcaaacttgctgagtt

SEQ ID NO: 22 - DNA, 31 bp, Artificial Sequence
  Synthetic construct Backward primer SEC31 BACK SacII
tatgccgcggttaattcaaagtcgcttcagc

SEQ ID NO: 23 - DNA, 35 bp, Artificial Sequence
  Synthetic construct Forward primer SSA4 FORW PmlI
tatgcacgtgacgatgtcaaaagctgttggtattg

SEQ ID NO: 24 - DNA, 30 bp, Artificial Sequence
  Synthetic construct Backward primer SSA4 BACK SacII
tatcccgcggctaatcaacctcttcaaccg

SEQ ID NO: 25 - DNA, 35 bp, Artificial Sequence
  Synthetic construct Forward primer SSO2 FORW PmlI
tacacacgtgacgatgagcaacgctaatccttatg

SEQ ID NO: 26 - DNA, 32 bp, Artificial Sequence
  Synthetic construct Backward primer SSO2 BACK SacII
tatgccgcggttactttcttgtttccacaacg

SEQ ID NO: 27 - DNA, 35 bp, Artificial Sequence
  Synthetic construct Forward primer SSE1 FORW PmlI
tagacacgtgacgatgagtactccatttggtttag

SEQ ID NO: 28 - DNA, 31 bp, Artificial Sequence
  Synthetic construct Backward primer SSE1 BACK SacII
tatcccgcggttagtccatgtcaacatcacc

SEQ ID NO: 29 - DNA, 42 bp, Artificial Sequence
  Synthetic construct Forward primer HAC1 FORW SfiI
gcaaggcccagccggccacgatggaaatgactgattttgaac

SEQ ID NO: 30 - DNA, 19 bp, Artificial Sequence
  Synthetic construct Backward primer HAC BACK1
tggtcatcgtaatcacggc

SEQ ID NO: 31 - DNA, 88 bp, Artificial Sequence
  Synthetic construct Backward primer HAC BACK2 SacII
cctcccgcggtcatgaagtgatgaagaaatcattcaattcaaatgaattcaaacctgactgcgcttctggattacgccaattgtcaag

SEQ ID NO: 32 - DNA, 822 bp, Saccharomyces cerevisiae
atgtcccaaactcgtgaagattctgtttacctagctaaattagctgaacaagccgaacgttatgaagaaatggtcgaaaacatgaaggccgttgcttcatcaggtcaagagttatctgtcgaagaacggaatctattgtcggttgcttacaagaacgtcatcggtgctcgccgtgcttcatggagaatagtttcttcgatcgaacaaaaagaagaatcaaaggagaaatctgaacatcaagttgaattaatccgttcttaccgttctaaaattgaaactgaattgaccaaaatctctgacgacattttatctgtgttagattctcatttaatcccttctgctactactggtgagtctaaagtattttactataagatgaagggtgactaccaccgttatttagctgaattttccagcggagatgcaagagaaaaggcaaccaactcctctttggaggcttataaaaccgcttccgaaatcgccacaactgaattgcctccaactcacccaattcgtttaggtctagctttgaatttctccgtcttctattacgaaattcaaaactctcctgataaggcttgccacttggccaaacaagcctttgatgatgctattgctgagttagatactttatctgaagaatcatacaaggatagcactttgatcatgcaattattaagggacaacttgaccttatggacctctgatatttctgaatctggtcaagaagatcaacaacaacaacaacaacagcaacagcaacagcaacaacagcaacaacaagctccagctgaacaaactcaaggtgaaccaaccaaataa

SEQ ID NO: 33 - DNA, 1605 bp, Saccharomyces cerevisiae
atggaaaaatcactagcggatcaaatttccgatatcgccattaaaccggtcaataaagacttcgatattgaagatgaggaaaatgcatctttatttcaacacaatgaaaaaaatggagaaagtgatttaagcgactatggaaatagcaacacagaagaaaccaagaaggcgcactatttggaggtggaaaagtctaagttaagagcagaaaaaggtttagaactaaacgatccaaaatatacaggtgttaaaggttcaagacaagcattatatgaagaagtttccgagaatgaggacgaagaagaagaagaagaagaggaagaagaaaaagaggaagatgctctttcattcaggacagattctgaagatgaagaagtagagattgatgaagaagaatcagacgcggacggcggtgaaacggaggaggctcaacagaaaaggcatgcactatcgaaactaattcaacaagagactaaacaagctattaacaaactgtctcaatcagttcaaagagatgcttcgaagggttattccattttacaacagacaaaattatttgacaacatcattgatttgagaataaaactacaaaaagctgtaattgcagcaaataagctcccattaactacagagtcctgggaagaggctaaaatggatgattcagaggaaacaaagcgtttgctgaaggaaaacgaaaaactgttcaataatttattcaatcggttgataaatttcagaataaaattccaacttggcgatcatatcactcaaaatgaagaggtggcgaagcataaattgtccaaaaaaagatctctcaaagagctttaccaagaaactaatagcttagactcagaactaaaagagtacaggactgccgtattaaacaagtggtctaccaaagtttcttctgcatcaggtaacgctgctttatcatctaacaaattcaaagctatcaacttacctgcagatgtacaagtcgaaaaccaattatccgatatgtcccgtttgatgaaaagaacaaagttgaacaggagaaacataacgcctttgtatttccaaaaagactgtgctaatggcaggctaccagaattgatttctcccgttgtcaaagatagtgttgatgacaatgagaattcggatgatgggcttgatatcccgaaaaactatgacccaagaagaaaggataacaatgccattgacattaccgaaaacccatatgtttttgatgacgaagatttttaccgtgttttactaaacgatttaattgacaaaaagatttccaacgctcacaattctgaaagtgcagcaattacaatcacctcaactaatgctcgttcgaacaacaagctaaagaagaatatcgatactaaggcttccaagggtaggaaattgaactactcagttcaagatccaattgcgaattatgaagcccccatcacatccggatacaaatggtcagacgaccaaatcgatgaattctttgcgggattgttaggtcaacgagtgaactttaatgaaaatgaggatgaggaacaacatgccagaatagaaaatgacgaagaattagaggctgttaaaaacgatgatatccaaatctttggttga

SEQ ID NO: 34 - DNA, 2520 bp, Saccharomyces cerevisiae
atggatttcgttgtagactatcagacctacgcaatggcggatactgccacgccagaattaccagaacctgagccaagactaaacttaacctcagatgcacagtcacagcccaccggtaaactagatctacagtttaagttgcccgaccttcaacgttattccaataataatgcaactttgccagtagataatgatggtgctggttcgaaagacctacataagaaaatgacacattacgcaatgtcttccattgataaaatacagctttcaaatccaagcaaacaattagggcaaaattcccaggatgaaaaactatcgcagcaagaatctcaaaatttcacgaattacgagccaaaaaaccttgatttatcaaaattagtatccccgtcaagtggttccaacaaaaataccacaaatttggttctttcgaataaactatccaagatattgaacaattacacattgattaactatcaggccacagtccaactaagaaaatccctaaaggttctagaagagaataaagagagattgtcccttgatgaacaaaagctcatgaatcctgaatatgtaggtactttggcaagaagagcattgaggactgatttggaatctcaactgctaaaggaacatattacggtacttgaggaattcaaacctatcattagaaggattaaacgattatcttcttccgtcgaaaaaatacaaagaacgagcgaaaaattactaagtaatgagacaaatgaggttccaacaaataacgtggtacttcaggaaatagatcaataccgtttaaaggcagagcagttgaagctgaaaaaaaaaatactgttatctataagggataggtttactttgaatcaggtagaggacgatgtaatcaccaatggtactatagacaacatctttttcgaggtagtaaagaaagtaatcaatattaaagatgaatcaagtttcttgctgacgcttcctaatttgaatgctggaaatgctttgataatgggagttaatgaaattttagaaaagacaaacaaaaaaatcttcaattatttgatcgattttttatatagttttgaatcctcttcaaatttattaaatgaccatggtactactgaacaagaaagcttaaacatttttcggaagagtctggtcttcctgtcaagtgatctagaattatttaatgagttgttgaaaagagtgaccacactgagatccaagagtattctggatgagtttttgtctcaattcgatatgaattcaactacctctaaacccatcatattatcggcacacgatccaattaggtatattggtgacgtactagcgtccgttcattccatcatcgcaaatgaagctgatttcgtgaagtcactatttgactttcaggatgaagacttaaaagataccccaatttctatacttcaacaaaacaagacattcttgaaaggcatcgacaacaaattgctgaacgatatcatccagtcgctatccaattcgtgtcgtattcgtatcgagcaaatcgtgaggtttgaagaaaatccgatcatcaatttcgagattgtgaggctgctgaaactttacagagttatgttcgagagaaagggaattcaggacgatagttctattattaacaatttaaagtcgttggaagacatttccaaaaacagaattattggatactatgaagactatatgaagcaaacagtcatggcggaaacaaaaaattcttcagatgatttactgccaccagagtggctatcagagtatatgaataaattggtagagttatttgaaatttatgaaaagacacatgctgccgaagatgaggaatcagaagataataaattgctctcatctaagaatttacaaacaattgtagaacaaccaataaaagatgttctgttaaaacaattgcaaacatcttttcctttggcgaaaaaaaatgaaaaagaaaaggcatcattgctaactatagagataaactgtttcgatttaattaaatctagacttcaaccttttgagggattgtttgcacaagatgatgacagccggaaaatcaccatctgggtttgtgataaactgaaggaatatactaagcaaatgctaactttacaaataaaattcctatttgagaatacaggtttagacctttacagcaatttggtcaatatgatttttcctgtggactcagtaaaggatgaattggattatgatatgtacttagccctgagggataattcattgatggaattagacatggtcagaaaaaatgtgcatgataagttgaactattatctacctcaggcgttaacagatgttcaaggtaatttactatttaaattaacgtcaccaatgatagctgatgaaatatgcgatgaatgtttcaagaagttgtcgctattttataatatcttcaggaaactgttgattcatttgtatccgaacaagaaggatcaggtattcgaaattttaaatttttccactgatgaatttgacatgttgataggtattgatcactga

SEQ ID NO: 35 - DNA, 2039 bp, Saccharomyces cerevisiae
atggatacgtcagtatattctcatgcattggatatttgggccaaggcagatttaacgaatcttcaaagagaattggatgctgatgttatagagattaaggataaagaaaccctgtccttgaattcaagaaagtcattagccactgagactaaaaaatttaaaaaactcgaacctgaggaaaaattgaacaatgtgaataaaataattaagcagtaccaacgtgaaattgataatttgacacagagatcaaaattctctgaaaaggttctttttgacgtatacgaaaagctttcagaggctcctgatccacagccgctactacaaagttcgttggaaaaattgggcaaaattgatgactcgaaggaacttaaggaaaaaataagctacctagaagataagctagccaaatatgcagattatgagactttgaaatcaaggttactggacctagagcaaagctctgcaaaaacattggcaaaaagactgactgcgaaaactcaagaaatcaattctacctgggaggaaaaaggaagaaattggaaagagagagaagcagatctattgaaacaattaacaaatgtacaggagcaaaacaaggcactagaggccaaaatatctaaaaatatagatatagaaggtaatggaaacgaagatggtgaccaagaaaacaatcaaaaagaagtatctacaaggattgctgaatataatctagtaacacaggagttggaaactacgcaggctagaatatatcagttagagaaaagaaatgaggaactaagtggtgctcttgcaaaggcaactagtgaagcagaaaaagaaactgagttacatgcaaaggaactaaaacttaaccagctggaaagcgaaaatgcattgttgagtgcatcctatgagcaggaacggaaatcaacatcacatgcaataaatgagttaaaagaacaattaaatagcgttgtggcggaatcggaatcttacaagtcggagctagaaactgttagaagaaaactaaacaattattctgattacaataagataaaagaagaactttctgcattgaaaaaaattgagtttggggtaaacgaagatgattctgataatgacattcgctctgaagacaagaatgataatactttcgaaagttccttactatctgcaaataagaagctccaggctactttggcggaataccgctcaaaaagtacggctcaagaggaagaacgaaacgaattgaaaaaatctgtggaccaattgaagcagcaaatagctactctcaaagaagcaaatgaaaaattagagacggacctagaaaaagtagagaacgtcagtcctcacttcaacgagactgcaagtatgatgtctggtgtaacaagacaaatgaacaatcgtacgtcccataaaatgtccccaacgagttctattattggtattccagaagatggggaactttctggaaaccaatcaaccattttaccaatagttactaaacaaagagacagatttcgttcgagaaatatggatctggaaaagcaactaagacaaggaaactcagaaaagggtaagcttaaactagaaatttcgaagctaaaaggcgacaatacgaagctttatgaacggattaggtatctgcaatcctataataataacaacgctcccgttaatcaaagtacagagcgtattgacgtggaatcccaatactcaagggtgtatgatgaatcgttgcatccaatggcaaattttagacagaacgaattaaaccactacaaaaacaagaaattatcagctttagagaagttattttccagttttgcaaaagtcattttacaaaataaaatgacaaggatggtattcctcttttactgtatcggtttacacggactcgtattcatgatgagcatgtatgtgattaatattagcggctacatgacacctgaggttggtatagtacaatcggcaaagtcttcttcaaatctcaacggaggacttgggggagcagaaaaagtagctgcaggcgttggttcagttcatggcataaatcgata

SEQ ID NO: 36 - DNA, 483 bp, Saccharomyces cerevisiae
atgactgaattgtgtcctgtctacgcccctttctttggtgccattggttgtgcctctgcaattatcttcacctcattaggtgctgcttacggtactgctaagtctggtgttggtatctgtgccacttgtgtgttgagaccagacctattattcaagaacattgttcctgttattatggctggtatcattgccatttacggtttagttgtttccgttttggtttgttattcgttgggtcaaaagcaagctttgtacaccggtttcatccaattgggtgccggtctatcagtcggtttgagtggtctagctgctggtttcgctattggtattgtcggtgatgcaggtgttagaggttcctctcaacaaccaagattattcgtcggtatgattttgattttgatttttgctgaagttttgggtctatacggtttgattgttgctttgttgttgaactccagggctactcaagatgttgtctgttaa

SEQ ID NO: 37 - DNA, 2736 bp, Saccharomyces cerevisiae
atgttcaaacagctgtcacaaattggtaagaatcttaccgatgaattagcgaagggcttagccgatgatatgagccctaccccgtcagaacaacaaatcgaagatgataagagtggcttgccaaaagaaatacaagctaaattaagaaaatttgagaaatatgaacaaaaataccctttgctactctccgcatacaaaaatgaaaaattaaagtcagagaagttagaggctgttgaaaagattttagcggaaaatacacccatatctaatattgacgacgcagtggatacgttgccagcttttttccaggatttaaacaacaaaaataacctattgaatgatgagatcaagagattaactaagcagaactcggaaattccagaaagcgcctctagtgaaactctgaaggataaagaagaggaatttttgaaaaaagagcaaaattataaaaatgacatagacgatctaaaaaaaaaaatggaagctttaaacatagaattggatactgtacaaaaagaaaaaaatgatactgtttcaggtttgagagaaaaaatagttgcactggaaaatatactaaaggaagaaagggaggccaaaaaacagaaagaagaagtatctatatccgaactgaaggaagaattggctataaagaaccattctctcgaggacagtcgaatgaagataaccgaattggagcaaaatttgtcttcgaaaagtactataatggaggaaaagtcctcagagttggcagaactaaatattactttaaaagagaaagagcgcaagctgagtgaattggaaaaaaaaatgaaggagttaccgaaggcgatatctcatcaaaatgtaggaaacaataacagaaggaaaaagaatagaaacaagggaaagaaaaataagggaggcataactacgggtgatatcagtgaagaggaaacggtcgataactcaatcaatactgaagaatatgataagcttaaagaaaatttgcaagaattacaagaaaaatataaagattgtgaagattggaagcaaaagtatgaagatatagaagcagaactaaaagatgctaaagaattggaaaactcacagctcgaaaaatcagcaaaggagctggaaacccttaacaccgagttgatcgataccaagaagtcattgaaagaaaaaaattcggagctagaggaggtgagagatatgctgaggactgtaggcaatgagcttgtggacgcaaaagatgagattaaagagtcttcgagtaaacaaaatgaagaagtgaaaaccgttaagctggagctcgatgatttacgccataaaaatgcaacgatgatcgaggcctacgaagctaaaaatactgagttgagaagtaagatagagttattgagcaagaaagtagagcatctgaagaatttatgtacagaaaaggagaaagagcagactacatcgcagaacaaggtagccaaattaaatgaggagatatctcaacttacctacgaaaaatcaaacataacaaaggagcttacttctttaagaacctcttataaacaaaaggagaaaactgtgagttacttggaggaacaagttaaacaatttagtgagcaaaaggacgtggctgaaaaatccacagaacagctgagaaaagatcatgctaaaatttctaacagattagacttattaaaaaaggaaaatgagacactgcataatgatatcgcaaagaattctaattcctacgaggagtatttgaaagaaaatggtaaattatcggaaagattgaatattttgcaagaaaaatacaataccttgcaaaatgtaaaaagtaattcgaatgaacacatagattctatcaaaagacaatgtgaggaactaaatgtcaagttgaaggaatctacaaaaaaaattttatctttagaagatgaactaaatgaatatgctaatattgttcaagacaaaaccagagaagctaacacattgagaaggttagtttcggacagtcagacagatgattcgagcaaacaaaaagagttggagaataaattggcctatttaacggatgaaaagaataaattggaagcagaattagacttacaaacatccagaaaggccactgaattacaagagtggaagcatacagtaactgagctgaaatcggaaatacacgctttaaagcttcgtgaagagggactaaaatcagaggttgacgcattgaaacatgttaacaatgacatcaaaaggaagactcaagccacttcagatgattccgatcagttggaacagatcacatctaatttaaaactctcattgtctaaggctgatgaaaagaattttgagctacagtctgccaatgagaaacttctgaatttaaataacgaacttaacaagaaatttgatcgattactaaaaaattatcgttcattgtcctctcaattgaatgctttaaaggaaagacaatacagtgacaagtcaggaagagttagtaggtctggttctatcggtactctagctaacgcgaatattgattcctcaccagcgaataactctaatccaactaaattagagaagatacgatcatcaagttcattggagttagactctgagaaaaatgaaaaaattgcatatataaaaaatgttttgttgggatttttggagcacaaggaacaacggaaccaattacttcctgtaatttctatgttgttacaactggacagtactgatgaaaaaagactggttatgtctctgaagtaa

SEQ ID NO: 38 - DNA, 3444 bp, Saccharomyces cerevisiae
atgcctaatccgaatacagcagattacttggtgaatccaaatttcaggaccagtaagggcggatctttatcgccgacgccagaagctttcaacgacacgcgagttgctgcaccagccactcttcgcatgatgggcaagcaatctggaccaagaaatgaccagcaacaagcaccactgatgcctcctgcagatatcaaacagggcaaggaacaggcagctcagagacaaaatgatgcatcgaggcctaatggcgccgtggaattaaggcaatttcatagaagatctttgggagattgggagttccttgaaacggttggcgcaggctctatgggtaaagttaaattggtcaagcatcgtcaaacaaaggaaatttgtgtaataaagattgttaatagggcttccaaggcttatctccataaacagcactctttaccttccccaaagaatgagagtgagatattagaaagacaaaagcggttagaaaaagaaattgcgagggataaaaggactgttagggaagcctctttgggccaaatcctttaccatcctcatatctgtcgtttatttgaaatgtgcactatgtcaaaccatttttatatgctttttgaatacgtttccggtggacagctgttagattatattattcagcatggctcattaaaggaacaccatgcgaggaaatttgccagaggtatagctagtgcgctgcaatacttacatgccaataatattgttcatcgagatctgaaaattgagaatataatgatatctagttcaggtgaaattaagatcattgattttggtctttccaacatttttgattataggaaacaattacatacgttttgtggttccttgtactttgcagcaccagaactattaaaagcgcagccatacacaggacctgaggtagatatttggtcgtttggtattgttctttatgtcttggtctgcggtaaagtaccatttgatgatgagaactcaagcattttacatgaaaaaataaaaaaaggtaaagtagactatccttcacacttatccattgaagttatatctttattaaccaggatgattgttgtcgacccattaagaagagcaacattaaagaatgtcgttgagcatccatggatgaacagaggatacgattttaaggctccatcatatgttcctaatcgtgttccattaacccctgaaatgatagatagccaagttctgaaggaaatgtatcgcctagaatttattgacgatattgaagatacaagaagatcattgatccgattagtaactgaaaaggaatacatccaactttcccaagaatactgggacaaattatccaacgccaaggggttgagttcaagtttaaataataactacctaaattcaacggcacaacaaaccttaatacaaaatcatattacaagtaatccatcgcaaagtggttataatgaaccagatagtaattttgaagatcctactttagcatatcatccattactatcaatatatcacttggtttcagaaatggttgcacggaaattagcgaagttgcaaagaaggcaagcattggccctgcaagcgcaagctcagcaaaggcaacaacagcaacaagtagcacttggcactaaggtcgccttaaataataactccccggatattatgaccaaaatgaggagccctcagaaagaagtagtacctaatcctggtatttttcaagtgccggcaattggaacatcgggaacctcaaacaacactaatacctcaaacaaacctccactgcatgtaatggttcctcctaaactaacaataccggaacaagcgcatacttctccaacatctaggaagagttccgacattcatacggaattaaatggtgttttgaaatcaacaccagtccccgtgtctggcgaatatcagcaacgttctgcttcacccgtagtaggtgaacatcaggaaaagaatacaataggcggcatattcagaagaatatcacaaagtggacaatctcagcatcccacacggcaacaggaacctcttccagaaagagaacctccaacatatatgtcaaaatcaaatgaaatttccatcaaagtaccgaaaagccatagtcgtactatatcagattatattcctagcgctagaagatatccatcttacgtgccaaattctgttgatgtaaaacagaaacccgctaaaaacactaccatagcacctcctataaggtcagtatcacaaaagcaaaacagtgatcttccagctttacctcagaacgccgaactaattgttcaaaaacaacggcaaaaactattacaggaaaatctcgacaaattacaaattaatgataatgataacaacaatgtgaacgctgtagtcgatggtatcaataatgataatagtgaccattatctctccgttccgaagggtcgtaagttacatcctagtgcaagggctaaatcggtggggcatgctcgtcgtgaatctttgaaatttactaggccgcctataccagcagcccttccgccatcagatatgacaaacgataacggctttttgggagaggcaaacaaggagagatacaatcctgttagcagtaacttttcgaccgttcctgaagattctaccacatacagtaacgatactaacaatagactgacttcggtgtattctcaggagcttactgagaagcaaattttggaggaagcttcaaaggcaccccccgggtctatgccatcaattgattatccaaagtcaatgtttttgaagggttttttctctgtacaaacaacctcctctaaaccattgcctattgttcgtcacaatatcatatctgttttaacaagaatgaatattgatttcaaagaagtgaaaggcgggttcatatgtgtccaacaaaggccatctattgagactgcagctgtccctgttataaccactactggcgtgggtttggattccggaaaggcgatggatctgcaaaatagtttagacagtcaattatcatccagttaccatagtacagcgtcctcagcatcaagaaatagttcgataaaacgccaaggttcttataagaggggccagaataatataccactaacacctttagcgaccaatacacatcaaagaaattcatctatcccaatgtctccaaactacggaaaccaaagtaatggtacatcaggggaactatcttccatgtcattagattatgttcaacaacaggatgatattttaacaacatcaagagcccaaaatataaataacgtaaatggtcaaacagagcaaaccaatacttctggtataaaagaaaggcctcctattaaatttgagattcacattgtaaaggttcgtatcgtcggcctagcaggtgtacatttcaaaaaggtttctggtaatacgtggctatataaagaattggcatcgtatattttaaaagaattaaacctatag

SEQ ID NO: 39 - DNA, 3822 bp, Saccharomyces cerevisiae
atggtcaaacttgctgagttttctcgaacagccacgtttgcgtggtcacatgataaaattccattattggtctctggtaccgtatctggtacggtggatgctaatttctccactgattcatctctagaattgtggtcattgttggctgctgattcggagaagcctattgcttccttgcaagtggattccaaattcaatgatttggattggtctcataataacaagattattgctggtgctctggataacggtagtttggaattgtactccaccaatgaagcaaacaacgctatcaactccatggccagatttagcaaccattcttcctctgtgaagacggtaaagtttaacgcaaagcaagacaacgttcttgcttcgggtggtaacaacggtgaaatttttatttgggacatgaataaatgcactgaatcgccctccaattatactccattgacaccgggtcaatcgatgtcgtccgttgacgaggtcatttccctagcatggaaccaatctttggcccatgtttttgcatctgccgggtcgtctaatttcgcatctatttgggatttgaaggctaagaaggaagtcattcatctaagttacacttcacctaattcaggtatcaagcaacagctgtccgttgttgaatggcacccaaaaaactccacaagagtggcaacggctactggtagcgataatgatccatctatcctgatctgggatttaagaaacgccaacacaccattgcagactttaaatcaaggccatcaaaagggtattttgtcattagattggtgtcatcaggacgaacatctattattgtccagtggtagagataataccgttcttctatggaaccctgagtcagccgaacaactgtcccaattcccagctcgtggaaactggtgttttaagaccaaatttgcaccagaggctccagacctatttgcttgtgcctcctttgataacaaaattgaggtacagactttgcaaaatctcacaaacactttggatgagcaagaaaccgaaactaagcagcaagaatctgaaacagatttttggaataatgtttcccgagaggaatcaaaagagaagccatctgttttccatttacaagccccaacttggtatggggaaccatctcccgcagctcattgggctttcggtggtaaattggttcaaattactccagatggtaaaggtgtatctataacaaacccaaaaatttcaggcttagaatcaaacactactttgagtgaagcgttgaaaactaaggatttcaaaccattaataaatcaaagactggtcaaagttattgatgacgttaatgaagaagattggaatttattggaaaagttatcaatggacggtactgaggagttcttgaaagaggctcttgcattcgacaacgatgaatcagatgcacaagacgatgccaacaatgagaaagaagacgatggggaagaattctttcaacaaattgaaaccaatttccaacccgagggcgatttctccttgtctggtaatatcgaacaaactatttccaagaacttggtttctggcaacattaagagcgctgtgaaaaattctctagagaatgacttactaatggaggccatggtgatcgcattagattcaaataacgaaagattaaaggaaagtgtcaagaatgcctattttgcgaagtatggatctaaatcatcgctctcgaggatactatactccatttctaagagggaagtagatgatttggttgaaaatttggatgtctctcagtggaagtttatctctaaagcaattcaaaacttatatccaaatgatatcgcccagaggaatgaaatgttgattaaattgggagacaggttaaaggaaaatggtcatagacaagattctttgactttgtacttggctgccggatcattagataaggtggcttcaatttggttatcagaatttccagatttggaggataaattgaagaaagataataagacaatttatgaagctcattccgaatgtctaactgagttcattgaaagattcaccgtattttccaacttcattaatggaagctctaccattaataatgagcaattaattgccaaatttttggaatttatcaacttaactacttccacaggaaatttcgaactagccactgaattcttaaatagtttaccaagtgacaatgaagaggttaaaacagaaaaggcacgtgtcttgattgcttccggcaaatcattaccggcacaaaatcctgcgacagcgacgaccagcaaagccaagtatacaaacgccaagacaaataagaacgttcctgtactaccaactcctggaatgccttctactacttctattcctagtatgcaggcaccattttatggtatgacaccaggcgcctctgcaaatgctctacctccaaagccgtacgttccagcaaccaccactagtgctcctgttcatacagaaggtaaatatgcgccaccaagccaaccttcgatggcgtcaccttttgttaacaaaacaaatagctcgaccagattgaattcttttgctcctccgcctaacccatatgccactgcaacagttcctgcaacgaacgtatctacaacgtcgattccgcaaaacacttttgctcctatacaacctggtatgcctattatgggcgactataatgctcaatctagctctattccttcacaacctccaattaatgctgtatcgggtcaaacgccacatctcaaccgtaaagccaatgatggttggaatgatttgcctttgaaggtcaaagaaaaaccatctcgtgccaaggctgtatctgttgcccctccaaatatcctatcgacaccaactccattaaatggtatccctgcaaatgctgctagtaccatgcctccgccacctctttccagagctccctcttctgtgtcaatggtatcaccacctcctctacacaaaaattctagagtcccatccttggttgcaacttctgagtcaccaagggcatccatatcaaatccatacgctcctcctcaatcatcacaacaattcccaataggtactatttctacagcaaaccaaacgtcaaacaccgctcaggtagcttcatcgaacccctatgctccaccaccacaacaaagagtagcaaccccattatctggaggcgtgcctccagctccgttgccaaaggcctctaatccatatgctccaactgcaaccactcaacccaacggttcctcctatcctccaaccggtccgtatactaataaccataccatgacctctcctcctcccgtttttaacaaacctcccactggcccccctccgattagcatgaagaagagaagcaacaagttagctagtatagaacaaaacccatctcaaggtgctacttatcctccaaccctttccagctcggcctctccattgcagccttctcaaccgccaactttggcttctcaggttaatacctccgctgagaatgtcagtcatgaaattccagctgatcaacaacccattgtcgacttcttgaaagaagaactggctcgcgtaacaccattgaccccaaaggagtactccaaacaattaaaggattgtgataaacgattaaagattcttttctaccatttggaaaagcaggatttattaacccaaccaacaatcgattgtttacatgacctcgtcgcattaatgaaggaaaagaaatacaaagaagctatggtcatccatgctaatatcgctacaaaccatgctcaagagggtggtaactggctgacaggagtgaagaggttgattggcatagctgaagcgactttgaattaa

SEQ ID NO: 40 - DNA, 1929 bp, Saccharomyces cerevisiae
atgtcaaaagctgttggtattgatttaggtacaacctattcatgtgttgctcattttgcaaacgatagggttgaaattatcgctaacgatcaaggtaatagaacgacgccttcttatgtggcttttactgacacagaaaggctaattggtgacgctgcgaagaatcaagctgcgatgaacccacataatacagtattcgatgctaagcgtctgatcggacgtaaattcgatgatccagaagtgacgaacgatgctaagcattacccattcaaagtgattgacaagggaggtaaaccggtagtgcaagtggaatataaaggcgagacaaagacatttactccagaagaaatttcctcaatgatcttgacaaagatgaaggagactgctgagaactttttaggaacagaagtgaaagatgctgtagtaacggttccagcctatttcaacgattcacaaaggcaagcaacaaaagatgccggtacaatcgcgggcttgaacgttcttcgtatcattaatgaacctacagctgccgctattgcgtatgggctggacaagaaatcgcagaaggagcacaacgtcttgatctttgatttaggtggtggtacttttgatgtctctctgctatccatagatgaaggtgtctttgaggttaaggctactgctggtgacactcacttgggtggtgaagatttcgatagtaggctggttaactttctagccgaggagttcaaaagaaaaaataaaaaggatctaacaactaaccaaaggtccctaaggaggttaaggaccgccgctgaaagggccaagagaactctgtcttcgtctgctcagacatctatagaaatagattcattatttgagggtatcgatttctatacttccattacaagggcaagatttgaagaattatgtgctgatttgtttagatctacattggagccagtggaaaaagttttggctgattcaaaattagataagtcacaaattgatgaaattgtacttgttggtggttcaacaagaattccaaaagtacaaaaactggtttctgattttttcaatggtaaagaaccaaaccgttcgattaaccctgatgaggccgtcgcttatggtgctgccgtacaggctgccatcttaacgggtgaccagtcgtcgacgacccaagatttactgttgctggatgttgcaccattatctctaggtattgaaactgcaggtggtattatgacaaagttgatcccaagaaattcgactatcccaacaaaaaaatcggaagtgttttccacctacgctgacaaccaacctggtgtgttgatacaagtttttgagggtgaaaggacaaggacaaaagacaacaatctactgggtaaatttgagttgagcggtattccacccgctccaagaggcgtaccacaaattgaagttacatttgatatcgatgcaaatggtattctgaacgtatctgccgttgaaaaaggtactggtaaatctaacaagattacaattactaacgataagggaagattatcgaaggaagatatcgataaaatggttgctgaggcagaaaagttcaaggccgaagatgaacaagaagctcaacgtgttcaagctaagaatcagctagaatcgtacgcgtttactttgaaaaattctgtgagcgaaaataacttcaaggagaaggtgggtgaagaggatgccaggaaattggaagccgccgcccaagatgctataaattggttagatgcttcgcaagcggcctccaccgaggaatacaaggaaaggcaaaaggaactagaaggtgttgcaaaccccattatgagtaaattttacggagctgcaggtggtgccccaggagcaggcccagttccgggtgctggagcaggccccactggagcaccagacaacggcccaacggttgaagaggttgattag

SEQ ID NO: 41 - DNA, 2082 bp, Saccharomyces cerevisiae
atgagtactccatttggtttagatttaggtaacaataactctgtccttgccgttgctagaaacagaggtatcgacattgtcgttaatgaagtctctaaccgttccaccccatctgttgttggttttggtccaaagaacagatacttgggtgaaactggtaagaacaagcagacttccaacatcaagaacactgtcgccaacttgaaaagaattattggtttggattaccaccatccagatttcgagcaagaatctaagcacttcacctctaagttggttgaattggatgacaagaagactggtgccgaagttagattcgctggtgagaaacatgttttttcagctactcaactagctgccatgttcatcgacaaagtcaaggacaccgtcaagcaggacacaaaggcaaatattaccgatgtttgtattgctgtcccaccttggtacaccgaagaacaacgttacaacattgctgatgctgctagaattgctggtttgaaccctgttagaattgtcaacgacgttactgctgccggtgtttcttacggtatcttcaagactgatttgcctgaaggcgaagaaaagccaagaattgttgcctttgttgatattggtcactcttcctacacctgttctatcatggccttcaagaagggtcaattgaaagtcttaggaactgcctgcgacaagcattttggtggtagggacttcgatttggctataacagaacatttcgccgatgagttcaaaactaaatacaagattgacatcagagaaaatccaaaggcttacaacagaattctaactgctgctgaaaagttgaagaaagttttgtctgctaatactaatgccccattctctgttgaatccgtcatgaacgacgttgatgtttcctctcaattatctcgtgaagaattagaagaattggtcaagccattgttggaacgtgttactgaaccagttaccaaagctttagctcaagccaaattatctgctgaagaagttgattttgttgaaattattggtggtactactcgtatcccaacattgaaacaatccatttctgaagccttcggcaagccattgtccaccactttgaaccaagatgaagccatcgccaagggtgccgcctttatttgcgccattcactctccaactctaagagttagaccattcaagtttgaggatatccatccttactctgtctcttactcttgggacaagcaagttgaggacgaagaccacatggaagttttcccagctggttcatccttcccatctactaaattgatcactttgaaccgtacgggtgacttttcaatggctgctagctacactgacatcacacagttaccaccaaacactccagaacaaatcgctaactgggagatcactggtgttcaattaccagaaggtcaagactctgttcctgttaagttaaagttgagatgcgacccctctggtttacacacaattgaagaggcttacactattgaagatattgaagttgaagaacctattccattaccagaagatgctccagaagatgctgagcaagaatttaagaaggttactaaaactgtaaagaaggatgacttaaccatcgttgcacacacctttggcctagacgctaaaaagttgaatgaattaattgaaaaagaaaatgaaatgcttgctcaagataagctagttgctgagacagaagaccgtaagaacactcttgaagagtacatctacacattgcgtggtaagttggaagaagagtatgctccatttgcttccgatgctgaaaagacgaagttacaaggtatgttaaacaaggccgaagagtggttatacgatgaaggtttcgattccatcaaagctaagtacattgccaaatacgaagaattggcttctctaggtaacattattagaggtagatacttggctaaagaagaagaaaagaagcaagctataagatctaagcaagaagcatcccaaatggctgctatggctgaaaagttggctgctcaaagaaaggcagaagctgaaaagaaggaagaaaagaaggacactgaaggtgatgttgacatggactaa

SEQ ID NO: 42 - DNA, 777 bp, Pichia pastoris
atgtcaagagaagattctgtttatttagcaaaactagctgagcaagctgagcgttatgaggagatggtcgagaacatgaagaccgtcgcctcttccggcttagagttgtctgtcgaagagagaaacttgctttctgttgcatacaaaaacgtaattggagctagaagagcttcttggagaatcgtctcctcaattgaacagaaagaggaagccaagggtaaccaatcacaagtgtctttgatcagagaataccgctccaagattgagaccgaattggccaacatttgtgaggatattttgtctgttttgagtgagcaccttattccttctgccagaactggcgaatccaaggtcttctactttaagatgaagggtgattaccaccgttatttggccgaattcgctgttggtgacaagcgaaaggaagctgctaatttgtcattggaggcttacaagtctgcctctgacgttgctgttacggagctacctccaactcatccaattagattgggtctggctctgaatttctcagtcttctactacgagattctaaactctcctgaccgcgcctgtcatttagccaagcaagctttcgacgatgctattgctgagttagaaaccctatctgaagaatcttacaaagactccactttgattatgcaactgctgcgtgacaacttgactttgtggacctcagacatgtctgaaactggacaagaagagtcatccaatagccaagataagacagaagctgctcccaaagatgaagagtgaata

SEQ ID NO: 43 - DNA, 1500 bp, Pichia pastoris
atggctagaaagacattggctgaaacattggcagaattgtctcaaccagcgtctggagattttgatatagaagaccaagaaggaggagcagtacttgactatggagataatagttcttttggctccgagagtgaagaggataaaagtaaccactatgttaaagttggcaagtcaaggataagagagaacgcagttaaattgggaggacaatacgagggaaaaaagagtagtagagccgatgtttttggagacgaggacgatgaggaggaggacgatgaggatgttgaacattcggaaactgaagatgcactttcggtttcaggatcagagtccgaatcggatgaaaaaaatagtgatcaaagccaaggtgattctgagagtgaagaagaatctaactcaggtgaagatctagactacaagagatcaaaactacagcaacttataagctccgaaaggaaaaccattgtaaaccaattatcaacttccaataaacaagatgcactgaaagggtttgcagtgttgaatcagcagatacagtatgatcaattggttgacctcagaataaaattacagaaaggattagtagcatcgaatggtctacccattaacaaagaatattacgaacagaataaagcaccaaagtcttccaaacacctggataagctacaagataaactatacaatttattggatgtcactttagaactgagaggcaagctattaaacaaaagcaagattgtgagccaagagtttccccctattccaagtaagaaacgtagtttacagcattatttggaggaatcttccaagttggataacatagttaatgaatatagaaggaacgtcctcgttaaatggtctcaaaaagtccaaaatgcttccggagcaactgctttgagctcatccaaattcaaggctattaaccaagatagttcgactcaagtggacaactatttggcagacatggatagattaatcaaaagaaccagactcaacagaagaagcgtagtgccattaggatacaccgagacagaagaagtagtagatgatgatgaattgatcgacaacgataaagataacaatgagaccaaatacttcagcaacattgaccgatctttgaaggaaaacaaatatatctatgatgatgacgatttctatagagttcttctgaacgatctagtcgataagaaagtttctgatacacagaagctgacatctacatcaactgttattacattttcgaaatccaaattgcataaaagttatgaaagaaaagcgactaagggtcgtaagctgaggtatacagttcaagatccattattgaattttgaagcctccaacccacatgcctacaagtggaacgactaccaaattgacgagttttttgcgtcattatttgggcaaaaggtcaacatgaacgaggatgagcataacgaagaggtacaaggtgaatcagaaggagaggacattttgaaggatgatatcaaactgtttggataa

SEQ ID NO: 44 - DNA, 2351 bp, Pichia pastoris
atggactttgtatatgagtactcagatgctacccctagtggcacatttgatgacccattgcctgcagagcccgaaccaccattcaatttgtcaaacttaaactcgtacaaagatgatttgactaaaaaattctccaaaatgagcattctgaaaagtctgaaaaatgacaccaattcagttgacgatgtcgacgactcacaatcgatctccaatgacgggcagagggcttataaatacgccaatcagtctctggatctggttaaccagcacaccactaataaatcaatcagaaccaccagcgatgaacaaccttcggtgtccactgttttgagcaacagactgagcagagtgctcaataatactaattacgacccttcaaccaaggaactactctccattgtggagaagaaaataaaagaagatacggcgcatgaatacgacaaagttactgacccaagttttgttggaaaccttgctagaagaaagttgcgtaacgacattgaacatgatgttgtagatgccaacttcaatttcttgaaacaattgcaacccttaagaaagaccttgggccagattgaaagtgacttgaatgaaatgaacgagctcaacaatcaaatcactgaaaagttgtcctctagagttgaagataccactaggttggataattccatacacgagttgcatgcaacttccaagattatttccatcaaaaagaagcttttgcagaatttccagaaccgctatactctctcccatttcgaagcacatcaattagagtttggtgaaattgacgattcctttctagaaatactgaaaaaagctgagtcaattcatgatgattgttcaattttgttaaccatggagaatgctactgtgggtattaatattatgaacgacatgaaaaagctttccaataatgccatcgacagattgtcgacatttgtcaccaaacatttttctaggttaagttcgtccaacaatacctccgcctccatagaggataaggcattcctgaaaagatctatactcttcatttccgaaagatacccggagcagctctctggaatcaccaaccaaatagtcgaatcaaggtcaaagtctttgcttgacgagttccaaatacaattgaatggttatgcagattcagcatcaagaaacgaaagggatgttaataaaccattgttcctttccgcatatgattcagaaaggtttctcggagatttacttgcttatattcatggcacaattgttaatgaaagagaaaccgtcgaaagcttgttcagtttgcaagatgaggagaaagataatatcgttttgacaacactcgtagagtcaattgtttcaaagaacatcgaatctctagctacccccctgaatttaaagattgaacagatcatcagaaatgagtctaagctgacagcgatccaagctttttatgacctgctctcactttattccatgatgcttgaaaaaactttaggttctaagaatgcccttttgagtacaatcaactctttaaaagtttcggctttgggtaagattcaaagttcaatcaacattaaacttaaaaacatagagcggactgccaatgagagcatgtcatattataatgaagatgaacaactagatggtacaaaccacaactttgtttcagaaactcattacattgaagaaatcacacctgagctagctgtgcctgattggttgatcaatttctatggtgacgtacttcccatctttgataatgaaaaggtgacaaatgccaaagaactgtatgaggatttactcaaatactgttttgaacaaatcattcaacttatcgagaaacaaatagctcagaataaattgaatgatgctagagagatattgattttcaaatcaaactgttacgattttgtttattccaaaattgtgaccctgaacatctttaaggagaaactggatcgattagaggtaatgataaaggaatgcgaatcaaaattgaccgaaattcagtacacttatcttctcaaacaatcagggttatatgatattcacaaccttgtcaacatgatatcctcaactagggaagatttctttgacgtctccgtttatgaaccaattacggagaactcactattcaatggtgacaaattcaaagaaatatcagatcgccttcaagattttcttccaattgcattaattgattaccaagaggagcgattgttgtatctattacctcccacgcttgttaactctatcattcaaaactcctctgtggattttgtcaacttttatttcaaattatcgttgatcgtgaaggaatatttgaaagccagtgaaggatgtctcagatgggatgacatggaggt

SEQ ID NO: 45 - DNA, 1443 bp, Pichia pastoris
  misc_feature (1)..(546): partial nucleotide sequence from the 5′-end of COY1
aaaagttaagtaatgagttggttagctacagatcgataacaagaggacatggttaattcaattcaggaactggaagagaagcttgcgttttctcaaaagcaagtagagcagttacagcagttaaaccaggatttggagaaggagactagtgtggaaaaatgggatgcaatttcaatgatttctgccaggccggacacttcaatacaggacaattcgttgattacaatggtatcacaacaaagagatcgatttaagcaaaggaacaaagatcttgaaaaagacgttagattacaattgaacaaaatttctgagcttcaaagaaaggtccaatcactttcttcagacaataatcaattatacgagagaatcaggtttttgtcatcctatgacagtaataagaatcagtccaaggagagccagtcggaagagtactacaagagaagttacgaagacaaattgcatccgatagaacaatttagcatattgcatcagcctgcacttaattgtgatgtcaatgacgatgtatgtgatgaatatccataatgaaattcataccgttgtcacgtaatcgcgggggtagtgtgcatcgcatcgtattggagacatctgtctgttttcttccctcacatcgaaatacaacttcaccatgactgacgctgacttccaagtagtatttgaagcgtggcaagctgtcgatctacagggtgttaagaagcttgtagatgatgaggcaaaagagattgaaagttcgaagtcttcaagtttggatcaaagaaagcagttgagttttaagacgaaggagttcaagaaattggacgatgagcgtaaattgacacaatggaggtcgttgttgaaggagtatcaaaactacattgatgatttgaccaagggaaataatcgtgttgtacagacattcttggaattgcataaagtagtggtggatttgaaggatcctacaagtactttgagcaaggagcaagagacgaataccgaattacagaaagctgtgaaaaaactttccacagaactgaggcattcagaacaacactgggcttccgagaagaaaggattggaagaaaaatttaacgtacgtaaaagggaaacggaagagaaaagtcttgatcagattaagacagcccaaactgaaatagtccaattgagggatgagctgaagcaaaaatcttcagaaaatgaagagcttcaagtggtgattgaaacccttgatgccaagctaaaaaagaacagccaaggacaaaataatgatgatacatactccaattatgacatgttaaatagagatttggagtccaataaactaaagatccttgaattggaaaggttgaacaactctctaaaggaggaattagcaaagaaagatgacaaagcctaccaggagagggtcaccgaactcgaaaaggagagtgtggagtatctctcttaaag

SEQ ID NO: 46 - DNA, 473 bp, Pichia pastoris
gtcctgtttatgctccattctttggatccattggttgtgctgcggccatcatctttacctgttttggtgccgcctatggtactgctaagtcgggtgtaggtatttgtgccacctgtgtcttgcgtccagacttactgatcaagaatacagtgcctgttattatggctggtatcattgctatttatgggttggtggtgtctgtgttgatctcttcatcgttgcaacagaagcaggctttgtatactggctttatccaattgggtgccggtttatcagttggtctgtcaggtctggctgctggttttgccatcggaattgttggtgatgctggtgtcagaggtactgctcaacagccaagacttttcgtcggtatgattctgattttgatttttgctgaagttttgggtctttacggtctgattgttgctcttctactgaactctagagcttcccaagatgtcacttgttaaagc

SEQ ID NO: 47 - DNA, 3466 bp, Pichia pastoris
atgttctcaaaactttcccagttatcccagaatttaggcgaagagctctctaggattaatgaggaggttgctgcctctagaaggaaccaactaaagaaaagcagggattcggagagggatacaaagttccttaacatcaaaactcccgatcctgaagctctacaacaaccgggtcatgaggtgaatgaaggcgctgaaaccgaaacagatgctactgagtcaaagggccaagtggttccaaacacaaatatacacttcaatgatctgcctatggagattagggcccgcttgaaaaagtttgcgaaatatgagcagaaatatccgttgttgttggacgcttacaaaactgagaaggccaaatctgaaatagttcatgcttttgaatcaactttacaagaagtcactcctttgcagacaattggagaaattgaacaattcaaagactttatcagcaatatgacccaaaaggctaaattaatggatgaagaattgagagccaaaactggcgagttgaatggcctaaaaaacgaagtgacagaaatgaaagagaaattgaaggctgttcaaggtgagatgaaagccaagtctgctttagcagaagaatctgcgatgaaagccgatcaacttagtgtggatcttgaacgagttgtgagtgagctagaaaatttgaaaaaggaaagggaagagattgttacggagcgtgatgaggcaaccaaggaacgtgacgagtcaacaagagaaagagatatcattctagaagaagtcaaatctaataaaaatcaagaacttttggaggaatacaagtctgagttagaagaagcaaagaacgctcttgcattgagaactgaggagattgaaaatctaaacttgaagttggagtctgaaaagtcggcaaagttatcattagagggtgtagcagatgagcgagatggccttaaagcaaagttagaagcgcaaacaacttccttccaggaagaattagaccaactttctcaagaacgggatcgtttgaattctcaactaacaataggagaaaagtcacagatagaaattgagcaagaaaaaaatgagctcaagagtcagtacaattctgagattaagtcatcgcttagtaaactggaatccgtaattaaagaaagaaatgagctacaacagcaattggaatctcaagagtctttaactttcgaagtggataagctctctaaagagagagatgagctaaggatgcagttggatagagaaaaagaaaattctgcaaaggctagcatcacgccccagaactttgaggttaaaactaaagttgaatccaacaagaacattgaagcacctttatctgaagaactcaggcaagtcaccagagaaagagatgagctaaaagctcagttgttgctcattcaaaagaacccaggaccaagtaagaagaccaatgaaggaaaccgtaacttagagcggaacggtgaaaaaaagtcccatgaccaaaatggtgctgacgatgatcttatcgaacggaagggtgaaagcggtacagatgattcaaaggataatcagctgaaactaatagagcagtcaaccactattacgatgttgaatgaagagattgaaaacttgaaagatatgctgcgtgacgtcggagacgatcttgtagtggctaaagacaagctttcacaggtctctgctgttgatgaaaaaaaacagcacgctcttgaaagggaactggaatcctcaaagttgaagcttgctgagattgaaaaggattacaacgacgatagagttgacctcaagaatgaattgaaactcgttaccgaggaaaaggaaaatcttgaacatgagaatgaaacattgagtcaaagcttgaccgagcttgaaaagctgaaacaagaagtcaaggagaaagctcaagccgttcagaattatgaatctaagtattcgacactatcggatgaattatctttagtcctctccaaacgagatgaattggaaaaagacaaggaaagtttcaggctgaaattgaaagatttggaacagaaaaattccgagactgaacaacaagaggaatcgcagagaactggaactgcagaaatggaagaggagttgcagactttgaaaagggagctagaagcaagctcccagcgcattgaggattataagcaaaagcaactagaactagatgatgaaatatctcttgtacgttctcaaaaagatgaattacagaaaacgataactagcctgcaagaagacctggggaaagagaaagagaatgttaagttactccgtgaaaatatcattgccgaagaaaaagccaaaaattcccaaaaactggcagaaaatgtggcgcagctagacaaattcaaaaagcaggagatatcactcaagcttgagattgcaaaccttgaaaatctaaatgctgaaaaaggctcaaagatcaaaagtttggaagagcatatcacttatcttaatacagagaggcaatctaattatgatgagaatcagaaactgatctcccatactaatgagaaatggaagcaggattacacggagttagtcataaaattgaacaagtgtcagtcagagaacaatagacttacaaaagaattgaacgaatacaaagataaactaaaagatatgaacacctcaaagctgaacagtagcgagacaatcgagtcaatcagaagacaatgcgaagaacttaaaatgatgaataatgaatattctttgaagattgaaagtctacatgaagaactaagttcttcgagttcaattttacaggaacgttccagagaaatgaatactatacgtaaactgctagctgatactgagtccaaatgtgacgaaagaatcaaacagttaaaagcaagaattgataggttagaagaagaaaaggagacgactagccatgaaagctctgtccaggcaagaaagctgagtaaaacaatcgaccagttaaagaaaggcaagaatgaattgtcagtgcagctagaacaatgtaagctagagctggaacatctgaaatccgtcccatctagagtggatgttgacaataaaaacggtgcttcaaatgaaaacagtgatgaaaaccaatctgatattgaatctggaattatcgaacagctcagaaactcgctaaagggatatgaagaacaactaaaacaataccaagattccaacgtcttactcaagaaggttaacgaagagcagttgctgaagttcgagagactgcattcaaatttcaagattgtatctaaacaatatagaatgctgaaagatcaaaaggacgaagtcaatacgagaagtagaaacaattcagttataagttcaacgagcgcggggagtgatgaaaatgagagagataaagttgcctatattaagaacgtccttctaggatttttggaacacaaagatcaacgagctatgctttttcctgtagtgaagatgctacttatgctggacgatgatgaagagagaaggt

SEQ ID NO: 48 - DNA, 3324 bp, Pichia pastoris
atggatagagaacagggtattctgccacaggatcccttctccaactcggtgcatgtaccaaagttgagagcttcttctggtggccagccacagaagcctgtaatacaaaattctgctcctgctactgctaggatgcttcgcaatgcaagttcaagtacgtcagcagctttgttgaaagaattaaacacacatgaacactctcaacgtcaacatactccacagaaacaaccatcattggatgccccggcagcattggttccagttgaatctgccacaaaacaattccaccgaacctccattggagactgggaatttagtaatacaattggggcaggctcgatgggtaaggtcaaagtcgccaaacatagagtcactcacgaggtatgtgccatcaaaatagtcattaggtcagccaaaatctggcagagaaatcaccaaaacgatccagaacctgaaactgaagaaaaaagaaagaagctgcgtgatgaatacaagaaggaattggaacgcgatgaacgtactgtcagagaggcagcactaggaaaaataatgtaccacccaaatatttgtcggttgttcgaatgctatacaatgtctaatcactactacatgctttttgaaatagtccagggggtacagttactggattatattgtttctcatggcaaattgaaggaaacacgcgttcgccagtttgccagaagcattgcttctgctttagattactgccattctaataacatcgttcacagagatctgaaaattgaaaacataatgattaacaaacagggtgaaatcaagttgattgactttggcctttccaacatgtatgatagaagaaatctcctgaaaaccttttgcggctccctatactttgcagcaccggagcttttgtcttgccgtccttacattggtcctgaaattgatgtctggtcttttggggttgtattatttgtccttgtttccggtaaggttccctttgatgacgacagcgtgccaaagcttcatgctaaaatcaaaagaggaaaagttgagtatcctgagtttatttcacctttatgtcattcattgctatctcagatgttagtcgttaatccagatcatagagtcactttgaaagctgcaatggagcacccttggatgaccttaggatttgcagggcctccatcaaactatctccctcagcggtcacctattgtattaccgttggatttaagtgtagtaagagagattgcaaatctgggtttaggaaatgaagaacaaattgctcgagatatcacaaacctgatctcgagcagagaatatgaagcgtgtgttgagaggtggaaacttgatcaacagaaagctaatatcaagggctattccgcgcgtgacgattctgctatcatcgccttccacccgttactttcaacgtactacctcgtggatgaaatgaggaagaggaagctagcaaaaggtgctctcaagggacagacctcggtattagacactgtcaaggtgtctccagacattccaaagacaccagctattccccagaaactagaaactacggatgtggaacagccattgcttgccactgtcccacctgcttatacatctccgcatggacagccagctgaactggaagcgatgattgaaccggcacagccattatctagtgctcatcctttcgagatggatatgacgcagcaacaacatgctagcagaaagacccatatcaagcatgctccagaacgacaagatcgtggcggctataatgtacacaagaataactctggtggtcttaactctttattccgaagactcagtggaaaacgaccccataagaatgaggctgaatgggagccttcatctcccccacctcaagttcatccattttcagttaatgatgcggacaggacttcagtacgtggcgtttcaccaattactcaaccagctgctgtgaagaatgtgacctccaataactccaaaaactacctggaccctgttgatgatagtaaattagttcgtcgtgtaggaagtttgagaattaccaacaaagaaaagcaacaagtgacatctgactttccccgactgcccaattttacgattccagagcaaccgcctaagaatgctcccataccgatacatgcccaacctaccactacaggtacaacctttcaatccaatgatcatgaaatcaaaaagaagttacaggcttcgactagtccaaacgaacaacgtgggcctccaacattggctcctagtcaacagagacggctacatcccactgcgagagccaagtcacttggccattctcgcaagcaatcgcttaatttcaaattcggaggaccagcaaacaatcaattacctgcgttgcctactaaagaaaattatgatgtgtttgaagatgcccaaattaccgataacaatttattaaacccagaagggaaatactctgctaatactaacgtgcatatcaaaccaatgacagaatcccaaattttatttgaggcagaacatgctccacctggaactatgccctcagttgagtaccccaggaccttgtttctcaaaggatttttctctgttcagactacatcctcgaagccgttacctgttattcgatacaacattatagcagctctctgcaaacttaacattcaattcactgaagttaacggtgggtttgtttgcgtttacagaaaaactgaaaatttacaaattggggatatcagatctccagttatagagtcaagagtgaccgatgacactgactccgatgttgcaaactcttccaaattgtcatcttcgtcaacagccaataccagagtcaatgttattgaggatgatagttcatcgccgtcctcagcaagattgaaacatcgccgaaagttttctcttggaaacggaatccttaaccatataaggaaacccacgcttgacgggacagaatttgatgactacgatgcaaccgtaaatacccctgttactcctgcacctgcaaatgttcattctcgttcatcgtcttatcataccgagagtgataatgagtccatggagtcgctgcatgatataagaggtggcagtgatatgatcttgaaaaatgttccagaaagaaatgctagacagatagacacagtcaaggaagaggaaacagatgatgatgatcttggtagtatcaacgaaggatcaacacaccgtacacctttgaaatttgaaattcatattgtcaaagtccctctggttggactatatggtgtgaggttcaagaaaattctgggaaatgcttggatttacaaaaggttggcgtcaaagctgctacaagaattgaatttatagttc

SEQ ID NO: 49 - DNA, 3086 bp, Pichia pastoris
  misc_feature (1)..(2215): partial nucleotide sequence from the 5′-end of SEC31
atggtgaaaataagtgaaataaaaagtacttcaacatttgcatggtcgtctgtagactctaatgtcttggctacagggaccttggctggggctgttgacgactcattctctaccacttcgtcattggaactttgggatgtcctgaacacctcagctcccatattccgaaccaatgttggtgcaagatttcatgatcttgcgtggagtaatccaatctctaagtaccagagaggactacttgcaggtgcttttgataatggaacaattcaattgtgggattcctcatcattgctgaatggatcatctgacagtttaatagagctaaagaaacacactgcgcctgttaaaacaatatctttcaatcctacagagtcacagatatttgcatctggtgcttccaatggccaattattcatttgggatataaatcatctttcagagcctatttcaccgggtgcttctactacccctattaatgacataaactccattgcttggaactccaagatacgtcatattttggcctctgctggaacctcaggctacgcatccatttgggatttaaagaccaagaaagaactattgaacttgagttacactgctccatcaggtcaaagagctaacttaagcaccgttgcatggcatcctactaattcgacaagtgtaataacagcttctgattcggacgctgtaccattgataatgacttgggatttaaggaatactaatgtacctgtagctactcttgaaggtcatcaaaagggtgtattgtccctggattggtgttcgtgggactcagaactattactttcttctggaaaggataactctaccctattgtggaatcccatcagaggctctttgttagcggaatacccaaccaccactaattgggccttcaagacccgcttttcttccaagcttcctgacatttttgcaaccagttcatttgatggtaagataacggtgcagaccttacaggatactacacctgcagaggctcaacaagcaaaagctatcaacgatgacgaattctgggcagacctgtccaacagcgataagaaacatcctaattttttacaacgtcaaactccggcctggcttaaagtgccttccagcgtttcatttggatttggtggaaagattgtaaaagtttccaaggcctctgataaccagtcaattgttgtaattgataatttcacaactaacgatacgctggccaagtccacttcccttcttgcaagcaccatcagcacaaacgattaccaaactcttgtcgacgaaaaacttcgtaccgaagcaaataaccacgactggcaattattgaacgatctattaaaagcggatgatgtgaaagattatttcagatttcagattgtggatccttccgtattgaaacatgacaagtctgaacaaaaggttgaaaacggacaagacatatttgaaaacattgagcaaactgatgaagactttttcaacaatcttgaaagggaaaagaattcagtttctgtcaacattccatcatactctccaactgctctcagccagggactaatccaggaggctctagtattggctttaggtgcatctgaatcattacaggctaaagttaggaatgcctattttaaccaaacgcaaaagtcctctctaccaagattgatttacagtgctactgctaacgatgttaatgacctcgttgctaatggtactatctctggttggagggatatagcagctgctatttttgcttactctacggagaaagaagagttttcaaagttcattgtggaactaggtgataggctattagccagttccctttcagatagacgctctgatgctctgctttgcttccttgctggtggtgcgctcaacaaggcgtctacaatttggaatgccgagttgagttctcgtgaagaggttctcaaatctgaaaaccctcagctctcatcttatgaagctcataatattgtgttgactgagtttgttgaaaaaattgccgcattcaagtatgcattaaggatcagcaataagttcagtgggcagggcgttaatacgttgaacaattcattcctagagtttgcttctttggtgtcatctcaagggcaatttgatttggccttgaacttattggagaacttatctaccgaggatgaagacattaaacttgagataaagcgaatctcaacagcatcaggaaaaactctttccagtatcctcccncgctcacccctcccgnccttcccctatcgcatttccaaaggtcttctcgtggaggctcattctccgttccacctcctaatccatacgtaggtagctcagtgaatgggaatggaggagtgcacggaggcgcaccagctatccctgttgccaacaacccttatgctaacaacaatcaaaatgcatcatatggccaagcaaacggtccactaaatggatttgtacctccgccaccaatgcctgagaaaatgggaggactttcctcacagaattaccccaagagagcagcaagtagagcaaatagcactgctggatatgcgccatcactaagatcgcccagtgtgcaacaatttcaaccaccaccacctccggcactagctcaacatgtgcaaccgccaccacctcctgaactagttcagcaggtacctccacccgcgccgtctgtacaacaccaagtatcacaaggatctcaaggatctcaaggtgggccccctgcacaacaacagaccagatttcccagtggagatagatcacatataagtgatgaggctttccctatttatgagtacctgagtaaggagttggaaaatgttaagcctaagattccagaaagatttaccaaacaactcgtagacgctgagaagagattgaatatcttgtttgatcatttgaataacaatgagctgttaaccgctcctacgattacactgttgtctaatctttcaaagtctctagctgaccatgactttaagactgctgaatcgttactgattcaaattactaccattcataacaacgaggcaggaaactggagcgttggtgtgaaacgtcttatccagatgtcctcggctctgagtagctaagaa

SEQ ID NO: 50 - DNA, 1974 bp, Pichia pastoris
atgggtaaatcaattggaattgatttgggtaccacatactcttgtgtggcacattttgctaatgatcgtgttgagatcatagctaacgaccaaggtaacaggacgactccatcgttcgtcgcctttaccgacactgaaagattgattggtgatgctgcaaagaaccaagctgccatgaatccagctaacactgttttcgatgccaaacgtttaatcggtagaaaattcgacgacccggaaactcaggccgatattaagcacttccctttcaaagttatcaacaaggggggaaagcctaatatccaagtcgaatttaagggtgagactaaggttttcagccccgaagagatttcctccatggttctaacaaaaatgaaggatactgctgagcagtatttgggtgagaaaatcaacgatgcagttgtcactgttcctgcttacttcaatgactctcaaagacaagccaccaaggatgctggtttgattgctggtttgaacgttcaaagaatcattaatgagcccaccgctgccgcaattgcttacgggttggacaagaaggatgcaggccacggtgagcacaacattctaatcttcgatctaggtggaggaactttcgatgtttctctactatctattgatgagggtattttcgaagtcaaggccaccgcaggtgacacccacttgggtggtgaggacttcgataacagattagtcaaccactttatcgccgagttcaagagaaagaccaagaaagatctttctacaaaccagagatcccttagaagactaagaaccgcttgtgagcgtgcaaagagaactttgtcttcttctgctcagacctccatcgagattgattctttgttcgagggtatcgacttctacacctcgatcactagagctagattcgaggagctctgtgccgacttgttcagatccaccatcgagcctgttgagagagtcttgaaagactccaagttggacaaatctcaagttcatgagattgttttggttggtggttctaccagaattccaaaggttcagaaattagtttctgactttttcaatggtaaggagccaaacaagtccatcaacccagacgaagccgttgcatatggtgctgctgtccaagcagctattttgtctggagatacttcttccaagacacaagacttgttattgctggatgttgctcctctatctttgggtattgaaaccgctggtggtatcatgaccaagctgatcccaagaaactccacaatcccagccaaaaagtcagaaatcttttcgacatatgctgacaaccaaccaggtgttttgattcaagtctttgaaggtgagagaactagaaccaaggacaacaacctgttgggtaagtttgaactttctggtattcctcctgctccaagaggtgttcctcaaattgaggtcaccttcgatatggatgccaacggtattttgaatgtatctgctgttgagaagggtaccggtaagactcaaaagattactattaccaacgataagggaagattgtccaaggaagacatcgagagaatggtttctgaagctgaaaaattcaaggatgaagacgagaaggaagccgagagagttgctgccaagaatggcttggaatcatatgcttactctctgaagaactctgcagctgaatctggattcaaggacaaggttggagaggatgatcttgccaagttgaacaagtcagttgaagagacaatatcttggttagatgagtcacaatctgcttccacagacgagtacaaggacaggcaaaaggaattggaagaagttgctaacccaataatgagcaagttctatggagctgctggtggagctcctggtggagctcctggtggcttccctggaggtttccctggcggagctggcgcagctggcggtgccccaggtggtgctgccccaggcggagacagcggaccaaccgtggaagaagtcgattaa

SEQ ID NO: 51 - DNA, 2124 bp, Pichia pastoris
atgagtgttccatttggagtagatctaggtaacaacaacactgtgatcggtgttgcccgtaacagaggtattgatattcttgtcaatgaagtctctaatcgtcagacccccagcattgtcggatttggcgctaagtctagagccatcggggaatcaggaaagacccaacagaactctaacttgaagaataccgttgaacatttggtccgtattctcgggcttcctgcagactctcctgactatgaaattgagaagaagttcttcacttcgcccctgattgagaaggacaatgagatcctgtctgaagttaacttccaaggtaagaagactaccttcacacccattcagctggttgccatgtacctgaacaagattaagaacactgccataaaggaaacaaagggaaagttcactgatatctgtcttgctgtccctgtttggttcaccgagaaacagagaagtgctgcttccgatgcttgtaaggttgctggtctgaacccagttagaattgtcaacgacatcacagctgctgcagttggatatggtgtcttcaagactgacctaccagaggatgaacccaagaaggttgcaatcgttgatataggccactctacctattctgttttgattgctgctttcaagaaaggtgagctgaaagtgttaggatctgcttctgacaagcatttcggtggtcgtgatttcgactatgccatcaccaagcactttgcagaggagttcaagagcaaatacaagattgatatcactcaaaatcctaaggcttggtctcgtgtttacactgctgccgaaaggttgaagaaggttttgtccgctaacactacagctccattcaatgttgaatctgttatgaacgacgttgatgtttcttcttcgctgactagagaggagttagaaaagctggtgcaaccattattagaccgtgctcatattcccgttgagcgtgctctggccatggcaggtctcaaggctgaagatgtggacactgttgaggttgtcggaggttgtactcgtgttccaaccttgaaagctactctatctgaagtctttggaaagcccttatctttcactttaaaccaagatgaggcaattgctcgtggtgcagctttcatctgtgcaatgcactcccctacacttagagttcgtccattcaagtttgaggacgttaacccttactctgtgtcatattattgggacaaagatcctgccgctgaggacgatgaccacttagaggtcttcccagtgggtggttctttcccatcaactaaggtgatcacactttaccgttcacaagatttcaacattgaagcccgctacacggacaagaatgcacttccagctggcactcaggagttcattggcaggtggagcatcaagggtgttgttgtcaatgaaggtgaagatactatccagactaagattaagctgagaaatgatccatctggtttccatatcgtcgaatctgcttacacagtcgagaagaagactattcaagagccaatcgaggatccagaagctgatgaagatgcagaacctcagtacaggacagttgagaagctcgtcaaaaagaacgacttggagattactggacagacactccacctaccagatgagctattaaactcttatcttgagacagaggctgccttagaggtccaagacaaacttgttgcagacaccgaggagcgcaagaacgctctggaggagtacatttacgagcttagaggtaagttggaagaccagtacaaggagtttgctagcgaacaggaaaaaaccaagcttacagctaagctagagaaagctgaggaatggctttacgacgaaggttatgattctactaaagctaagtacattgctaaatacgaagagcttgcctccattggaaatgttatccgaggtcgttatcttgccaaagaggaggagaagaaacaagctatccgtgaaaaggaagaatctaagaaggcttctgctatcgctgaaaagatggctgccgagcgtgcttctcgtgaagctgctggttctacaaatgaacaagcccagaagaatgaagaaaacaccaaagatgccgacggtgatgtttctatgaaccaagatgagctagattaaact

SEQ ID NO: 52 - DNA, 37 bp, Artificial Sequence
  Synthetic construct primer pBR322_FOR_NotI
aatagcggccgcgcatctcgggcagcgttgggtcctg

SEQ ID NO: 53 - DNA, 40 bp, Artificial Sequence
  Synthetic construct primer pBR322_BACK_NotI
gattgcggccgcgacgtcaggtggcacttttcggggaaat

SEQ ID NO: 54 - DNA, 30 bp, Artificial Sequence
  Synthetic construct primer puc19ORI no.1-SacI
gatcgagctctgagcaaaaggccagcaaag

SEQ ID NO: 55 - DNA, 29 bp, Artificial Sequence
  Synthetic construct primer puc19ORI no.2-SacI
gaaagagctcccgtagaaaagatcaaagg

SEQ ID NO: 56 - DNA, 30 bp, Artificial Sequence
  Synthetic construct primer ampR no.1 Hind III
gccgaagcttacaataaccctgataaatgc

SEQ ID NO: 57 - DNA, 29 bp, Artificial Sequence
  Synthetic construct primer ampR no.2 Hind III
gccgaagcttaaatcaatctaaagtatat

SEQ ID NO: 58 - DNA, 42 bp, Artificial Sequence
  Synthetic construct primer cyc1TT_neu_FOR_BamH1
caatggatccccttttcctttgtcgatatcatgtaattagtt

SEQ ID NO: 59 - DNA, 34 bp, Artificial Sequence
  Synthetic construct primer cyc1TT no.2-Age I
gtggaccggtagcttgcaaattaaagccttcgag

SEQ ID NO: 60 - DNA, 43 bp, Artificial Sequence
  Synthetic construct primer zeoR_neu_no.1_kpn1
gatcggtacccacacaccatagcttcaaaatgtttctactcct

SEQ ID NO: 61 - DNA, 62 bp, Artificial Sequence
  Synthetic construct primer TEF1_back_no.1
tactatgccgatgattaattgtcaacaccgcccttagattagattgctatgctttctttcta

SEQ ID NO: 62 - DNA, 81 bp, Artificial Sequence
  Synthetic construct primer TEF1_back_no.2_Nco1
ttggccatggtttagttcctcaccttgtcgtattatactatgccgatatactatgccgatgattaattgtcaacaccgccc

SEQ ID NO: 63 - DNA, 43 bp, Artificial Sequence
  Synthetic construct primer Sh ble_FOR_no.1_Nco1
taaaccatggccaagttgaccagtgccgttccggtgctcaccg

SEQ ID NO: 64 - DNA, 70 bp, Artificial Sequence
  Synthetic construct primer Sh ble_back_no.2_aat1
tccgaggcctgggacccgtgggccgccgtcggacgtgtcagtcctgctcctcggccacgaagtgcacgca

SEQ ID NO: 65 - DNA, 62 bp, Artificial Sequence
  Synthetic construct primer cyc1TT_FOR_no.1_aat1
tcccaggcctcggagatccgtcccccttttcctttgtcgatatcatgtaattagttatgtca

SEQ ID NO: 66 - DNA, 45 bp, Artificial Sequence
  Synthetic construct primer cyc1TT_neu_back_Kpn1
acatggtacctgcaaattaaagccttcgagcgtcccaaaaccttc

SEQ ID NO: 67 - DNA, 29 bp, Artificial Sequence
  Synthetic construct primer kanR no.1-Kpn I
ccgaggtaccgacatggaggcccagaata

SEQ ID NO: 68 - DNA, 30 bp, Artificial Sequence
  Synthetic construct primer kanR no.2-Kpn I
ccgaggtaccagtatagcgaccagcattca

SEQ ID NO: 69 - DNA, 45 bp, Artificial Sequence
  Synthetic construct primer 5_AOX TT no.1 HindIII/NotI
gattaagcttgcggccgcagaggatgtcagaatgccatttgcctg

SEQ ID NO: 70 - DNA, 48 bp, Artificial Sequence
  Synthetic construct primer 5_AOX TT no.2 AscI/BamHI
gattggatccggcgcgccgatactcgagaattatggcttaatcaagtg

SEQ ID NO: 71 - DNA, 40 bp, Artificial Sequence
  Synthetic construct primer 3_AOX TT no.3 BamHI
gattggatcctatgattggaagtatgggaatggtgatacc

SEQ ID NO: 72 - DNA, 46 bp, Artificial Sequence
  Synthetic construct primer 3_AOX TT no.4 NotI/EcoR I
taaagaattcgcggccgcagcaacgttgtcactgaagttggcatca

SEQ ID NO: 73 - DNA, 44 bp, Artificial Sequence
  Synthetic construct primer eGFPno.1 Aar I/Sbf I
gatccacctgcaggccatggtgagcaagggcgaggagctgttca

SEQ ID NO: 74 - DNA, 46 bp, Artificial Sequence
  Synthetic construct primer eGFPno.2 SfiI
ggatggccgaggcggccttacttgtacagctcgtccatgccgagag

SEQ ID NO: 75 - DNA, 32 bp, Artificial Sequence
  Synthetic construct primer Paox no.1 Apa I
aaccgggccctctaacatccaaagacgaaagg

SEQ ID NO: 76 - DNA, 36 bp, Artificial Sequence
  Synthetic construct primer Paox no.2 Sbf I
catggcctgcaggtgtcgtttcgaataattagttgt

SEQ ID NO: 77 - DNA, 31 bp, Artificial Sequence
  Synthetic construct primer Pgap no.1 Apa I
aaccgggcccagatcttttttgtagaaatgt

SEQ ID NO: 78 - DNA, 48 bp, Artificial Sequence
  Synthetic construct primer Pgap no.2 Sbf I
catggcctgcaggtgatagttgttcaattgattgaaatagggacaaat

SEQ ID NO: 79 - DNA, 34 bp, Artificial Sequence
  Synthetic construct primer Pgnd1 no.1 Apa I
tatcgggccctatggtagaatcatcaattggaat

SEQ ID NO: 80 - DNA, 43 bp, Artificial Sequence
  Synthetic construct primer Pgnd1 no.2 Sbf I
catggcctgcaggtgatttgtatcagtcttgtttcttttcttt

SEQ ID NO: 81 - DNA, 39 bp, Artificial Sequence
  Synthetic construct primer Pgpm1 no.1 Apa I
tattgggcccgaaagaaggtttatctgactgttgcgcac

SEQ ID NO: 82 - DNA, 39 bp, Artificial Sequence
  Synthetic construct primer Pgpm1 no.2 Sbf I
catggcctgcaggtgtgtttgtttgtgtaattgaaagtt

SEQ ID NO: 83 - DNA, 35 bp, Artificial Sequence
  Synthetic construct primer PHSP90 no.1 Apa I
gactgggcccttcaagatcttttgaggactagaga

SEQ ID NO: 84 - DNA, 45 bp, Artificial Sequence
  Synthetic construct primer PHSP90 no.2 Sbf I
catggcctgcaggtgattgatatttttccaaaattaaaaagttaa

SEQ ID NO: 85 - DNA, 45 bp, Artificial Sequence
  Synthetic construct primer Pkar2 no.1 Apa I
atcagggcccactatcaaagctatcaattgtggaaatggacagca

SEQ ID NO: 86 - DNA, 48 bp, Artificial Sequence
  Synthetic construct primer Pkar2 no.2 Apa I
catggcctgcaggtgtcttgagtgttggaattgaaattaaggaagaag

SEQ ID NO: 87 - DNA, 30 bp, Artificial Sequence
  Synthetic construct primer Pmcm1 no.1 Apa I
gtacgggcccacagctttggcttgaacaat

SEQ ID NO: 88 - DNA, 42 bp, Artificial Sequence
  Synthetic construct primer Pmcm1 no.2 Sbf I
catggcctgcaggtggctaaatgaatgcgggttagtgtttga

SEQ ID NO: 89 - DNA, 37 bp, Artificial Sequence
  Synthetic construct primer PPpet9 no.1 Apa I
agtacgggccctagaaaattcaccactgtcggaaagt

SEQ ID NO: 90 - DNA, 45 bp, Artificial Sequence
  Synthetic construct primer Ppet9 no.2 Sfi I
catggcctgcaggtggaagtcgacgaagaagttagacttgttgtt

SEQ ID NO: 91 - DNA, 40 bp, Artificial Sequence
  Synthetic construct primer Prad2 no.1 Apa I
gtaagggcccgtatagtttgcagacatagtaggagagttt

SEQ ID NO: 92 - DNA, 44 bp, Artificial Sequence
  Synthetic construct primer Prad2 no.2 Sbf I
cattgcctgcaggtgatccttagcccaacctgatggaaaaacgg

SEQ ID NO: 93 - DNA, 30 bp, Artificial Sequence
  Synthetic construct primer Prps2 no.1 Apa I
gtacgggccctcctgagaacggacagcagc

SEQ ID NO: 94 - DNA, 45 bp, Artificial Sequence
  Synthetic construct primer Prps2 no.2 Sbf I
catggcctgcaggtgattaactacactgaaaaagtcggaatgtac

SEQ ID NO: 95 - DNA, 34 bp, Artificial Sequence
  Synthetic construct primer Prps31 no.1 Apa I
gtacgggcccttgtttatagcctataatcgcaga

SEQ ID NO: 96 - DNA, 45 bp, Artificial Sequence
  Synthetic construct primer Prps31 no.2 Sbf I
catggcctgcaggtgtttggcttcgtcggcaatacgtgaatgctt

SEQ ID NO: 97 - DNA, 30 bp, Artificial Sequence
  Synthetic construct primer Pssa1_2 no.1 Apa I
gtaagggcccgttgtatccattcactattt

SEQ ID NO: 98 - DNA, 43 bp, Artificial Sequence
  Synthetic construct primer Pssa1_2 no.2 Sbf I
catggcctgcaggtgaatgtttaactttgtttaatttctatgc

SEQ ID NO: 99 - DNA, 32 bp, Artificial Sequence
  Synthetic construct primer Pthi1 no.1 Apa I
gtaagggcccatcttttcagcttcatcgtcag

SEQ ID NO: 100 - DNA, 42 bp, Artificial Sequence
  Synthetic construct primer Pthi1 no.2 Sbf I
catggcctgcaggtggatgatttattgaagtttccaaagttg

SEQ ID NO: 101 - DNA, 33 bp, Artificial Sequence
  Synthetic construct primer Ptpi no.2 Apa I
gtaagggcccttcaacgagacactcttccgtca

SEQ ID NO: 102 - DNA, 40 bp, Artificial Sequence
  Synthetic construct primer Ptpi no.2 Sbf I
catggcctgcaggtgtgtgtttgtgatagatcttgtatat

SEQ ID NO: 103 - DNA, 32 bp, Artificial Sequence
  Synthetic construct primer Pubi4 no.1 Apa I
agaagggcccagaagattaccataaattgaga

SEQ ID NO: 104 - DNA, 43 bp, Artificial Sequence
  Synthetic construct primer Pubi4 no.2 Sbf I
catggcctgcaggtgaaagcgacaaacgtcacgtgaacaaaag

SEQ ID NO: 105 - DNA, 32 bp, Artificial Sequence
  Synthetic construct primer Peno no.1 Apa I
tatcgggcccaaagagtgagaggaaagtacct

SEQ ID NO: 106 - DNA, 43 bp, Artificial Sequence
  Synthetic construct primer Peno no.2 Sbf I
catggcctgcaggtgttttagatgtagattgttataattgtgt

SEQ ID NO: 107 - DNA, 34 bp, Artificial Sequence
  Synthetic construct primer Prsp7 no.1 Apa I
tatcgggccctttcatccagctctttaaccttat

SEQ ID NO: 108 - DNA, 45 bp, Artificial Sequence
  Synthetic construct primer Prsp7 no.2 Sbf I
catggcctgcaggtgcttgtgatactgctgttaccgtgtgagttt

SEQ ID NO: 109 - DNA, 46 bp, Artificial Sequence
  Synthetic construct primer Prpl1 no.1 Apa I
tatcgggcccataagtcctagaacaccacttgttagtaaaaccggt

SEQ ID NO: 110 - DNA, 45 bp, Artificial Sequence
  Synthetic construct primer Prpl1 no.2 Sbf I
catggcctgcaggtgtttctattaattcgtctccctagcaaaaag

SEQ ID NO: 111 - DNA, 40 bp, Artificial Sequence
  Synthetic construct primer Ptkl no.1 Apa I
tttagggcccgatatcgattccactgctcagagtcttttc

SEQ ID NO: 112 - DNA, 43 bp, Artificial Sequence
  Synthetic construct primer Ptkl no.2 Sbf I
catggcctgcaggtgtgtgtagagtggatgtagaatacaagtc

SEQ ID NO: 113 - DNA, 40 bp, Artificial Sequence
  Synthetic construct primer Ppis no.1 Apa I
aaccgggccctttttcctcttcgttgtgtggtaaactcgg

SEQ ID NO: 114 - DNA, 46 bp, Artificial Sequence
  Synthetic construct primer Ppis no.2 Sbf I
tgatgcctgcaggtggactatctagagacaagtaaatttccatgtt

SEQ ID NO: 115 - DNA, 40 bp, Artificial Sequence
  Synthetic construct primer Pfet3 no.1 Apa I
aaccgggccctttcgtaccaaatggaaaaatcacgtacaa

SEQ ID NO: 116 - DNA, 44 bp, Artificial Sequence
  Synthetic construct primer Pfet3 no.2 Sbf I
taatgcctgcaggtgaaaactagatcctctttggaacaggccgt

SEQ ID NO: 117 - DNA, 36 bp, Artificial Sequence
  Synthetic construct primer Pftr1 no. 1 Apa
aaccgggccctcgagtaacacactactaacttttta

SEQ ID NO: 118 - DNA, 42 bp, Artificial Sequence
  Synthetic construct primer Pftr1 no. 2 Sbf I
taatgcctgcaggtgtttgaaaagaactacaacgaccactga

SEQ ID NO: 119 - DNA, 47 bp, Artificial Sequence
  Synthetic construct primer Pnmt1 no.1 Apa I
aaccgggccctaacatgatatcatgatgtacgtacaaactaggatct

SEQ ID NO: 120 - DNA, 36 bp, Artificial Sequence
  Synthetic construct primer Pnmt1 no.2 Sbf I
taatgcctgcaggtggattggtgattttgatggtca

SEQ ID NO: 121 - DNA, 35 bp, Artificial Sequence
  Synthetic construct primer Ppho8 no.1 Apa I
attagggcccggtataagtatagcacatgttgacg

SEQ ID NO: 122 - DNA, 45 bp, Artificial Sequence
  Synthetic construct primer Ppho8 no.2 Sbf I
taatgcctgcaggtgtgctttgaaattgaaggggagaggacgcta

SEQ ID NO: 123 - DNA, 37 bp, Artificial Sequence
  Synthetic construct primer Pfet3pre no.1 Apa I
agcagggcccttgtggtcctatgaattaaccatttaa

SEQ ID NO: 124 - DNA, 48 bp, Artificial Sequence
  Synthetic construct primer Pfet3pre no.2 Aar I
ctagtcatggcctgcaggtgtcgatggagtgttggcggcagtggttac

SEQ ID NO: 125 - DNA, 1003 bp, Pichia pastoris
caggtagaaaattcaccactgtcggaaagttgtctacttccgtcggttggaaatacgagtctgttgttgagaagttggaggagaagagaaaggctgaggaagctgagtaccaggagaagaagagagcttacacccagagattagacgcagctagtgccgagtttgcccaaaccgaggagggaaagcagttggctgcctttggttactaaatagtaaagtagggtatcttcaagtaatagtatactaaccatctgaaataaccaccgtcctgtagttttttttcgatatcgaagagcctatgctagtactgtggatttgcgctccatccaacatctgtgcgcaaactaaaacttccgagactgacatctaccatcgctagaccctaagtaaaaccaatctcgcgtccgaacttttaaatttcagtccttaaaacttcagagcattggttgtagtttccggatctgaggggtcgtattggagtcaagagacggagctgcctccacagcgcgaaacgtcaaccccaacaccaacctgaatttgcaatcaccatggggacaagtttcagcagtcaatgggcaattcagacgttgatacggtacccatttgctaagctcaatgacgatccatccaacttcagagaaaggcctttctctggtatgctctggtattcattcgtcttttatcactctcgttgcacaatgcccgggtactcccggaacaagggagtcttccagccaagctgtacagagtgaaaaatagaaatacacctttgcaatcaagacgcgcgttggccaatcacaagacttaatcggtgcaaagaaggattaccaaatttttttttcccaaaatcgctatatagaaataatggaggaaaaagggttaatataaaggagaattcccccgtttttctccccttgtcttttcttcttcaggctttcttacaaatctataatattccaaaatggctgacaacaacaagtctaacttcttcgtcgacttcatg

SEQ ID NO: 126 - DNA, 1003 bp, Pichia pastoris
  misc_feature: GND1 promoter
tatggtagaatcatcaattggaatgaccctatcgttgctatacagactcgtagccccatttcttgtttgttggttttcgtcgtcaattgcctccaaaattgaagagatggcacttcgtcgcgtgggttcctgggaaatattttgaagtggaacatcgttgaaataaggcagtatatcatgctgattctcagcattggagtcagtagcttcaagtgaatctatactgtatgagtcggaagaggatttccggctcgtttttgtcttcattttgttattagaaggaatgataacgttgggaaaccggaggttggagattttgtatatataaaactttcttggagcttattaataaatgcgggatgcagtaaacttgcatatatctattgtaacacttttgcaatagctgcatgccttgactcatcattcagtatcgtgtgaaaaccaatgatacatccgtacattcaaactacaaccttcctcattagtaattctttttgaatttttcggaacccgaagctccgcctatccccccaactaacacatcttccaatttgggtgggagaacacctagcaacatcacgatcattgcgcgaacgttcgcactgtatttttttctcccaaacacccaacttctaggccaaatatccacttctcggggttctattcacccatttaattgttggccttaaaagtcaattgagttccaatcatagtccctagttgattgcttgtagcaaatgccacaacagtaggcatttacgtcctcacagtctcttcccttgtccctcattgatacctctttattctcccccaccaccatacactaccttcctcgcacccctgtcatcacaaccgcaatataatcgatgcgcggtttcttgcctaatccatcgtccaacagagaggtcgctctccttatatatatagttgatccccctttttttctacccttgcaattttttttttgggaccaaagaaaagaaacaagactgatacaaatatg

SEQ ID NO: 127 - DNA, 1003 bp, Pichia pastoris
  misc_feature: GPM1 promoter
gaaagaaggtttatctgactgttgcgcaccaccgaaaccaaaagcgggtttagctgcagaactttcagtggccggcttgttgcccaaagagactgttactgcctccatcggtcttggtagcgcctgaaccaaacgaaaaagcaccactgggattattgtttcctccaaaagacgcggtcgtgtttgatgctgttgaggccgcaggcgcttgaccaaaactaaacgcttttgatgtattgtttgtaggggctgttgcgttgttagaagctggctggttgttcccaaaggaaaaagcacctgttgatccagtgttagatccacctgcggcattcccttgagctgcggagccaaaaattgatccttggtttgagctctgtgtaccaaataaagatgaattggccttagctccgtcagcaggagcgtttgcttgtccaaaggggcctccagacgacgtttgcgtgttgttggcattactagtattggcaggctgatttccaaacaatgagggcttggaggcgttctgggagttaccaaatgagaaccctgtatttccttggccagagcctgagaaactgaaccctgatccagacattatcaataccttgggttattagtagtgtccgttatttttctgtttaggttacgattttgccagattttttgggaggagggaaacaaaagaaccagtgctacacgacctttaagtgccatcaggcatcctgttttctcgacctcatctcatcacatccgtcagtctgagctttcagttctcagttttcgattgactcttgccctgctgcgcgcacaccataccctggctccctctcatgcttctggcgttaccccgggaatcgtacatccatgccgcgaatcccggacaggactcagacggatttcactattcctagtgggcacaacctccatatataaggatacttctgccccctagcttaagtgcctctattttgtcagtaacaactttcaattacacaaacaaacaatg

SEQ ID NO: 128 - DNA, 1003 bp, Pichia pastoris
  misc_feature: HSP90 promoter
ttcaagatcttttgaggactagagagcaagacctgcatttgatagtcgaagaggagaggcaatttaaagatctagtattggagaaggacagactgctcgatgttcaagataagaatgctttgaatctaaggctacaactaggttttgcttcccacaaggtaattgatctaaaccaattacttcctgtgcaaaaaacacttggagccactactagtctggagttaatcaactttcaaacagacatcaaaactttacacgtcgtaaacaatgtcatggacaaaatccgggttaaggtcactggggagaagcgactcgtgacactgactcctgacagcaccaacaaggctctagaatttcccactgagaatgataggccactgcaagaacaacaaaatacagagcatgaaaacgaaaacggtgctcaagatcccatagactcaaaccaaacgaatgattctcagacatactccacacctagccgtatccattatacgactaacgaggagagttcctctaagaaggttaaaatatcaaattgagcatgatacgatagcccgctggcaaggaattaagtgacactagatcgcaaataattcggaatcactttatgtggaatcatcattcttgataaattgtgataaagtatcgttcgcgagatggtttttccagaaatttcttgtggatcaatatctttaaacgatagtaaacgatgttgacagctccaacaatgtcggaactgctcatcaaagacggacaatttccctattagcagaaaaatagcccatttatcatcaccgcagcgctattactgaatgatttttttgaatttttccttgtctgtgaagccgcaaaccacattttctggcgcggttacccggtcatagaagctggtggaatattccatgctacctcgacgatgagcttgcgaaatatatttaagtgtaagaactccgcgtcattaagactctggttaactttttaattttggaaaaatatcaatatg

SEQ ID NO: 129 - DNA, 1003 bp, Pichia pastoris
  misc_feature: KAR2 promoter
ccactatcaaagctatcaattgtggaaatggacagcaaaacggaaacgcagtcaatgacgatgatttcagagctaagcgattgtgcgaaaggtggttggacaatctgtttatgcttctttatgaagatctgagggtgtatacgatgtatcgtgccgaacacatgcacttgacggcacagcaaatggaattcaagaagaccactttagaatgggagttaatagggatggtttcatggaggttaaaacacttcaaggaggcatctgaagcattcaagtatgcactaggtctgaggttttcggtcaaggcatgcaagaaattaattgaattctatctgaacgaacgctccagaatgaaccagccagaaacctcaattgccctcaacaacttaaatcaatccacattatccatccaagagattctcaagtatcgttcgttcctcgatatcaacctaatttcaaacttggtcaaactaggagtttggaatcaccgctggtatgctgagttttctccaaaactcatagaaagccttgcggttgttgtggagaacggagggcttatcaaggtagaaaacgaggttaaggctacctatttcgattcacaagatggagtttacgacttgatgaacgaggtattcaagttcatgaagcattacgattatcctgggactgacaactaagagctcctagtgaagacttgagatggacatgataaacaattatagtgaaaatagaaaccataatacaatattctaatagaggaaccgtttacctgtggttcctattgtggcctactgttactagctagtgtaatacacccttgcctcagctttgcaagttgacaactcagccaaatgatctttgaatgcgcgaaacctcaaggtccatcgaattttctcgaattttcagtgttttcatacagcgtgtcatcttctttcgcgtacttatttaaatcgtacccagatcccttcttcttccttaatttcaattccaacactcaagaatg

SEQ ID NO: 130 - DNA, 1003 bp, Pichia pastoris
  misc_feature: MCM1 promoter
acagctttggcttgaacaatagtggttggatgttacccggtgcgacaagtgctgagttgcggtaatttacgatttcagcgtccaccagaatgggaattccgggtaacacgcataccaggggaagaatatcacaaagatcacagagattggataaaactggaccagaaactaccaaataagtagaccgtcatactcagctcctacaccaagaggtacttcagccttttaccggtttaaaaagccccccgaaatcgactaattaccgaggcattatgtttactactgatggagatttcgaaatatccttcccgattagtccaacatctcgaaattaaactgcgcagcactatgccgaaagctatataaacaatcatttccccaactggaacatcttttttctcttttcttcgtgtatcatcctttggtcttttaatctttcagaaaagttttcattaaaatgcttccagctggtgttattttagtcttttgtctacttttcattatcggggtaattatcgcagttatcctgggcatgaagtggtacaagaagagaaagaactgagcatggacgaagaaaattactagaccaaagatgtatcaccaaacaaacccccccaagaatctgtcataaacgaagttgttataggatggttatcactctcacattttaatacaggaataccctaatttttcctcagttcggtgacgaatggagttgtagttatctttctttccctcgtttctgctcttatttccccctctcgtcgcatcatttgcatcaaattggtgcaacgtgcgcgcacgctcggctttggctgcaacgactttctgtcgtgtaccgacccatattctatcgcttcgggtagtccgcaacaccctcaacttcctaattttgtgttcttacaagctgcaaaaaagtaggactcactcaataaggtaagtccccaaacatcaatcccaattgggtaatccatcaaacactaacccgcattcatttagcatg

SEQ ID NO: 131 - DNA, 1003 bp, Pichia pastoris
  misc_feature: RAD2 promoter
atggtatagtttgcagacatagtaggagagtttgcgggcttgcaactctaacaagtctctgtagtcgctgagactaatgcaatcgtcaagactactgggctcgttatccgaaaaagtcccggagtaactagtcggtaactctctgtaccgaatcatattcatcctggggttgatctgatcttggtccatgtcatcattctctccatttcccgtagggtagggcaatcccaccagccacttgttaaccttttctttatgcggcatattgggagtctgcttggggttgaagctttctggaaactcctttttcttgatcagatcgctgtcactacagaacccaggatgctgctgataaggggaagaaacatagcccatgcccattgagttgaaattgaagggttcctgtgctgcacctgcattcaacggatatacatgtataggatgatggatcatgtggctgtaagaggggccagacgctgagttatcaccattaacattgttaggagatcctgagccttgaaagggcacattgctagtctttcccgtagtcattacaaacagcgggccatagttcccgttgtcacaaggatatgcaattcctgtaggggtatagatgtgtctgttgaatggggtcatggcgggattctagaaaagagacggcccgttagttaaatccacccaccgattattgaggtccaacttacaatttggttttgacggtcttccattggaggatatgttgttctgagttggtggtagttacgtttatttctccttcctttttggtttgtgttagagcaaggacacggagcactgactaccttcaaggccatttgttaagaatccacagatagacactaccacatgggaaatgttgttagggaagagctgatgctgaggctcaacatcgcgcgactccttttagtcagtttccaagtacggttcactgtagacatttctctttttagtttccgtttttccatcaggttgggctaaggatatg

SEQ ID NO: 132 - DNA, 1003 bp, Pichia pastoris
  misc_feature: RPS2 promoter
ctcctgagaacggacagcagcgctggaggcggcctctttaacggtggcggcgaagtcaacaagggtagttttgattttgcctaacaactgttgcaggtccttctcagtctttgggtggctcttcttgaactcgtccgatgcatccttgacgaccttctcagcgtcgtcggcaatctcgtcgacaacttgcactgatccatgagggtcttgggtttgttcgacttctggaactggttttggcttgccagcggaaactccttctgggggtgtggcagcttcggcgtaggtactatcagtgttaatatcggggattggttgatgtgcgagacgaggcaggcgggagatccgtctgcctcgtccaactgcgatagctcttacttacctcatgatgcttgtaaaaaagttaactgaaagatggaaatgggagggggaaaagaattgtggtcaaatccacgtcttgcgataacctcatacatttgctgcatgattgggggatcatcgcaatttcggtccttgtgacacacgcgagctttccgcctggggttagagcgcggagcaagcatactatcaagcaagaaaaaatggtatggagaaacctaattggttaagatatatgaaaactacgacggcttcatacgtggtatctctggttgagctactacgaaccattttcccccttcaaaccctttggcccatgccattcatgccttgcctctctctcaagcaactaagcaatcaagcaatttcccgccttgctgcacatgactgttcggaaatcggagacccaaacaccacttgttatctatgcacgtgatttttatccagggcaataaatactcacttttgcttcaaaacgcttggggcgcgcgagcggcaggctgggaaaaaaataatctcagacttttcaaaagactctcctctttaatcattgaataccgtcgatcgaaaaccaccaccatcggctttccacgtacattccgactttttcagtgtagttaatatg

SEQ ID NO: 133 - DNA, 1003 bp, Pichia pastoris
  misc_feature: RPS31 promoter
ttgtttatagcctataatcgcagactttgttaacccgtaggaataatcctacccactagaattagatcttccaaccatacatagcgtgtactacaacttaagctggtcctcttcttttatcattcgcgcggctctatctcatctacacctccattgaactaatggaaagcaaatacgccgaaaaatatcaagatcgattgattcttggagatgatggcgatgaggacgaattgtttgacgagctggaaaaagatattgaggatcaattcttggccaaatacagagcagagagaatccaacagttaaaacaggagattaccaagatcaaggaccatagttcaaacatcaacctcaatgaccacggtaacatgaaaacaatagatactgatgacgaactattgaaagaaactgttgatagcgaacgtgttgtgatccatttctttaacccatcgtttagcacttgccgtatcatggatgagaagctgtctataatcagcaccaaacatattggaacgcgttttttcagaattgaagcacatagagctccatttttagttgcaaagcttggtatcaaagtgcttccatgtgttgtattgtactacaaaggattagaaagggatagaattgtcggatttgacagattaagtaattctcagaccaattttgagctagaagctttagaggagttactcttagatagtggaattgtggaacgaagaactgtcgattttagcaacctgagaaacaaggtccaaaacaaggtggatcagtcaaaatcagactcagaaagtgatctagatatgtgatagatggcggatggcaggttcattctagtgtttcacgtgacacacgtgagcgtttaagggcacacaccctgactgacgcgcgaacatctaatctgttccgcatgaaaaaaaaaaactacctcgacgaaattctcttctagacagtttttacattggtaagaaagaagcattcacgtattgccgacgaagccaaaatg

SEQ ID NO: 134 - DNA, 1003 bp, Pichia pastoris
  misc_feature: SSA1 promoter
gggttgtatccattcactatttactctttgtttcatttcttgaattatttggatactactctgctggcaactctaccagtctcaaacgcagaccaggttcgcaatttgattagaatgttcgtgagctcttacaatgaaaagtccatgtaccttgcggctagttgtgaattatttttagttccttctttgttgctatcctctttgaagtcgattatattgctggaatggtatagggctcccttttcatttatcaggcaattaatcgtggtattctccgtgatctcgtttctgagattaagatatcaacagaatgtttacatgaaacaattagttgatagttatgatttgaagatcagtcaactcttataccatccccaacttcctcaaggattcaggttgggatatttacgatttaagagtctattaacaagcacgctaggatacttagaattggaaaaaaagaccagataatgagattgaactcgaaatttaggatcacccatatgacgaagaattcatttagattattgaaggtgttttcatgtttacctccatgagaccatttctgtcacagcaaatacaggcaacgcttttcaccagagcttgttggtacaacttttcagatgacgccaaattctcacgcgcctcactttgtgcggcgctaacaataggccatttttttgtacctcccggatggttcagctcaatcactcgattgagaggtttttgttccgcgatttttgttcaccccacacttttctcgaaggttctagcaatcaagataaacaccgcaaagagagccgcaggaaccatatgtggtaccacaagtggtcttaaacaactctggtagaattcgatggaattcgatggaagccgatcgactccgatcgaattgaagcaattcgtatatataaggagaacctagttccaccccttactcgaccattagtttacaagactaacttcacagaagcatagaaattaaacaaagttaaacattatg

SEQ ID NO: 135 - DNA, 1003 bp, Pichia pastoris
  misc_feature: THI3 promoter
atcttttcagcttcatcgtcagtgatatttctcagcccacagaccaagtcaactttggaatctaacaaccttgttcttacaatgttagaactcttaagtcgcatgccatgatcttcaagctgaattttgtgaaggaggtcaaaccccacaatggcatctagttgtttagaatacatgccttcgacaagtgtttgagtgtccaaaatcaagagctcaaaattattgaatttgtctgccaataacgccgtaaattgattagtgtccagcccaccaacaataggagcacctatagttaatttttcagataaatttaagttatcaaggtaaaggagctctaagtttaccccttccaacagggttatttgagaactcaataaattgttgaattcaaaaccaattgtctttgaattctccactggagcttccttgctgaaattgattttgataccattggcatcaaagagacccgtatgataactccataaaaaggggagatgataggccttaaattcatcgttaatctgcaaatttattcctgacatgtctttgtaaatagttatagttcagaaactggaattgagctcaaaaaactggaatcgagcggatatttgaagattgatgccttactcatgaattgattgataagagctccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatcatg

SEQ ID NO: 136 - DNA, 1003 bp, Pichia pastoris
  misc_feature: TPI1 promoter
ttcaacgagacactcttccgtcagttccaaaaccataagtttgccgatgtgttggtccttgtaacgcatggaatttgggccagggtatttttgatgaaatggttcagatggtctgtggaggagtttgaaggcttacgaaatataccacattgccagtttatacagatggttaagggtgaaaatcaacgttacaccttgacgaccccattattacgatggcgtgaaggagatgaagaccgggtagaagaaataagaaaagcggtacagtttaggtccggagatctagggaaggaggccttagcttatattgtagctgctgagagagaggcagctgctggaagatctgaaggccctatcacgtatgatgatggtgatgaccattagagaacgcccagagattgatagccagttcttggacaacaattcggaactttattcacggtgcaaacatgatttgtgtggatagcttcaagtcagacatttcatctcatccccccttttactgctgctaatcaccgttagtccgacagttactctaatcaatatttattagtgttttagttgcgcaaaactcgagcctcttttccttatctcttgacacttcctggagtcgaagtttttcagcgcaaattcactctacaatgtctaccgatactagaccgcctatcttccccctctaaatagcctattggaagggtgcaataaggtatataaatctggcgcgattcccccggacttttatgatccacatcacctcatcttactgccctcactctctttcctgatcctcccaggtccaccgatttcctcactatcgtcggatttctccttccagcgccctagagaattccgtaaccaccgcaaaaatagcagcccccccctcacccatttttttatttaaaagaacaccttactggcccgttttcgtttctcctttactacaattgatttttaattttcagttttttttcattgatatacaagatctatcacaaacacaatg

SEQ ID NO: 137 - DNA, 1003 bp, Pichia pastoris
  misc_feature: UBI4 promoter
agaagattaccataaattgagaactcaaatgaccaagccaggagtcaagaagaattctcaaaagggatacgcaaatattgattcggatgacgaagagctgactgaggctctcgatttcaaattggcaccaaggtcaacaaaaagtctagacataacacggtctggactaccgcaacaatataattttgaaaatgttcttagtagcgacgaagaagatggaggcggagagtttcagggtgattctgtatccccaattaaaaacagttttcattcaagtccaaggaagcagcgatctctactccacgtcatagacgaaggaagtccatctattggcccaatttctgttcaactgaaaagaagtttgacttctcccacaaagagacacaagaatgctcaggaagtgataagccaaccagaaagcatgacatcctcaaacttgttttcgcctacacattcctccgcctcaactacatcaataatatctcccaaaccccaagcggcaatgatcccaagtcttaacccaaataaccctttttatactgatactaatagctaatatgtaataatgattaataaatgcaccactttaatttctttatcaagaatagttttactatttctctttcgggcatatacttgacccgctccatatatttcacctttatcgtgacattatctgctgcacgacccggaattactttaaacttctaagaatttccaaaataggaatcgggatgccgtattttccagtctgctggacagaacttgtgctctctaaaatgaattgtagtggtcctcaaaggctactgctactcacagtctttactactccagattgatccattccttggcccatgcacagtaaattagactactgatctgatgttgtatacttttgagctgattgattacagttaacctggtcttatctaatctctcgtttctttttatctctattcccatttctgtactcttttgttcacgtgacgtttgtcgctttatg

SEQ ID NO: 138 - DNA, 1003 bp, Pichia pastoris
  misc_feature: ENO1 promoter
atgaaagagtgagaggaaagtacctgggcaaaatcacacaattccaaaccatgctaaatgagatttaaagaacaaacgatggcaaaaggcaaccgttataaatgtgatctttcttggcagttatctgtcaatttttctaaggaacagtgaattcatcataggagagatgttatacgttacataatcatacatactgcatgtatctcacctactttacctcatcaactctaaaacagttctagtcccaaccccagattcctagtcatgacacaagtccgcaccggacaggactcacaaccagcaagagaagctaacaaatttacgccccggtaaaacattctttaggggccgttcaatggtaattttcctctcacccgtttaaacttacctccgggcggtatcttcaataacctctgttgtccccgggtatcattggaaacagtgagggacgttgaacagaagagaggatcaccgtaaatttgccttgcaattggccctaaccacggatggttaacttcaagccatcacgacagcaattgagtcggcgcatagctaccctcctcttcttgaccccatgcataggaccaaccttaaccgatggaacaggttcctccgctccgtcccctggtagtgtctctgcgcaagaaatagttaaggtatgaagactgatctctcgcacccccctcacagtactgttatggtgaattgacaaagccattggctagattgaaacatgtaattcatatgtaatcttgttcaattaacgagcttcgtacagtctcaatctagacgtctgataatggcgtttgtgctcctaatcgatgagccatctcatgtgacgtctatacgcttcgatggcttccgtcgcgaatatagaaccacttgaaatatgctgcaaaccacgatccaccctggtcctgaaaagatataaatacagcacatctagcaggcttttgtcttcttggttgaaacacacaattataacaatctacatctaaaaatg

SEQ ID NO: 139 - DNA, 1003 bp, Pichia pastoris
  misc_feature: RPS7A promoter
cctatgtaagccttctcagcatagtcaacaaagaaacctgtttcgacgactcctaccagtcgtatcagcttttcatccagctctttaaccttatttaacggaatctctccaaaatcagcatcaatgacgaagttaccattatctgtgaccactggaccagctttcgcctttccagcatctctcagagtggcgctgttggctcctagtttgaaaagatctttcaatactttaacataactgttgggtacaacctcaatgggaacgcctttgcgccagaaatgacccagtctttctggagactgtttacgaaaatctgccaccacaacgaatttacgagacaatgatgccaccaacttttcttgataaagcgctgcccctcctccctttatcaaattcaagttggtgtcaatttcatcagctccatcaaaggctacgtctatctcatcgaactcctccacatttcctaaacgaaggccattgtccaagataagttgctttgattgaaagctggtagggatacaaataaacttttctttattttccaactgccccaagcgttcggcgacatagacaacggtggatccagacccaactcctataattctgtcagacttcgaaacgttttcattgactgctgcgaaagcagccaatttttttgacttctcaattagttcctcagacattagttgtctaagatgctagaggcaagacgtgaataagtacccacaaaggacggtctaatttcccttacattcatcaataggctatttcaagcatgaacttacataagcgtattgtgcattagatattaacctcttagcatcccaacaccgtactgggcttcgaccctaaccacatcccctacatcacatgactgtaattttttttgttactactcttctcttgggactgttgtgcgcgaatccagtttttcctccagagccttaacaatcttttttcaattctgtcgaaactcacacggtaacagcagtatcacaagatg

SEQ ID NO: 140 - DNA, 1003 bp, Pichia pastoris
  misc_feature: RPL1 promoter
cataagtcctagaacaccacttgttagtaaaaccggtaaaattccccattttctcatattcaacttacaatgctcaaaagatgatctttgaaggccatcccagtggtgttagccatcctatggaagggtttgttagtaattgctgatccaaaacctgaagactgaaaaaaaggctcgcacatgtcactcattaccacatccacagggaattgctgcttctgcttgttacttatctcgaggttatcgtctacttcagcctgaaagtaggaaggtccatccgctattggttcaatttcttgaagctttctaaggtctttgtccatgttcaactggtaaaaatgagtctttatcatctcatgcgtggtcttactcaagatgttggcctgcattgaagacaccccttttggaggctcacaggctaggatatctatcccaagaatattgcctctggaagtcactttatcggctgcaacttgggaccatgcaccaggagcaaaccccaaatccacgacattttgccctggacggaagatattgaacttctcatggatttggagcagcttaaacgctgcccttgatttcaatgcctttgcgtgagcttcctttgtgaaggggtcagatgattgacgttccagccatttcctagaggatgaactctttccttttactttcagggaaacaaacctagcaaactgcctccgtagcagcgacacagaaggatgctgtatcatgtgatgaacgtagctaagcacaacctgtgtgacaattccgtgtcgtttatcttatcttgctagtctagatataaagtatcacgtgacaaatattatttttgccccaacaccggcttgaaacttagggctcgcgccctcgtcgcgagaattcttacctttcgttaaattttttttcagtgttccaacttttcccttctctatccatcgtcgaatcaaacctttacgggattacctacaacctttttgctagggagacgaattaatagaaaatg

SEQ ID NO: 141 - DNA, 1009 bp, Pichia pastoris
  misc_feature: TKL1 promoter
gatatcgattccactgctcagagtcttttcatcaagctctaagaaaagtccggggatggaggagccagcgcaacagaaaactatgtcgggactaaaaccaacttcctctaaaacacgctcacactggtcatatttggagatatctgctgccacgtaagaaattgtgttgcccttctcggtcccgtgaatatcaatagcgtcctttactacctgctttagtaaggattctgttctagccacaatcacaacggaacatccttttccatacaaaagtttagcaaactcggctccaacaccctgggatgcacccgtaataattgcctttttgttggtgacatcaaaattatcaaacattccagttccctctacactttgtatgagaatgatagctgaaattgtgcaccagatgttagaagataaggtcgtgtcatgaactaatatcatgaattccgagggtggctcaacaactattcacgtgacttggacgttggaagttgaggtggttggtggatgttgcacggagtatcatttgtaagcatgaaatcagtctaaaaaacttgcagaatagcagagcggttcggaaattcattcaaaaccacctcctcagattggatctgccctactctgtttagctctgggagattttctcggtcgtgttctttcgctggtctacccacgctataggaatcgctgtgaacgctaccttcttcccaacttctcggtgactattataagccattcccactttgttttcaagcaccaacaacccacccccaccttatctactccatcttgggtgtccccgcgcctgttgcaaagtccgaaccatagaacccccgacctttgtcccactaaccctcagacacccctcggaagtcagggagaaaccactccgaagtacattaatcatccctcgtattctcgacggtgcccattttctttataaaaagggagacacaggttgcttcactaactctagacttgtattctacatccactctacacaatg

SEQ ID NO: 142 - DNA, 1003 bp, Pichia pastoris
  misc_feature: PIS1 promoter
tttttcctcttcgttgtgtggtaaactcggtaacgaggctaaaagtttttgcaaatttgagtcttcattcaaatctaagaactctgaataaatctcaaacacttctttcatttcgatcaataagtgtagtagtttctgtctataaatttcttcggtgtttgttctttctagagcgtattgtatactaacaaagttggatacgaccaaagtttggagctcattgtccattttgtgtaatccttcatcgtcatcaagcagaattcttagctccttttggatcttggttgtaaaaggtctcaacgttgtagtattgttcataagaagcttctgtaacacaaaattagctggcttggggcaaagatacatcacgctaacaagcgattgaataattgacggaagccttggtgtcagtatttctctggtaagtgcaaccttatgccttattttgtccataataaaatctagagtggatatagctgattccaaacaggtcatctgtcgtacatcaatggttgatgtgatgtaacacttcgactcaataatcttggtcaaagcactaatatagttaccagcgtccgatgcaataatccttgggtgaaggcaaaggacgtggacaagttttgtgccaaaccaacggacttcagattgatttgatctaagataatttgcagcacgagaggtaacatgcgtcaagtcggcttttgaagcttcattaacggcgttctcatcagcaaggatggggagtatttccatcagatccgcctcaactgaattatccttcaaatgcggaatgacaacgtcgagtccaacaaatccttgatatcccatcctggaagtccaattgtatgatctaaagagattacttcattctattttttttttttttttattggaatttttcagcccaaggctctctctatcagtgtaccgaccaatacgtatcagatacagataattttgagctcaggcaacatggaaatttacttgtctctagatagtcatg

SEQ ID NO: 143 - DNA, 1003 bp, Pichia pastoris
  misc_feature: FET3 promoter
tgggctttcgtaccaaatggaaaaatcacgtacaagtatgcccagagctaagctaatcggatggcaagtagaaagtggatgggtttcacagaacaataaagaatgagtgacggatattattggctggcaggcattaaagatgcataatttaagctttctgttttctacttttggaattgtcacaaatttgaactgtggatgttattgaaacacagacccgtataaatacctcttgagagaaatttgaaagtgaagctatttcagtgaattaatcactcgccatacacgaggtagataattcacgtagacgaatttcttttgatccattttattcagggtggacagtcagaagtgttcgttcacctgatatgttctagatgcagctcgaaacgctgtaaaaaaaaaaagtcccaaaagtcacgtgcataaaggtgtagttcaatttaatggagataacataacatctatgactcctttcatgaatccatctcaaaaacacaaactttgctagaatatctggtggcaccgatttttcatcatttcacgagtttatatagcatatgcgccaacagaacgttgcctgacacaatgttaaggcttttaaattttgcttgtgtagtaaaaagttagtagtgtgttactcgatatcatatttctatcagaagtggaatattctaatctctcctctacctttgttacaatccgtttcgaacagaaaaaagaatttatgatgattttatggagaatcattccaataatagcattgctaattagattgacggtagcaaagactcacaagttcaatttgacagcttcttgggtgaaagcaaatccagatggtgtttttgagagagatgtcattggacttaacggacagtggcctctaccagttctaagggtgaatcaaggagacaggatcgaactgttgttgacgaatggtcttggcaatgcgaatacatctttgcatttccacggcctgttccaaagaggatctagttttatg

SEQ ID NO: 144 - DNA, 1003 bp, Pichia pastoris
  misc_feature: FTR1 promoter
tcgagtaacacactactaactttttactacacaagcaaaatttaaaagccttaacattgtgtcaggcaacgttctgttggcgcatatgctatataaactcgtgaaatgatgaaaaatcggtgccaccagatattctagcaaagtttgtgtttttgagatggattcatgaaaggagtcatagatgttatgttatctccattaaattgaactacacctttatgcacgtgacttttgggactttttttttttacagcgtttcgagctgcatctagaacatatcaggtgaacgaacacttctgactgtccaccctgaataaaatggatcaaaagaaattcgtctacgtgaattatctacctcgtgtatggcgagtgattaattcactgaaatagcttcactttcaaatttctctcaagaggtatttatacgggtctgtgtttcaataacatccacagttcaaatttgtgacaattccaaaagtagaaaacagaaagcttaaattatgcatctttaatgcctgccagccaataatatccgtcactcattctttattgttctgtgaaacccatccactttctacttgccatccgattagcttagctctgggcatacttgtacgtgatttttccatttggtacgaaagcccacgaaccacacgactgtacttgacttttggggtcgttaatgtgcacagcccagagatgatctgataattaatatcatttcgcaccactgtttaaaaaattcgataatttgttactaaaatgctatttttgatgcaatgcgtgcatgttcatgcaccagtagataataatcttaaatttacaatatagaagctagtttcttaaagttttattggcttatgtgttttagggagaaaagtttcagagctatataaactcgatccttccttccacaaatctcgccttcaagtcatttctcgaacttccttccaatagcagtgccactcaacgttcagtggtcgttgtagttcttttcaaaatg

SEQ ID NO: 145 - DNA, 1003 bp, Pichia pastoris
  misc_feature: NMT1 promoter
tcgacacacagaggaaacgagggtaacatgatatcatgatgtacgtacaaactaggatctctggtcattccacgggccacatcaggatcgaatttgtcgccgtcctcttcttcagcgccatcaggggctccagcaccgtcgtcgtatacgtaaacatctaaaaaggagatatcgtcttcagttctggtggaaaggatcatattgtctgtggggtatacttgcaactcttgtttctcttctaaaatttcacgttccgttggaagagtgatgtatgcgtcaccatctgcgttgttctcatcttcgtggtaacgggcacccgtacttgataacccaggtagcatcgagactttgaggccattggcaacagaatcctcatacgtttcgtcatcatcgtaatgttctaaatcatattcctttagatcatcatcaatgtcatcttgagactgtagtttgctagctatagatttgttgatgttggctttcatttctgaaatatcagaatcatctttctgtgtttcggccagctcatcctgggcatctttcagtttcaaattggccatggcttcaattctctccatctcaacatcgttcatttcatatttctcgggaaactccgaggggaagcctctgggaacccatgttgtagacgaaatcataagtgtacaatactgtctctttaaactgaaaaaaaaaatttgagaagaaaaaaaaaattgctggtcacatgataaggtggatcccgtaatcaagcattaacctggttcaggttcgtggattgtttaggctttataaaattgactggtaggtccccagtttcaagttttctttagtaatctactcgcacaaattattgttgggcatctcgtcctatcagattcatatagatctataattgattcccctcctgaaacactttcaaccaacatctgagggttctcgaaaattcagtaagattgactcccttctcatccaggcttggtgaccatcaaaatcaccaatcatg

SEQ ID NO: 146 - DNA, 1003 bp, Pichia pastoris
  misc_feature: PHO8 promoter
ggtataagtatagcacatgttgacgctgatataatcatcccaaaaaagtgtgcggtttatttagaaaggagaagaggaactcatgaaatttttgaaggaacgcacccattctaggtgatgtacgggtgtcttttgataagtagtggataggttttattcactggactatatggtactgtacaaaagaagttcctcttcagatggtctgtagcttgcatgcaatgttctctctaaggaatgtgtatttgaaaggtcatgacttaccataaccagtcgcaaaatgcttttgttccaggttctcttcctgaggaacgttttgacggggtatacataccttatcgagatcaattttaggaatagatactttctctccaaccgttaaaaatttggaaatcttcactttggcaggttttaactcgtcaagagacttgacgtctaggatcttatacttgcttatgtcgttcttggtgtcctgatcgagagagtcttccctgacttggtatttcttaccttcgtgcttaaaactatgagccgttttgtccttaatgggcatgggaagagttttcaatgtgcttaaatccaaatctttgggagttttcaacagccatacttctttctttcccaaatccttgaacggagatatttctttgaggtctgtgcgctgtttgaaattagcaggtggttcaaaagtgaaatcctctgaatcactgctttcagagttgtcggagctaatatcggactctgatataaactcccttggatattcttttcatttcagagacaccctaatagttgtaggaaagattaaaccaagtgaaaaaaaaaattcaatcgcgatggtagagtctggaaggagatgttctccttctggctcacatctcgctaagtggacattcttcacggaactacgcacaaatctatgcccctttcaggatctcgcgcaatctgcattgatatgtgatatttgttttagcgtcctctccccttcaatttcaaagcaatg

SEQ ID NO: 147 - DNA, 1006 bp, Pichia pastoris
  misc_feature: FET3pre promoter
ttgtggtcctatgaattaaccatttaaaaatattgctattaatgattatgttaggtggattaatgttgtcattagtacatatattcgtattttaacttaattactcattaattaagtgtagaatgtcatttcactggatttaacttgctttgtaagtacgaaacaatgctcgatcaataatcaaacagtttagttcagagattagattcaaatataattctaaatttatagtatcagaaatatatattaaggacagtgttaattttacctcatttaaactgctgctagccattaaacgattgtgcttttttagtccatgtttaagaacaaatgaccatttgctaaacctactgtaatcttattataaaaatgaagcctacaaatcatttcttacaagcaaatgattttacagccccttgtgctgaatgtatagtacacattggacgaggagaaacgatctaagtggccactcgtaggtgttaatgatactattcgctcgaacttaacggtgcttaggattctacttaatccttgcacgtgacaaggcttaaattaagcttgttttctgatgtgtgtcaaaaaaattttagcttgacatatatcgaacgatacggcgtattcttaggcaacaatttcaacttcttcgcttatttgcaatactggtatccacggggtaatgccaaacttaacaaacagtaatggatttgaagctagcatgctgtatttcaaggagcggagaagcaaagatagccccccttagattaaatgcatgtttaagctttcgaggcacttttctgattaatgcataagcctgcaggaaaggattccacttatttagtaaaattatcagttcaatcgccaaataaaggcacaacaagaaaaagaaacaaaacaataaaatttcaaccttctttcagcgtatataaaagaaatttactgcccacttcctcgaagttttcctttcccctagctgtaaccactgccgccaacactccatcgaagaatg

(19) United States
(12) Patent Application Publication
(10) Pub. No.: US 2010/0297738 A1
(43) Pub. Date: Nov. 25, 2010

(54) EXPRESSION SYSTEM

(75) Inventors: Brigitte Gasser, Wien (AT); Diethard Mattanovich, Wien (AT); Michael Sauer, Wien (AT); Gerhard Stadlmayr, Wien (AT)

Correspondence Address: OLIFF & BERRIDGE, PLC, P.O. BOX 320850, ALEXANDRIA, VA, 22320-4850, US

(73) Assignee: POLYMUN SCIENTIFIC IMMUNBIOLOGISCHE FORSCHUNG GMBH, VIENNA (AT)

(21) Appl. No.: 12/450,705

(22) PCT Filed: Apr. 17, 2008
(86) PCT No.: PCT/EP2008/003076

§ 371 Date: Oct. 8, 2009

(30) Foreign Application Priority Data
Apr. 20, 2007 (EP) 07008051.0

(51) Int. Cl. C12N 1/19; C07H 21/04; C12N 15/81

(52) U.S. Cl. 435/254.2; 536/24.1; 435/320.1

(57) ABSTRACT

The present invention relates to methods for increasing the secretion of a protein of interest (POI) from a eukaryotic cell comprising co-expression of a POI and of at least one protein that enhances protein secretion, said enhancing protein being selected from the group consisting of BMH2, BFR2, C0G6, C0Y1, CUP5, IMH 1, KIN2, SEC31, SSA4 and SSE1. The invention further relates to a yeast promoter sequence, in particular to a promoter sequence of the PET9 gene of P. pastoris, having, under comparable conditions, an increased promoter activity relative to a promoter sequence of the GAP protein. The invention further relates to an expression vector comprising such a promoter sequence and to the use of such an expression vector for expression of a POI in a host cell. The invention further relates to new yeast promoter sequences of genes from P. pastoris, which are useful for expression of a POI in yeast.

### TECHNICAL FIELD

The present invention is in the field of biotechnology, in particular in the field of gene expression and relates to a method for increasing the secretion of a protein of interest (POI) from a eukaryotic cell, comprising co-expression of a recombinant nucleotide sequence encoding a protein of interest and at least one recombinant nucleotide sequence encoding a protein that increases protein secretion. The invention further relates to a yeast promoter sequence, in particular to a promoter sequence of the PET9 gene of Pichia pastoris (P. pastoris), which is particularly useful for expression of a protein of interest in yeast, preferably in a strain of the genus Komagataella (Komagataella pastoris, Komagataella pseudopastoris or Komagataella phaffii), and which has an increased promoter activity relative to the promoter sequence of the glycerol aldehyde phosphate dehydrogenase (GAP) gene of Pichia pastoris under comparable conditions. The invention further relates to an expression vector based on the pPuzzle backbone comprising a PET9 promoter sequence from P. pastoris, as well as to the use of such an expression vector for expression of a protein of interest in a host cell, in particular in a strain of the genus Komagataella (K. pastoris, K. pseudopastoris or K. phaffii).

The invention also relates to new yeast promoter sequences of genes from P. pastoris, which are useful for expression of a protein of interest in yeast, preferably in a strain of the genus Komagataella (K. pastoris, K. pseudopastoris or K. phaffii).

### BACKGROUND OF THE INVENTION

Successful secretion of proteins has been accomplished both with prokaryotic and eukaryotic hosts. The most prominent examples are bacteria like Escherichia coli, yeasts like Saccharomyces cerevisiae, Pichia pastoris or Hansenula polymorpha, filamentous fungi like Aspergillus awamori or Trichoderma reesei, or mammalian cells like e.g. CHO cells. While the secretion of some proteins is readily achieved at high rates, many other proteins are only secreted at comparatively low levels (Punt et al., 2002; Macauley-Patrick et al., 2005; Porro et al., 2005).

The heterologous expression of a gene in a host organism requires a vector allowing stable transformation of the host organism. This vector has to provide the gene with a functional promoter adjacent to the 5′ end of the coding sequence. The transcription is thereby regulated and initiated by this promoter sequence. Most promoters used up to date have been derived from genes that code for metabolic enzymes that are usually present at high concentrations in the cell.

EP 0103409 discloses the use of yeast promoters associated with expression of specific enzymes in the glycolytic pathway, i.e. promoters involved in expression of pyruvate kinase, triosephosphate isomerase, phosphoglucose isomerase, phosphoglycerate mutase, hexokinase 1 and 2, glucokinase, phosphofructose kinase, aldolase and glycolytic regulation gene.

WO 97/44470 describes yeast promoters from Yarrowia lipolytica for the translation elongation factor 1 (TEF1) protein and for the ribosomal protein S7 that are suitable for heterologous expression of proteins in yeast.

WO 2005/003310 provides methods for the expression of a coding sequence of interest in yeast using a promoter of the glyceraldehyde-3-phosphate dehydrogenase or phosphoglycerate mutase from oleaginous yeast Yarrowia lipolytica.

One approach for the improvement of the secretion of a recombinant protein was done by random mutagenesis (Archer et al., 1994; Lang and Looman, 1995). The major disadvantage of this method is that positive results usually cannot be transferred to other strains.

The secretory pathway—the folding and processing of proteins—of eukaryotic organisms, e.g. of yeast, is very complex with many interacting participants. Some of these proteins have catalytic activity on the proteins like protein disulfide isomerase (PDI), others act by binding to the proteins and preventing them from aggregation (chaperones, e.g. BiP), or by stimulating release of the protein to the cell exterior at a later step in the secretory pathway (SSO proteins). Due to this interdependence, increasing the rate of one reaction step in the secretory pathway may not automatically augment secretion of a protein of interest, but instead may cause a rate-limitation at one or more of the subsequent reaction steps and thus may not remove but only shift bottle-neck(s) of the expression system.

The secretory pathway typically starts by translocation of transmembrane polypeptides and polypeptides intended for secretion into the lumen of the endoplasmatic reticulum (ER). For that purpose, these proteins possess an amino-terminal signal sequence. This signal sequence—also called leader sequence—typically consists of 13 to 36 rather hydrophobic amino acids; no special consensus sequence has been identified yet. On the ER luminal side the signal sequence is removed by a signal peptidase, while the nascent polypeptide is bound to chaperones to prevent miscoiling until translation has finished. ER resident proteins are responsible for correct folding mechanisms. They include, for example, calnexin, calreticulin, Erp72, GRP94, and PDI which latter catalyses the formation of disulfide bonds, and the prolyl-isomerase. Besides, some of the post-translational modifications such as N-glycosylation are initiated in the ER lumen. Proteins are exported to the Golgi apparatus by vesicular transport only after the correct conformation of the proteins has been assured by the ER quality control mechanism. Unless there is a differing signal, proteins intended for secretion are directed from the Golgi apparatus to the outside of the plasma membrane by specific transport vesicles (Stryer and Lubert, 1995; Gething and Sambrook, 1992).

In most cases the rate limiting step in the eukaryotic secretion pathway has been identified to be the move of proteins from the ER to the Golgi apparatus (Shuster, 1991). A mechanism called ER-associated protein degradation (ERAD) is responsible for the retention of misfolded or unmodified non-functional proteins in the ER and their subsequent removal.

It has been shown in several cases that the secretion process of heterologous proteins can be enhanced by co-overexpression of certain proteins that are involved in the secretory pathway and which support the folding and/or processing of other proteins (Mattanovich et al., 2004).

Co-expression of the gene encoding PDI and a gene encoding a heterologous disulphide-bonded protein was first suggested in WO 93/25676 as a means of increasing the production of the heterologous protein. WO 93/25676 reports that the recombinant expression of antistasin and tick anticoagulant protein can be increased by co-expression with PDI.

WO 94/08012 provides methods for increasing protein secretion in yeast by increasing expression of a Hsp70 chaperone protein, i.e. KAR2 and BiP or a PDI chaperone protein.

The yeast syntaxin homologs SSO1 and SSO2 are necessary for the fusion of secretory vesicles to the plasma membrane by acting as t-SNAREs.

WO 94/08024 discloses a process for producing increased amounts of secreted foreign or endogenous proteins by co-expression of the genes SSO1 and SOS2.

WO 03/057897 provides methods for the recombinant expression of a protein of interest by co-expressing at least two genes encoding proteins selected from the group consisting of the chaperone proteins GroEL, GRoES, Dnak, DnaJ, GRpe, CIpB and homologs thereof.

WO 2005/0617818 and WO 2006/067511 provide methods for producing a desired heterologous protein in yeast by using a 2 μm-based expression plasmid. It was demonstrated that the production of a heterologous protein is substantially increased when the genes for one or more chaperone protein(s) and a heterologous protein are co-expressed on the same plasmid.

Another approach to stimulate the secretory pathway is to overexpress the unfolded protein response (UPR) activating transcription factor HAC1. Transcriptional analyses revealed that up to 330 genes are regulated by HAC1, most of them belonging to the functional groups of secretion or the biogenesis of secretory organelles (e.g. ER-resident chaperones, foldases, components of the Translocon).

WO 01/72783 describes methods for increasing the amount of a heterologous protein secreted from a eukaryotic cell by inducing an elevated unfolded protein response (UPR), wherein the UPR is modulated by co-expression of a protein selected from the group consisting of HAC1, PTC2 and IRE1.

The flavoenzyme ERO1 is required for oxidation of protein dithiols in the ER. It is oxidized by molecular oxygen and acts as a specific oxidant of PDI. Disulfides generated de novo within ERO1 are transferred to PDI and then to substrate proteins by dithiol-disulfide exchange reactions.

WO 99/07727 discloses the use of ERO1 to enhance disulfide bond formation and thereby to increase the yield of properly folded recombinant proteins.

While these approaches, once established, can be transferred to other strains and used for other proteins as well, they are limited by the actual knowledge about the function of such proteins supporting the secretion of other proteins.

It can be anticipated that the successful high level secretion of a recombinant protein may be limited at a number of different steps, like folding, disulfide bridge formation, glycosylation, transport within the cell, or release from the cell. As many of these processes are still not fully understood, it can also be anticipated that there are many more proteins involved which support the secretion of a protein, than is currently known. However, such helper functions cannot be predicted with the current knowledge of the state-of-the-art, even when the DNA sequence of the entire genome of a host organism is available.

Proteins known to be involved in the yeast secretory pathway frequently influence the process of protein folding and subsequent secretion at different steps of the secretion process.

Accordingly, it is desirable to provide new methods to increase production of secreted proteins in eukaryotic cells which are simple and efficient. It is also desirable to provide new genes to be used in methods for the increased production of secreted proteins. It is also desirable to provide new yeast promoters, especially for use in the expression of heterologous or homologous genes in yeast, in particular in a yeast of the genus Komagataella, but also for expression of a desired gene in any other eukaryotic expression system.

### SUMMARY OF THE INVENTION

It is an objective of the present invention to provide a method of increasing the secretion of a protein of interest (POI) from a eukaryotic cell, comprising co-expression of a recombinant nucleotide sequence encoding a POI and at least one recombinant nucleotide sequence encoding a protein that increases protein secretion from a host cell. An increase in secretion of the POI is determined on the basis of a comparison of its secretion yield in the presence or absence of co-expression of a said protein that increases protein secretion.

In one aspect the invention relates to such a method including the co-expression of a recombinant nucleotide sequence encoding a POI and of at least one other recombinant nucleotide sequence encoding a protein that increases protein secretion, wherein said protein that increases protein secretion is selected from the group consisting of BMH2, BFR2, C0G6, C0Y1, CUP5, IMH1, KIN2, SEC31, SSA4, SSE1, and a biologically active fragment of any of the foregoing proteins.

In another aspect the invention relates to such a method wherein at least one other recombinant nucleotide sequence is obtained from a yeast, preferably from Saccharomyces cerevisiae or from Pichia pastoris.

In another aspect the invention relates to such a method wherein at least one recombinant nucleotide sequence encoding a protein that increases protein secretion is obtained from Saccharomyces cerevisiae and is identical with or corresponds to and has the functional characteristics of a sequence selected from the group consisting of SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40 and SEQ ID NO 41.

In another aspect the invention relates to such a method wherein at least one recombinant nucleotide sequence encoding a protein that increases protein secretion is obtained from Pichia pastoris and is identical with or corresponds to and has the functional characteristics of a sequence selected from the group consisting of SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50 and SEQ ID NO 51.

In yet another aspect the invention relates to the use of such a nucleotide sequence encoding a protein that increases protein secretion as a protein secretion enhancer, particularly as an enhancer of the secretion of a POI from a eukaryotic cell.

It is another object of the invention to provide a nucleotide sequence encoding a protein that increases protein secretion from a host cell, wherein the nucleotide sequence is isolated from Pichia pastoris and is identical with or corresponds to and has the functional characteristics of a sequence selected from the group consisting of a nucleotide sequence encoding the protein BMH2 (SEQ ID NO 42), a nucleotide sequence encoding the protein BFR2 (SEQ ID NO 43), a nucleotide sequence encoding the protein C0G6 (SEQ ID NO 44), a nucleotide sequence encoding the protein C0Y1 (SEQ ID NO 45), a nucleotide sequence encoding the protein CUP5 (SEQ ID NO 46), a nucleotide sequence encoding the protein IMH1 (SEQ ID NO 47), a nucleotide sequence encoding the protein KIN2 (SEQ ID NO 48), a nucleotide sequence encoding the protein SEC31 (SEQ ID NO 49), a nucleotide sequence encoding the protein SSA4 (SEQ ID NO 50) and a nucleotide sequence encoding the protein SSE1 (SEQ ID NO 51).

It is another object of the invention to provide a yeast promoter sequence of the PET9 gene from Pichia pastoris, which is useful for expression of a POI in yeast, preferably in a strain of the genus Komagataella, in particular in a strain of K. pastoris, K. pseudopastoris or K. phaffii, and which has, under comparable conditions, an increased promoter activity relative to the promoter sequence of the GAP protein of Pichia pastoris.

It is another object of the invention to provide such a yeast promoter sequence, particularly a yeast promoter sequence identical with or corresponding to and having the functional characteristics of SEQ ID NO 125, or a functionally equivalent variant thereof.

In another aspect the invention relates to an expression vector based on the pPuzzle backbone further comprising such a yeast promoter sequence of the PET9 gene from Pichia pastoris which is identical with or corresponding to and having the functional characteristics of SEQ ID NO 125, or a functionally equivalent variant thereof.

In yet another aspect the invention relates to the use of such a plasmid for the expression of a POI in a host cell, the host cell preferably being a cell of a strain of the genus Komagataella, in particular a cell of a strain of K. pastoris, K. pseudopastoris or K. phaffii.

It is another object of the invention to provide a yeast promoter sequence from Pichia pastoris which is useful for the expression of a POI in yeast, preferably in a strain of the genus Komagataella, wherein the yeast promoter sequence is identical with or corresponds to and has the functional characteristics of a sequence selected from the group consisting of a 1000 bp fragment from the 5′-non coding region of the GND1 gene (SEQ ID NO 126), a 1000 bp fragment from the 5′-non coding region of the GPM1 gene (SEQ ID NO 127), a 1000 bp fragment from the 5′-non coding region of the HSP90 gene (SEQ ID NO 128), a 1000 bp fragment from the 5′-non coding region of the KAR2 gene (SEQ ID NO 129), a 1000 bp fragment from the 5′-non coding region of the MCM1 gene (SEQ ID NO 130), a 1000 bp fragment from the 5′-non coding region of the RAD2 gene (SEQ ID NO 131), a 1000 bp fragment from the 5′-non coding region of the RPS2 gene (SEQ ID NO 132), a 1000 bp fragment from the 5′-non coding region of the RPS31 gene (SEQ ID NO 133), a 1000 bp fragment from the 5′-non coding region of the SSA1 gene (SEQ ID NO 134), a 1000 bp fragment from the 5′-non coding region of the THI3 gene (SEQ ID NO 135), a 1000 bp fragment from the 5′-non coding region of the TPI1 gene (SEQ ID NO 136), a 1000 bp fragment from the 5′-non coding region of the UBI4 gene (SEQ ID NO 137), a 1000 bp fragment from the 5′-non coding region of the ENO1 gene (SEQ ID NO 138), a 1000 bp fragment from the 5′-non coding region of the RPS7A gene (SEQ ID NO 139), a 1000 bp fragment from the 5′-non coding region of the RPL1 gene (SEQ ID NO 140), a 1000 bp fragment from the 5′-non coding region of the TKL1 gene (SEQ ID NO 141), a 1000 bp fragment from the 5′-non coding region of the PIS1 gene (SEQ ID NO 142), a 1000 bp fragment from the 5′-non coding region of the FET3 gene (SEQ ID NO 143), a 1000 bp fragment from the 5′-non coding region of the FTR1 gene (SEQ ID NO 144), a 1000 bp fragment from the 5′-non coding region of the NMT1 gene (SEQ ID NO 145), a 1000 bp fragment from the 5′-non coding region of the PHO8 gene (SEQ ID NO 146), and a 1000 bp fragment from the 5′-non coding region of the FET3 precursor (FET3pre) gene (SEQ ID NO 147), or a functionally equivalent variant of any of the foregoing sequences.

In another aspect the invention relates to an expression vector based on the pPuzzle backbone further comprising such a yeast promoter sequence identical with or corresponding to and having the functional characteristics of a sequence selected from the group consisting of SEQ ID NO 126, SEQ ID NO 127, SEQ ID NO 128, SEQ ID NO 129, SEQ ID NO 130, SEQ ID NO 131, SEQ ID NO 132, SEQ ID NO 133, SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 143, SEQ ID NO 144, SEQ ID NO 145, SEQ ID NO 146 and SEQ ID NO 147, or a functionally equivalent variant of any of the foregoing sequences.

In another aspect the invention relates to the use of such an expression vector for the expression of a POI in a host cell, the host cell being a cell of a strain of the genus Komagataella, in particular a cell of a strain of K. pastoris, K. pseudopastoris or K. phaffii.

The principle of the invention is further described in the independent claims, while the various embodiments of the invention are the subject matter of dependent claims.

### BRIEF DESCRIPTION OF THE DRAWINGS

FIG. 1 shows the structure and relevant restriction enzyme cleavage sites of the vector backbone of pPuzzle, comprising a AmpR selection marker for E. coli amplified from the cloning vector pBR322 and an E. coli origin of replication (ORI) amplified from the cloning vector pUC19. A detailed description of the cloning procedure of the pPuzzle vector backbone is found in Example 3.

FIG. 2 shows the structure and relevant restriction enzyme cleavage sites of the vector pPuzzle_zeoR_P_{PET9—}eGFP_AOXTT, where the reporter gene GFP (green fluorescent protein) is under the control of a 1000 bp fragment from the 5′-non coding region of the PET9 gene of P. pastoris. The vector further comprises an E. coli ORI amplified from pUC19, the transcription terminator of the cytochrome c gene from S. cerevisiae (cyc1TT), a zeocin selection marker and the promoter sequence of the AOX1 gene of P. pastoris (AOXTT_part 1 and 2).

### DETAILED DESCRIPTION OF THE INVENTION

To understand more about the gene regulation of a host organism during protein production, DNA microarray hybridization experiments with P. pastoris clones expressing recombinant human (rh) trypsinogen in comparison to a non-producing strain (according to Sauer et al., 2004) were performed. A detailed description of the experimental procedure is found in Example 1. These experiments allow for a determination of the transcription levels of approximately ⅓ of all genes in P. pastoris, but they do not provide direct information on the potential of any hitherto unidentified protein to enhance secretion.

Additional analysis of the data derived from DNA microarray hybridization has allowed the identification of potential secretion supporting proteins, or their genes respectively. To achieve this, the relative expression levels of all measured genes of a P. pastoris strain being transformed with a plasmid carrying a gene for rh trypsinogen were compared to a wild type strain cultivated under the same conditions. Then the genes were ordered by the relative difference of their expression levels, and some 524 genes with the highest difference were considered for further analysis. As the DNA microarrays used for these experiments were derived from Saccharomyces cerevisiae gene sequences, only putative gene functions for P. pastoris can be assigned by the homology to S. cerevisiae. After ranking the 524 differentially regulated genes based on their putative intracellular localisation and function, and focusing on those being involved in secretion and/or general stress response, out of a number of 64 potentially interesting genes 15 were selected for further analysis. These genes were cloned from S. cerevisiae by PCR and subcloned into a P. pastoris expression vector, and subsequently transformed into a P. pastoris strain expressing the Fab fragment of a monoclonal antibody (2F5mAb) against HIV1. By cultivating the clones producing both the Fab fragment and the different putative secretion helper proteins, compared to clones producing only the Fab fragment, a beneficial effect of the overexpression of the following genes encoding putative helper proteins on the secretion of the Fab fragment could be identified: PDI1, CUP5, SSA4, BMH2, KIN2, KAR2, HAC1, ERO1, SSE1, BFR2, C0G6, SSO2, C0Y1, IMH1 and SEC31.

The proteins PDI1, KAR2, HAC1, ERO1 and SSO2 are already known in the art as being successfully applicable folding/secretion helper factors when co-expressed during recombinant expression of heterologous proteins.

The other proteins identified in the DNA microarray assay, i.e. CUP5, SSA4, BMH2, KIN2, SSE1, BFR2, C0G6, C0Y1, IMH1 and SEC31 have not yet been described as having a beneficial effect on the secretion of recombinantly produced POI.

Accordingly, the present invention in its first aspect relates to a method of increasing the secretion of a POI from a eukaryotic cell comprising:
providing a host cell comprising a recombinant nucleotide sequence encoding a POI and at least one recombinant nucleotide sequence encoding a protein that increases protein secretion; and
expressing in the host cell the recombinant nucleotide sequence encoding a POI and the at least one recombinant nucleotide sequence encoding a protein that increases protein secretion,

wherein said protein that increases protein secretion is selected from the group consisting of BMH2, BFR2, C0G6, C0Y1, CUP5, IMH1, KIN2, SEC31, SSA4, SSE1, and a biologically active fragment of any of the foregoing proteins.

The term “protein of interest (POI)” as used herein refers to a protein that is produced by means of recombinant technology in a host cell. More specifically, the protein may either be a polypeptide not naturally occurring in the host cell, i.e. a heterologous protein, or else may be native to the host cell, i.e. a homologous protein to the host cell, but is produced, for example, by transformation with a self replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g. of the promoter sequence.

The POI can be any eukaryotic or prokaryotic protein. The protein can be a naturally secreted protein or an intracellular protein, i.e. a protein which is not naturally secreted. The present invention also includes biologically active fragments of naturally secreted or not naturally secreted proteins.

A secreted POI referred to herein may be but is not limited to a protein suitable as a biopharmaceutical substance like an antibody or antibody fragment, growth factor, hormone, enzyme, vaccine, or a protein which can be used for industrial application like e.g. an enzyme.

A intracellular POI referred to herein may be but is not limited to a helper factor for protein secretion, or an enzyme used for metabolic engineering purposes.

In another embodiment, the POI is a eukaryotic protein or a biologically active fragment thereof, preferably an immunoglobulin or an immunoglobulin fragment such as a Fc fragment or a Fab fragment. Most preferably, the POI is a Fab fragment of the monoclonal anti-HIV1 antibody 2F5.

In general, the proteins of interest referred to herein may be produced by methods of recombinant expression well known to a person skilled in the art.

It is understood that the methods disclosed herein may further include cultivating said recombinant host cells under conditions permitting the expression of the POI. A secreted, recombinantly produced POI can then be isolated from the cell culture medium and further purified by techniques well known to a person skilled in the art.

As used herein, a “biologically active fragment” of a protein shall mean a fragment of a protein that exerts a biological effect similar or comparable to the full length protein. Such fragments can be produced e.g. by amino- and carboxy-terminal deletions as well as by internal deletions.

In general, the host cell from which the proteins are secreted can be any eukaryotic cell suitable for recombinant expression of a POI.

In a preferred embodiment, the invention relates to such a method, wherein the host cell is a fungal cell, e.g. a yeast cell, or a higher eukaryotic cell, e.g. a mammalian cell or a plant cell.

Examples of yeast cells include but are not limited to the Saccharomyces genus (e.g. Saccharomyces cerevisiae), the Komagataella genus (Komagataella pastoris, Komagataella pseudopastoris or Komagataella phaffii), Pichia methanolica, Hansenula polymorpha or Kluyveromyces lactis.

In a preferred embodiment the invention relates to a method, wherein the yeast cell is a cell of the Komagataella genus, in particular a cell of a strain of Komagataella pastoris, Komagataella pseudopastoris or Komagataella phaffii.

The former species Pichia pastoris has been divided and renamed to Komagataella pastoris and Komagataella phaffii (Kurtzman, 2005). Therefore Pichia pastoris is synonymous for both Komagataella pastoris and Komagataella phaffii.

The nucleotide sequences encoding the proteins that increase protein secretion can be obtained from a variety of sources. Said proteins may be involved in the eukaryotic protein secretory pathway.

In one aspect the invention relates to such a method, wherein at least one recombinant nucleotide sequence encoding a protein that increases protein secretion is a yeast nucleotide sequence, preferably but not limited to a nucleotide sequence of the yeast species Saccharomyces cerevisiae or Pichia pastoris. Also, homologous nucleotide sequences from other suitable yeasts or other fungi or from other organisms such as vertebrates can be used.

The term “homologous nucleotide sequences” as used herein refers to nucleotide sequences which are related but not identical in their nucleotide sequence with the contemplated nucleotide sequence, and perform essentially the same function.

In a further aspect the invention relates to such a method, wherein at least one recombinant nucleotide sequence encoding a protein that increases protein secretion is obtained from Saccharomyces cerevisiae and is identical with or corresponds to and has the functional characteristics of a sequence selected from the group consisting of SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40 and SEQ ID NO 41.

As used herein, the term “nucleotide sequence that corresponds to and has the functional characteristics of” is meant to encompass variations in its nucleotide composition including variations due to the degeneracy of the genetic code, whereby the nucleotide sequence performs essentially the same function.

By screening a P. pastoris genome database (ERGO™, IG-66, Integrated Genomics) with the nucleotide sequences of the secretion helper factors isolated from Saccharomyces cerevisiae homologous nucleotide sequences in Pichia pastoris have been identified. Preliminary experimental results indicate that these homologous nucleotide sequences isolated from Pichia pastoris show similar effects on protein secretion from a host cell when compared to the corresponding nucleotide sequences isolated from Saccharomyces cerevisiae.

In a further aspect the invention relates to such a method, wherein at least one recombinant nucleotide sequence encoding a protein that increases protein secretion is obtained from Pichia pastoris and is identical with or corresponds to and has the functional characteristics of a sequence selected from the group consisting of SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50 and SEQ ID NO 51.

In a further aspect the invention relates to such a method, wherein the recombinant nucleotide sequence encoding the POI is provided on a plasmid suitable for integration into the genome of the host cell, in a single copy or in multiple copies per cell. The recombinant nucleotide sequence encoding the POI may also be provided on an autonomously replicating plasmid in a single copy or in multiple copies per cell.

Alternatively, the recombinant nucleotide sequence encoding the POI and the recombinant nucleotide sequence encoding a protein that increases protein secretion are present on the same plasmid in single copy or multiple copies per cell.

The terms “plasmid” and “vector” as used herein include autonomously replicating nucleotide sequences as well as genome integrating nucleotide sequences.

In a further aspect, the invention relates to such a method, wherein the plasmid is a eukaryotic expression vector, preferably a yeast expression vector.

“Expression vectors” as used herein are defined as DNA sequences that are required for the transcription of cloned recombinant nucleotide sequences, i.e. of recombinant genes and the translation of their mRNA in a suitable host organism. Such expression vectors usually comprise an origin for autonomous replication in the host cells, selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together.

The term “operably linked” as used herein refers to the association of nucleotide sequences on a single nucleic acid molecule, e.g. a vector, in a way such that the function of one or more nucleotide sequences is affected by at least one other nucleotide sequence present on said nucleic acid molecule. For example, a promoter is operably linked with a coding sequence of a recombinant gene when it is capable of effecting the expression of that coding sequence.

Expression vectors may include but are not limited to cloning vectors, modified cloning vectors and specifically designed plasmids. The expression vector of the invention may be any expression vector suitable for expression of a recombinant gene in a host cell and is selected depending on the host organism.

In another aspect the invention relates to such a method, wherein the expression vector comprises a secretion leader sequence effective to cause secretion of the POI from the host cell.

The presence of such a secretion leader sequence in the expression vector is required when the POI intended for recombinant expression and secretion is a protein which is not naturally secreted and therefore lacks a natural secretion leader sequence, or its nucleotide sequence has been cloned without its natural secretion leader sequence. In general, any secretion leader sequence effective to cause secretion of the POI from the host cell may be used in the present invention. The secretion leader sequence may originate from yeast source, e.g. from yeast α-factor such as MFα of Saccharomyces cerevisiae, or yeast phosphatase, from mammalian or plant source, or others. The selection of the appropriate secretion leader sequence is apparent to a skilled person.

Alternatively, the secretion leader sequence can be fused to the nucleotide sequence encoding a POI intended for recombinant expression by conventional cloning techniques known to a skilled person prior to cloning of the nucleotide sequence in the expression vector or the nucleotide sequence encoding a POI comprising a natural secretion leader sequence is cloned in the expression vector. In these cases the presence of a secretion leader sequence in the expression vector is not required.

To allow expression of a recombinant nucleotide sequence in a host cell the expression vector has to provide the recombinant nucleotide sequence with a functional promoter adjacent to the 5′ end of the coding sequence. The transcription is thereby regulated and initiated by this promoter sequence.

In a further aspect the invention relates to such a method, wherein the expression vector comprises a promoter sequence effective to control expression of the POI in the host cell.

“Promoter sequence” as used herein refers to a DNA sequence capable of controlling the expression of a coding sequence or functional RNA.

Suitable promoter sequences for use with yeast host cells may include but are not limited to promoters obtained from genes that code for metabolic enzymes which are known to be present at high concentration in the cell, e.g. glycolytic enzymes like triosephosphate isomerase (TPI), phosphoglycerate kinase (PGK), glyceraldehyde-3-phosphate dehydrogenase (GAPDH), alcohol oxidase (AOX), lactase (LAC) and galactosidase (GAL).

Suitable promoter sequences for use with mammalian host cells may include but are not limited to promoters obtained from the genomes of viruses, heterologous mammalian promoters, e.g. the actin promoter or an immunoglobulin promoter, and heat shock protein promoters.

In order to identify novel promoter sequences for use in yeast host cells, preferably for use in a strain of the Komagataella genus, in particular for use in a strain of K. pastoris, K. pseudopastoris or K. phaffii for recombinant expression of a POI, the data derived from the DNA microarray hybridisation described in Example 1 were evaluated in a specific manner.

The promoter sequences of the 23 most interesting genes identified by this analysis (up to 1000 bp of the 5′-region of the respective genes) were amplified from P. pastoris by PCR and cloned into a P. pastoris expression vector, which additionally carries an enhanced green fluorescent protein (eGFP) as a reporter gene. To test the properties of the different promoters, i.e. the promoter activity, the 25 vectors (including two control vectors) were subsequently transformed into a P. pastoris strain. The clones were cultivated under different culturing conditions and the amount of recombinant eGFP was quantified using flow cytometer analysis. A comparative analysis of the well established yeast promoter of GAP and the 23 promoter sequences is provided in Example 5.

The term “promoter activity” as used herein refers to an assessment of the transcriptional efficiency of a promoter. This may be determined directly by measurement of the amount of mRNA transcription from the promoter, e.g. by Northern Blotting or indirectly by measurement of the amount of gene product expressed from the promoter.

It was surprisingly found that a 1000 bp fragment from the 5′-non coding region of the PET9 gene of P. pastoris results in real unexpected high expression levels of recombinant eGFP, ranging from about 700% to about 1600% of the promoter activity of the GAP promoter, depending on the carbon source during cultivation, under the experimental conditions as described in Example 5.

PET9 is known from S. cerevisiae as a major ADP/ATP carrier of the mitochondrial inner membrane, which exchanges cytosolic ADP for mitochondrial synthesized ATP.

In another aspect the invention relates to a method of increasing the secretion of a POI from a eukaryotic cell, wherein the nucleotide sequence encoding the POI is controlled by a promoter sequence which is a 1000 bp fragment from the 5′-non coding region of the PET9 gene of Pichia pastoris corresponding to SEQ ID NO 125, or a functionally equivalent variant thereof and the host cell is a cell of the genus Komagataella, in particular a cell of a strain of K. pastoris, K. pseudopastoris or K. phaffii.

In another aspect the invention relates to the use of a nucleotide sequence isolated from Saccharomyces cerevisiae and encoding a protein that increases protein secretion and being selected from the group consisting of BMH2, BFR2, C0G6, C0Y1, CUP5, IMH1, KIN2, SEC31, SSA4, SSE1, and a biologically active fragment of any of the foregoing proteins, as a secretion enhancer, particularly as an enhancer of the secretion of a POI from a eukaryotic cell, preferably in a yeast cell and most preferred in a cell of a strain of K. pastoris, K. pseudopastoris or K. phaffii.

In a further aspect the invention relates to such a use wherein the nucleotide sequence encoding a protein that increases protein secretion is identical with or corresponds to and has the functional characteristics of a sequence selected from the group consisting of SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40 and SEQ ID NO 41.

In another aspect the invention relates to the use of a nucleotide sequence isolated from Pichia pastoris and encoding a protein that increases protein secretion and being selected from the group consisting of BMH2, BFR2, C0G6, C0Y1, CUP5, IMH1, KIN2, SEC31, SSA4, SSE1, and a biologically active fragment of any of the foregoing proteins, as a secretion enhancer, particularly as an enhancer of the secretion of a POI from a eukaryotic cell, preferably in a yeast cell and most preferred in a cell of a strain of K. pastoris, K. pseudopastoris or K. phaffii.

In a further aspect the invention relates to such a use, wherein the nucleotide sequence encoding a protein that increases protein secretion is identical with or corresponds to and has the functional characteristics of a sequence selected from the group consisting of SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50 and SEQ ID NO 51.

SSA4 is a member of the HSP70 family of molecular chaperones. SSA4 is participating in the SRP-dependent targeting of protein to the ER membrane prior to the cotranslational translocation of the protein into the ER-lumen, and is induced upon stress response.

The chaperonines of the SSE/HSP110 subclass of the HSP70 family, that are encoded by SSE1 and SSE2, assist in folding by binding to nascent peptides and holding them in a folding-competent state, however, they can not actively promote folding reactions. On the basis of their “holdase” activity, interactions to chaperones such as Ssa1p and Ssb1p of the HSP70 family as well as to the HSP90 complex seem plausible.

Sec31p is an essential phosphoprotein component of the coat protein complex II (COPII) of secretory pathway vesicles, in complex with Sec13p.

Growth defects due to mutations in either Sec13 or Sec23 (as well as Sec16 and Ypt1) can be overcome by overexpression of the essential S. cerevisiae gene BFR2. It has been isolated as a multi-copy suppressor of the drug Brefeldin A, a fungal metabolite that perturbs the protein flux into the Golgi and the structure of the Golgi apparatus itself.

14-3-3 proteins, encoded by BMH1 and BMH2, were identified to participate in multiple steps of vesicular trafficking, especially in protein exit from the ER, forward trafficking of multimeric cell surface membrane proteins as well as in retrograde transportation within the Golgi apparatus.

C0G6 belongs to one of eight genes coding for the Conserved Oligomeric Golgi (COG) complex, an eight-subunit peripheral Golgi protein, that is engaged in membrane trafficking and synthesis of glycoconjugates. Moreover, the COG complex is not only necessary for maintaining normal Golgi structure and function, but is also directly involved in retrograde vesicular transport within the Golgi apparatus.

The molecular function of Coy1, a protein identified by similarity to mammalian CASP, is not established yet, but is seems to be playing a role in Golgi vesicle transport through interaction with Gos1. Gos1 is a SNARE (soluble N-ethylmaleimide-sensitive factor attachment protein receptor) protein commonly used as marker of later compartments of the Golgi in S. cerevisiae.

The product of the IMH1/SYS3 gene is a member of the peripheral membrane Golgins involved in vesicular transport between the late Golgi and a prevacuolar, endosome-like compartment. Imh1 is recruited by to the Golgi by the two ARF-like (ARL) GTPases, Arl1p and Arl3p.

Kin2, and the closely related Kin1, are two serine/threonine protein kinases localized at the cytoplasmic side of the plasma membrane. The catalytic activity of Kin2 is essential for its function in regulation of exocytosis by phosphorylation of the plasma membrane t-SNARE Sec9, a protein acting at the final step of exocytosis. Genetic analysis indicates that the KIN kinases act downstream of the Exocyst, the vesicle tethering factor at the site of exocytosis, and its regulator Sec4 (GTP binding protein of the Ras family).

CUP5 encodes the c subunit of the yeast vacuolar (H)-ATPase (V-ATPase) Vₒ domain, belonging to a family of ATP-dependent proton pumps that acidify the yeast central vacuole. The Vₒ domain is an integral membrane structure of five subunits responsible for transporting protons across the membrane.

Assembling of the Vₒ domain is not possible in the absence of Cup5. V-ATPase function is important for many processes including endocytosis, protein degradation and coupled transport across the vacuolar membrane.

Additionally, a role for V-ATPase in detoxification of copper, iron metabolism and mitochondrial function was reported.

In another aspect the invention relates to a nucleotide sequence encoding a protein that increases protein secretion from a host cell, wherein the nucleotide sequence is isolated from Pichia pastoris and is identical with or corresponds to and has the functional characteristics of a sequence selected from the group consisting of a nucleotide sequence encoding the protein BMH2 (SEQ ID NO 42), a nucleotide sequence encoding the protein BFR2 (SEQ ID NO 43), a nucleotide sequence encoding the protein C0G6 (SEQ ID NO 44), a nucleotide sequence encoding the protein C0Y1 (SEQ ID NO 45), a nucleotide sequence encoding the protein CUP5 (SEQ ID NO 46), a nucleotide sequence encoding the protein IMH1 (SEQ ID NO 47), a nucleotide sequence encoding the protein KIN2 (SEQ ID NO 48), a nucleotide sequence encoding the protein SEC31 (SEQ ID NO 49), a nucleotide sequence encoding the protein SSA4 (SEQ ID NO 50) and a nucleotide sequence encoding the protein SSE1 (SEQ ID NO 51).

In a further aspect the invention relates to a yeast promoter sequence being a 1000 bp fragment from the 5′-non coding region of the PET9 gene corresponding to SEQ ID NO 125, or a functionally equivalent variant thereof and being isolated from Pichia pastoris.

It should be recognized that promoter sequences of various diminishing length may have identical promoter activity and should be therefore also included in the present invention, since the exact boundaries of the regulatory sequence of the 5′-non coding region of the PET9 gene have not been defined.

Therefore the term “functionally equivalent variant” of a promoter sequence as used herein means a nucleotide sequence resulting from modification of this nucleotide sequence by insertion, deletion or substitution of one or more nucleotides within the sequence or at either or both of the distal ends of the sequence, and which modification does not affect (in particular impair) the promoter activity of this nucleotide sequence.

In a further aspect the invention relates to such a yeast promoter sequence which has, under comparable conditions, improved properties for expression of a POI in yeast, preferably in a strain of the genus Komagataella, in particular in a strain of Komagataella pastoris, Komagataella pseudopastoris or Komagataella phaffii, relative to a yeast promoter known in the art, in particular relative to a GAP promoter isolated from Pichia pastoris.

In a further aspect the invention relates to such a yeast promoter sequence, having, under comparable conditions, at least the same, or at least about a 1.5-fold, or at least about 2-fold, or at least about a 4-fold, 7-fold, 10-fold, or at least up to about a 15-fold promoter activity relative to a GAP promoter isolated from Pichia pastoris.

It is desirable to have an expression system for recombinant expression of a nucleotide sequence in a host organism, in particular in a yeast host, more particular in a strain of the genus Komagataella, which offers the opportunity to easily change the different parts of the vector, like the selection marker, e.g. a resistance for zeocin, kanamycin/geneticin, hygromycin and others, the promoter or the transcription terminator. It would be also advantageous if the vector could either be integrated into the genome of the host (using homologous integration sequences) or located episomally by exchanging a part of the vector which is not important for heterologous gene expression.

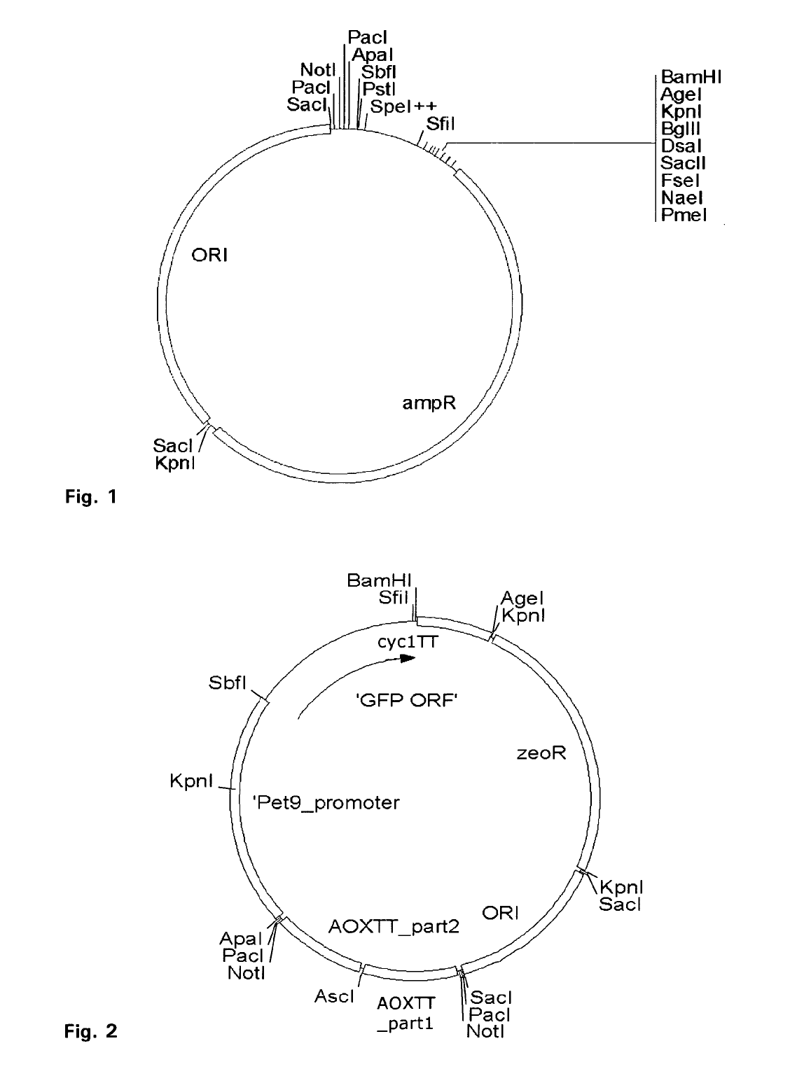

For construction of a novel vector system pPuzzle which provides the above mentioned advantages, in a first step a vector backbone of pPuzzle was generated carrying an origin of replication and a selection marker for Escherichia coli (E. coli), which enables amplification of the vector backbone in E.coli. In addition, the vector backbone of pPuzzle comprises a multiple cloning site (see FIG. 1 and Example 3).

In a second step the pPuzzle expression vector carrying a eukaryotic selection marker, a promoter for recombinant expression of a heterologous or homologous nucleotide sequence, a transcription terminator and optionally sequences for homologous integration of the vector in the host genome was constructed (see Example 4). The selection of the promoter sequence and the selection marker depends on the host organism which is used for recombinant expression of a nucleotide sequence. The transcription terminator can be, in principle, each functional transcription terminator and is in particular the transcription terminator of the cytochrome c gene from S. cerevisiae. Further, the presence of homologous integration sequences depends on whether the nucleotide sequence is intended to be integrated in the genome of the host organism or not. Since the selection marker, the promoter sequence and the homologous integration sequences are flanked by unique restriction enzyme cleavage sites they can easily be exchanged, i.e. cut out and substituted, whereby the vector can be altered or adapted to a selected host organism in a simple and efficient way.

In detail, the selection marker is cloned in a unique KpnI restriction site, the homologous integration sequences are cloned in a unique NotI restriction site, the promoter is cloned by using the ApaI and the SbfI/AarI restriction site and the nucleotide sequence encoding a POI is cloned in the MCS (multiple cloning site) using the restriction sites SbfI and SfII.

In a further aspect the invention relates to an eukaryotic expression vector based on the pPuzzle backbone further comprising the following components operably linked to each other:
a recombinant nucleotide sequence encoding a POI, optionally linked to a leader sequence effective to cause secretion of the POI from the host cell;
a promoter effective to control protein expression in a host cell;
a transcription terminator;
a selection marker;
either homologous integration sequences or autonomous replication sequences,

wherein the promoter is a 1000 bp fragment from the 5′-non coding region of the PET9 gene of Pichia pastoris (SEQ ID NO 125), or a functionally equivalent variant thereof, the transcription terminator is the transcription terminator of the cytochrome c gene from S. cerevisiae, the selection marker is a zeocin resistance gene and the host cell is a yeast cell, preferably a cell of a strain of the genus Komagataella, in particular a cell of a strain of Komagataella pastoris, Komagataella pseudopastoris or Komagataella phaffii.

A detailed description of the procedure for the construction of such a vector, which additionally contains an enhanced green fluorescent protein eGFP as a reporter gene (pPuzzle_zeoR_Ppet9_eGFP_AOXTT) is found in Examples 3 to 5 and in FIG. 2.

It is understood that any heterologous or homologous nucleotide sequence intended for recombinant expression in a host cell can be used in the position of eGFP.

In another aspect the invention relates to the use of such a eukaryotic expression vector for recombinant expression of a POI in a host cell.

Depending on the problem to be solved it can be desirable to either have a strong expression of a protein of interest in a host cell (e.g. for recombinant production of a POI in a host cell) or to have a weak or reduced expression of a protein of interest in a host cell (e.g. when analysing the molecular function of a POI in a host cell).

Particularly, in case of the analysis of the molecular function of a cellular POI or in case of a POI intended for metabolic engineering applications, which protein shall not be secreted, but develop its activity within a desired compartment of the cell, it would be attractive being able to regulate the expression level of this protein of interest via the promoter activity. It can be desirable to either have a strong expression of the POI (comparable to or stronger as from the GAP promoter) or to have a weak or reduced expression of the POI (less than from the GAP promoter). It is therefore useful to have a selection of different promoter sequences suitable for recombinant expression of a heterologous or homologous nucleotide sequence in a host organism, in particular in a yeast host, more particular in a strain of the genus Komagataella, in particular in a cell of a strain of Komagataella pastoris, Komagataella pseudopastoris or Komagataella phaffii, having different promoter activities under comparable cell culture conditions, varying from strong promoter activity to weak or reduced promoter activity as compared to the GAP promoter. This allows to regulate the expression level of a protein of interest by selection of a suitable promoter sequence according to the experimental situation.

From the comparative analysis of promoter sequences as described in Example 5, i.e. from the analysis of the promoter activity, several promoter sequences with different promoter activities, ranging from 0% to about 135% of the promoter activity of a GAP promoter isolated from Pichia pastoris, under the experimental conditions as described in Example 5, have been found.

A summary of the promoter activities of the yeast promoter sequences tested in Example 5 (determined by measurement of the relative expression level in % of the reporter gene product eGFP and standardisation on eGFP expression under the GAP promoter) is found in Table 8.

In detail, a 1000 bp fragment from the 5′-non coding region of the GND1 gene had, under the experimental conditions of Example 5, a promoter activity ranging from 0% to about 67% of the promoter activity of the GAP promoter. A 1000 bp fragment from the 5′-non coding region of the GPM1 gene had, under the experimental conditions of Example 5, a promoter activity ranging from about 19% to about 41% of the promoter activity of the GAP promoter. A 1000 bp fragment from the 5′-non coding region of the HSP90 gene had, under the experimental conditions of Example 5, a promoter activity ranging from about 6% to about 81% of the promoter activity of the GAP promoter. A 1000 bp fragment from the 5′-non coding region of the KAR2 gene had, under the experimental conditions of Example 5, a promoter activity ranging from about 11% to about 135% of the promoter activity of the GAP promoter. A 1000 bp fragment from the 5′-non coding region of the MCM1 gene had, under the experimental conditions of Example 5, a promoter activity ranging from 0% to about 6% of the promoter activity of the GAP promoter. A 1000 bp fragment from the 5′-non coding region of the RAD2 gene had, under the experimental conditions of Example 5, a promoter activity ranging from 0% to about 5% of the promoter activity of the GAP promoter. A 1000 bp fragment from the 5′-non coding region of the RPS2 gene had, under the experimental conditions of Example 5, a promoter activity ranging from 0% to about 12% of the promoter activity of the GAP promoter. A 1000 bp fragment from the 5′-non coding region of the RPS31 gene had, under the experimental conditions of Example 5, a promoter activity ranging from 0% to about 8% of the promoter activity of the GAP promoter. A 1000 bp fragment from the 5′-non coding region of the SSA1 gene had, under the experimental conditions of Example 5, a promoter activity ranging from 0% to about 30% of the promoter activity of the GAP promoter. A 1000 bp fragment from the 5′-non coding region of the THI3 gene had, under the experimental conditions of Example 5, a promoter activity ranging from 0% to about 42% of the promoter activity of the GAP promoter. A 1000 bp fragment from the 5′-non coding region of the TPI1 gene had, under the experimental conditions of Example 5, a promoter activity ranging from 0% to about 92% of the promoter activity of the GAP promoter. A 1000 bp fragment from the 5′-non coding region of the UBI4 gene had, under the experimental conditions of Example 5, a promoter activity ranging from 0% to about 4% of the promoter activity of the GAP promoter. A 1000 bp fragment from the 5′-non coding region of the ENO1 gene had, under the experimental conditions of Example 5, a promoter activity ranging from 17% to about 47% of the promoter activity of the GAP promoter. A 1000 bp fragment from the 5′-non coding region of the RPS7A gene had, under the experimental conditions of Example 5, a promoter activity ranging from 1% to about 18% of the promoter activity of the GAP promoter. A 1000 bp fragment from the 5′-non coding region of the RPL1 gene had, under the experimental conditions of Example 5, a promoter activity ranging from 0% to about 11% of the promoter activity of the GAP promoter. A 1000 bp fragment from the 5′-non coding region of the TKL1 gene had, under the experimental conditions of Example 5, a promoter activity ranging from 0% to about 9% of the promoter activity of the GAP promoter. A 1000 bp fragment from the 5′-non coding region of the PIS1 gene had, under the experimental conditions of Example 5, a promoter activity ranging from 0% to about 7% of the promoter activity of the GAP promoter. A 1000 bp fragment from the 5′-non coding region of the FET3 gene had, under the experimental conditions of Example 5, a promoter activity ranging from 0% to about 7% of the promoter activity of the GAP promoter. A 1000 bp fragment from the 5′-non coding region of the FTR1 gene had, under the experimental conditions of Example 5, a promoter activity ranging from 0% to about 6% of the promoter activity of the GAP promoter. A 1000 by fragment from the 5′-non coding region of the NMT1 gene had, under the experimental conditions of Example 5, a promoter activity ranging from 0% to about 5% of the promoter activity of the GAP promoter. A 1000 by fragment from the 5′-non coding region of the PHO8 gene had, under the experimental conditions of Example 5, a promoter activity ranging from 0% to about 6% of the promoter activity of the GAP promoter. A 1000 bp fragment from the 5′-non coding region of the FET3 precursor (FET3pre) gene had, under the experimental conditions of Example 5, a promoter activity ranging from 0% to about 7% of the promoter activity of the GAP promoter.

In another aspect the invention relates to a yeast promoter sequence being isolated from Pichia pastoris and being identical with or corresponding to and having the functional characteristics of a sequence selected from the group consisting of a 1000 bp fragment from the 5′-non coding region of the GND1 gene (SEQ ID NO 126), a 1000 bp fragment from the 5′-non coding region of the GPM1 gene (SEQ ID NO 127), a 1000 bp fragment from the 5′-non coding region of the HSP90 gene (SEQ ID NO 128), a 1000 bp fragment from the 5′-non coding region of the KAR2 gene (SEQ ID NO 129), a 1000 bp fragment from the 5′-non coding region of the MCM1 gene (SEQ ID NO 130), a 1000 bp fragment from the 5′-non coding region of the RAD2 gene (SEQ ID NO 131), a 1000 by fragment from the 5′-non coding region of the RPS2 gene (SEQ ID NO 132), a 1000 bp fragment from the 5′-non coding region of the RPS31 gene (SEQ ID NO 133), a 1000 bp fragment from the 5′-non coding region of the SSA1 gene (SEQ ID NO 134), a 1000 bp fragment from the 5′-non coding region of the THIS gene (SEQ ID NO 135), a 1000 bp fragment from the 5′-non coding region of the TPI1 gene (SEQ ID NO 136), a 1000 bp fragment from the 5′-non coding region of the UBI4 gene (SEQ ID NO 137), a 1000 bp fragment from the 5′-non coding region of the ENO1 gene (SEQ ID NO 138), a 1000 bp fragment from the 5′-non coding region of the RPS7A gene (SEQ ID NO 139), a 1000 bp fragment from the 5′-non coding region of the RPL1 gene (SEQ ID NO 140), a 1000 bp fragment from the 5′-non coding region of the TKL1 gene (SEQ ID NO 141), a 1000 bp fragment from the 5′-non coding region of the PIS1 gene (SEQ ID NO 142), a 1000 bp fragment from the 5′-non coding region of the FET3 gene (SEQ ID NO 143), a 1000 bp fragment from the 5′-non coding region of the FTR1 gene (SEQ ID NO 144), a 1000 bp fragment from the 5′-non coding region of the NMT1 gene (SEQ ID NO 145), a 1000 bp fragment from the 5′-non coding region of the PHO8 gene (SEQ ID NO 146), and a 1000 bp fragment from the 5′-non coding region of the FET3 precursor (FET3pre) gene (SEQ ID NO 147), or a functionally equivalent variant of any of the foregoing sequences.

Enolase 1 (ENO1) is a phosphopyruvate hydratase that catalyzes the conversion of 2-phosphoglycerate to phosphoenolpyruvate during glycolysis and the reverse reaction during gluconeogenesis.

Triose phosphate isomerase (TPI1) is an abundant glycolytic enzyme. It catalyzes the interconversion of glyceraldehyde-3-phosphate and dihydroxyacetone phosphate during glycolysis.

THI3 is a probable decarboxylase, required for expression of enzymes involved in thiamine biosynthesis and may have a role in catabolism of amino acids to long-chain and complex alcohols.

SSA1 is an ATPase involved in protein folding and nuclear localization signal (NLS)-directed nuclear transport. SSA1 is member of heat shock protein 70 (HSP70) family.

RPS7A is a protein component of the small (40S) ribosomal subunit.

6-Phosphogluconate dehydrogenase (GND1) catalyzes an NADPH regenerating reaction in the pentose phosphate pathway and is required for growth on D-glucono-delta-lactone and adaptation to oxidative stress.

GPM1 encodes the phosphoglycerate mutase, which is a tetrameric enzyme responsible for the conversion of 3-phospholycerate to 2-phosphoglycerate during glycolysis, and the reverse reaction during gluconeogenesis.

Transketolase (TKL1) catalyzes conversion of xylulose-5-phosphate and ribose-5-phosphate to sedoheptulose-7-phosphate and glyceraldehyde-3-phosphate in the pentose phosphate pathway and is needed for synthesis of aromatic amino acids.

Heat Shock Protein 90 (HSP90) is a cytoplasmic chaperone (Hsp90 family).

RPS2 is a protein component of small ribosomal(40S) subunit.

RPS31 is a fusion protein that is cleaved to yield a ribosomal protein of the small (40S) subunit and ubiquitin.

RPL1A is a protein component of the large ribosomal (60S) subunit.

The phosphatidylinositol synthase PIS1 is required for biosynthesis of phosphatidylinositol, which is a precursor for polyphosphoinositides, sphingolipids, and glycolipid anchors for some of the plasma membrane proteins.

Ferro-O₂-oxidoreductase (FET3) belongs to class of integral membrane multicopper oxidases and is required for high-affinity iron uptake and involved in mediating resistance to copper ion toxicity, FET3pre its precursor.

The high affinity iron permease (FTR1) is involved in the transport of iron across the plasma membrane and forms complex with Fet3p.

PHO8 is a repressible alkaline phosphatase.

N-myristoyl transferase NMT1 catalyzes the cotranslational, covalent attachment of myristic acid to the N-terminal glycine residue of several proteins involved in cellular growth and signal transduction.

The transcription factor MCM1 is involved in cell-type-specific transcription and pheromone response.

Ubiquitin (UBI4) becomes conjugated to proteins, marking them for selective degradation via the ubiquitin-26S proteasome system.

RAD2, a single-stranded DNA endonuclease, cleaves single-stranded DNA during nucleotide excision repair to excise damaged DNA.

In a further aspect the invention relates to a eukaryotic expression vector based on the pPuzzle backbone further comprising the following components operably linked to each other:
a recombinant nucleotide sequence encoding a POI, optionally linked to a leader sequence effective to cause secretion of the POI from the host cell;
a promoter effective to control protein expression in a host cell;
a transcription terminator;
a selection marker;
either homologous integration sequences or autonomous replication sequences,

wherein the promoter is a yeast promoter sequence isolated from Pichia pastoris and is identical with or corresponds to and has the functional characteristics of a sequence selected from the group consisting of SEQ ID NO 125, SEQ ID NO 126, SEQ ID NO 127, SEQ ID NO 128, SEQ ID NO 129, SEQ ID NO 130, SEQ ID NO 131, SEQ ID NO 132, SEQ ID NO 133, SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 143, SEQ ID NO 144, SEQ ID NO 145, SEQ ID NO 146 and SEQ ID NO 147, or a functionally equivalent variant of any of the foregoing sequences, and the host cell is a yeast cell, preferably a cell of a strain of the genus Komagataella, in particular a cell of a strain of Komagataella pastoris, Komagataella pseudopastoris or Komagataella phaffii.

In another aspect the invention relates to the use of such a eukaryotic expression vector for recombinant expression of a POI in a host cell.

In case, that the POI is a cellular protein intended for metabolic engineering applications, i.e. for expression and developing its activity within a desired compartment of a host cell the POI may be expressed from a eukaryotic expression vector based on the pPuzzle backbone without a leader sequence effective to cause secretion of the POI from the host cell.

If the cellular POI is a homologous protein to the host cell, i.e. a protein which is naturally occurring in the host cell, the expression of the POI in the host cell may be modulated by the exchange of its native promoter sequence with a yeast promoter sequence isolated from Pichia pastoris and being identical with or corresponding to and having the functional characteristics of a sequence selected from the group consisting of SEQ ID NO 125, SEQ ID NO 126, SEQ ID NO 127, SEQ ID NO 128, SEQ ID NO 129, SEQ ID NO 130, SEQ ID NO 131, SEQ ID NO 132, SEQ ID NO 133, SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 143, SEQ ID NO 144, SEQ ID NO 145, SEQ ID NO 146 and SEQ ID NO 147 or a functionally equivalent variant of any of the foregoing sequences.

This purpose may be achieved e.g. by transformation of a host cell with a recombinant DNA molecule comprising homologous sequences of the target gene to allow site specific recombination, the desired yeast promoter sequence and a selective marker suitable for the host cell. The site specific recombination shall take place in order to operably link the yeast promoter sequence with the nucleotide sequence encoding the POI. This results in the expression of the POI from the yeast promoter sequence instead of from the native promoter sequence.

Depending on the problem to be solved the selected yeast promoter may have either an increased promoter activity relative to the native promoter sequence leading to an increased expression of a POI in the host cell or may have a decreased promoter activity relative to the native promoter sequence leading to a reduced expression of a POI in the host cell.

In another aspect the invention relates to the use of a yeast promoter sequence being isolated from Pichia pastoris and being identical with or corresponding to and having the functional characteristics of a sequence selected from the group consisting of SEQ ID NO 125, SEQ ID NO 126, SEQ ID NO 127, SEQ ID NO 128, SEQ ID NO 129, SEQ ID NO 130, SEQ ID NO 131, SEQ ID NO 132, SEQ ID NO 133, SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 143, SEQ ID NO 144, SEQ ID NO 145, SEQ ID NO 146 and SEQ ID NO 147 or a functionally equivalent variant of any of the foregoing sequences for modulation of the expression of a homologous POI in a host cell.

In another aspect the invention relates to such a use, wherein the yeast promoter sequence has an increased promoter activity relative to the native promoter sequence of the POI.

In another aspect the invention relates to such a use, wherein the yeast promoter sequence has a decreased promoter activity relative to the native promoter sequence of the POI.

In order that the invention described herein may be more fully understood, the following examples are set forth. The examples are for illustrative purposes only and are not to be construed as limiting this invention in any respect. It is further understood that the present invention shall also comprise variations of the expressly disclosed embodiments to an extent as would be contemplated by a person of ordinary skill in the art.

### Examples

Examples 1 and 2 below illustrate the materials and methods used to investigate the effect of co-expression of different proteins involved in the eukaryotic secretion pathway (secretion helper factors) on the yield of a secreted heterologous protein of interest, i.e. on the secretion of the Fab fragment of the monoclonal anti-HIV1 antibody 2F5 in P. pastoris.

### Example 1

### Identification and Cloning of Several Secretion Helper Factors from Saccharomyces cerevisiae

In order to identify genes and their respective proteins which play a potential role during protein production, e.g. in the protein secretory pathway of P. pastoris the gene expression pattern of a P. pastoris strain containing the gene for human trypsinogen 1 was compared before and after induction of heterologous protein production (induction was done by a switch from glycerol to methanol as the sole carbon source), i.e. of trypsinogen production by microarray analysis.

As the genome sequence of P. pastoris has not been published and not many genes are characterized for P. pastoris DNA microarrays of S. cerevisiae were used for heterologous hybridization with P. pastoris cDNA.

The experimental procedure of the microarray hybridisation and the evaluation of the obtained data was carried out as described in Sauer et al. (2004). Further details are found below.

a) Strain:

The expression strain was P. pastoris strain X33 (Invitrogen), a wild type strain which can grow on minimal media without supplements. The selection mechanism was based on the Zeocin™ resistance of the transformation vector. Transformation of the strain was carried out with a plasmid derived from pPICZαB (Invitrogen), containing the gene for human trypsinogen 1 (Hohenblum et al., 2003). pPICZαB utilises the AOX1 promoter of P. pastoris, which promoter is repressed by many carbon sources such as glucose, glycerol or ethanol but induced by the carbon source methanol, and the α-factor leader sequence of S. cerevisiae for product secretion. The selected strain was of the methanol utilisation positive (mut⁺) phenotype, which means that it is fully capable to metabolise methanol as the sole carbon source.

b) Cell Culture:

Fermentation of P. pastoris

Fed batch fermentations were performed with a MBR mini bioreactor with a final working volume of 2 l, essentially as described by Hohenblum et al. (2003).

The media were as follows:

PTM, Trace Salts Stock Solution Contained Per Litre

6.0 g CuSO₄.5H₂O, 0.08 g NaI, 3.0 g MnSO₄.H₂O, 0.2 g Na₂MoO₄.2H₂O, 0.02 g H₃BO₃, 0.5 g CoCl₂, 20.0 g ZnCl₂, 65.0 g FeSO₄.7H₂O, 0.2 g biotin and 5.0 ml H₂SO₄ (95%-98%). All chemicals for PTM, trace salts stock solution were from Riedel-de Haën, except for biotin (Sigma), and H₂SO₄ (Merck Eurolab).

Batch Medium Contained Per Litre

23.7 ml H₃PO₄ (85%), 0.6 g CaSO₄.2H₂O, 9.5 g K₂SO₄, 7.8 g MgSO₄.7H₂O, 2.6 g KOH, 40 g glycerol, 4.4 ml PTM, trace salts stock solution.

Glycerol Fed-Batch Solution Contained Per Litre

632 g glycerol (100%) and 12 ml PTM, trace salts stock solution.

Methanol Fed-Batch Solution Contained Per Litre

988 ml methanol (100%) and 12 ml PTM, trace salts stock solution.

The dissolved oxygen was controlled at DO=30% with the stirrer speed (600-1200 rpm). Aeration rate was 100 l h⁻¹ air, which was supplemented with oxygen (up to 25%) after the begin of the fed batch. The temperature was 25° C., and the pH was controlled with NH₃ (25%).

Before starting the fermentation, the pH of 1.2 l batch medium was set to 5.0 with NH₃ (25%). The batch phase of approximately 32 h was followed by a 4 h fed batch with glycerol medium (feed rate 15.6 ml h⁻¹), leading to a dry biomass concentration of approximately 40 g l⁻¹. Then, the feed with methanol medium was started with a feed rate of 6.4 ml h⁻¹. Methanol induces the production of the heterologous protein trypsinogen and serves as a carbon source at the same time. The fermentation was terminated 14 h after the methanol feed start. The pH was 5.0 during batch, and kept at 5.0 throughout the fermentation.

Samples were taken at the end of the glycerol fed batch phase (trypsinogen non-expressing cells) and at the end of the methanol fed batch phase (trypsinogen expressing cells), respectively. Cells were centrifuged to separate the cell culture supernatant, then the cell pellets were resuspended in 10× the volume of TRI-reagent (Sigma) and frozen.

c) RNA Isolation:

The samples were thawed on ice and after addition of acid washed glass beads the cells were homogenised in a Ribolyser (Hybaid Ltd.) for 2×20 sec, in between cooling on ice. After addition of chloroform, the samples were centrifuged and the total RNA was precipitated from the aqueous phase adding isopropanol. The pellet was washed 2× with 70% ethanol, dried and re-suspended in RNAse free water. mRNA was isolated using the MicroPoly(A)Purist mRNA purification Kit (Ambion) according to the manufacturers protocol.

d) Synthesis and Labelling of cDNA:

5 μg of mRNA and 0.5 μg of oligodT primer were mixed in 7 μl of water, incubated for 5 min at 70° C. and subsequently at 42° C. for about 3 min. The following components were added to 5 μl of said reaction mixture: 4 μl reaction buffer (5×) for SuperScript II reverse transcriptase (Invitrogen), 2 μl dTTP (2 mM), 2 μl dATP, dGTP, dCTP (5 mM), 2 μl DTT (100 mM), 2.5 μl RNasin (40 U, Promega) and either 2 μl FluoriLink Cy3-dUTP (1 mM) or 2 μl FluoriLink Cy5-dUTP (1 mM, Amersham Biosciences) respectively, and 1 μl SuperScript II reverse transcriptase (200 U, Invitrogen) to result in a total of 19.5 μl. The mixture was incubated for 1 h at 42° C. After addition of further 200 U SuperScript II reverse transcriptase the mixture was incubated for another 1 h at 42° C. 7 μl of 0.5 M NaOH/50 mM EDTA were added and the mixture was incubated at 70° C. for 15 min. The reaction mixture was neutralised by addition of 10 μl Tris-HCl pH 7.5 (1 M). The labelled cDNA was purified with Qiaquick purification columns (Qiagen) according to the manufacturer's protocol.

e) Chip Hybridisation and Set-Up of Microarrays:

The S. cerevisiae cDNA microarrays used for this study were Hyper Gene Yeast Chips from Hitachi Software Engineering Europe AG. According to the manufacturer, about 0.1 to 0.3 ng of PCR amplified cDNA (approximately 200 bp to 8000 bp) were spotted onto a poly-L-lysine coated glass slide and fixed by baking, succinic anhydride blocking and heat denaturation.

Labelled cDNA was resuspended in about 70 μl of 5×SSC/0.05% SDS, heat denatured at 95° C. for 3 min and cooled on ice. SDS crystals appearing were dissolved by short and slight warming and the mixture was gently applied to a Yeast Chip. The spotted area was covered with a cover glass and the chips were placed in an airtight container with a humidified atmosphere at 60° C. for 16 h.

The cover glasses were removed in 2×SSC/0.1% SDS and the chips were washed consecutively for 5-10 min each in 2×SSC/0.1% SDS, 0.5×SSC/0.1% SDS, and 0.2×SSC/0.1% SDS at RT. The chips were centrifuged at 600 rpm for 3 min in order to dry them. The washing conditions were chosen according to the manufacturer's manual.

Each sample (labelled cDNA from trypsinogen non-expressing cells and from trypsinogen expressing cells) was used for hybridisation of two parallel cDNA mircoarrays to test the reproducibility of the signals.

f) Data Acquisition and Statistical Evaluation of Microarray Data:

Images were scanned at a resolution of 50 μm with a G2565AA Microarray scanner (Agilent) and were imported into the GenePix Pro 4.1 (Axon Instruments) microarray analysis software. GenePix Pro 4.1 was used for the quantification of the spot intensities. Each appearing gene spot was averaged. The data set was then imported into GeneSpring 6.1 (Silicon Genetics) for further normalisation and data analysis.

All of the values of each channel on each chip were divided by their respective median for normalisation. Subsequently, the median intensity of all TE spots (spotted with buffer, no DNA) deduced from each value, and all spot values less than the standard deviation of said threshold values were considered to be not significant and were set to the value of the standard deviation. To determine induction or repression of gene activity, the normalised signals on each spot were compared, and all genes showing a signal difference exceeding the threshold (1.5 fold) on both parallel independent microarrays were judged as significantly regulated.

After determination of the the relative expression levels of all measured genes, the genes were ordered by the relative difference of their expression levels, and the 524 with the highest difference were considered for further analysis. As the DNA microarrays used for these experiments were derived from Saccharomyces cerevisiae gene sequences, only putative gene functions for P. pastoris can be assigned by the homology to S. cerevisiae. After ranking the 524 differentially regulated genes based on their putative intracellular localisation and function, and focusing on those being involved in secretion and/or general stress response, out of the 64 potentially interesting genes 15 were selected for further analysis: PDI1, CUP5, SSA4, BMH2, KIN2, KAR2, HAC1, ERO1, SSE1, BFR2, C0G6, SSO2, C0Y1, IMH1 and SEC31.

### g) Construction of an Expression Vector for Cloning of the Identified Secretion Helper Factors:

To generate a vector containing the GAP promoter and the his4 gene as selection marker, the AOX1 promoter of the vector pPIC9 (Invitrogen) was exchanged to the GAP promoter of pGAPZ B (Invitrogen) by restriction digest of both vectors with NotI and Mph1103I and subsequent ligation following a standard protocol. The newly constructed vector is referred to as pGAPHis.

h) Isolation of the Helper Factor Genes from Saccharomyces cerevisiae and Cloning into pGAPHis:

All the genes apart from Had were amplified directly from Saccharomyces cerevisiae genomic DNA by PCR with specific oligonucleotide primers depicted in Table 1. The P. pastoris Kozac sequence (ACG) was inserted directly before the start codon ATG. The non-template coded restrictions sites SacII (XhoI for the gene PDI1) and either PmII or Sfil (EcoR1 for the gene PDI1) were added by using the respective forward and backward primer (see Table 1). After restriction digest of the PCR fragments of correct length (checked by agarose gel separation) with SacII (XhoI for the gene PDI1) and either PmII or Sfil (EcoR1 for the gene PDI1) as shown in Table 1, these fragments were cloned into the pGAPHis vector (also digested with the respective restriction enzymes and treated with alkaline phosphatase). To construct the induced variant of the HAC1 gene of S. cerevisiae, the DNA fragment coding for the first 220 amino acids was combined with the fragment coding for the 18 amino acid exon of the induced Hac1p (Mori et al., 2000) in a two step PCR reaction, and the resulting fragment was ligated into pGAPHis.

All ligated plasmids were transformed into E. coli Top 10F′ (Invitrogen) and plated on Ampicillin containing LB-agar. Restriction enzyme analysis was performed to verify the correct identity of the respective plasmids.

TABLE 1
PCR primers for amplification of the
secretion helper factors from
Saccharomyces cerevisiae
(SEQ ID NO 1 to SEQ ID NO 31)

Forward primer BFR2 FORW PmII (SEQ ID NO 1),
54° C.:
5′-TAAACACGTGAGCATGGAAAAATCACTAGCGG-3′
Backward primer BFR2 BACK SacII
(SEQ ID NO 2), 56° C.:
5′-TACACCGCGGTCAACCAAAGATTTGGATATC-3′
Forward primer BMH2 FORW PmII
(SEQ ID NO 3), 56° C.
5′-TAACCACGTGAGCATGT:CCCAAACTCGTGAAG-3′
Backward primer BMH2 BACK SacII
(SEQ ID NO 4), 58° C.:
5′-TATGCCGCGGTTATTTGGTTGGTTCACCTTG-3′
Forward primer COG6 FORW PmII
(SEQ ID NO 5), 56° C.:
5′-TAAGCACGTGAGCATGGATTTCGTTGTAGACTAT-3′
Backward primer COG6 BACK SacII
(SEQ ID NO 6), 60° C.:
5′-TAAGCCGCGGTCAGTGATCAATACCTATCAAC-3′
Forward primer COY1 FORW PmII
(SEQ ID NO 7), 54° C.:
5′-TAGTCACGTGAGCATGGATACGTCAGTATATTC-3′
Backward primer COY1 BACK SacII
(SEQ ID NO 8), 58° C.:
5′-TACACCGCGGCTATCGATTTATGCCATGAAC-3′
Forward primer CUP5 FORW PmII
(SEQ ID NO 9), 54° C.:
5′-TATCCACGTGAGCATGACTGAATTGTGTCCTG-3′
Backward primer CUP5 BACK SacII
(SEQ ID NO 10), 54° C.:
5′-TACACCGCGGTTAACAGACAACATCTTGAG-3′
Forward primer ERO1 FORW Sfil
(SEQ ID NO 11), 62° C.:
5′-TATAGGCCCAGCCGGCCACGATGAGATTAAGAACCGCC
ATTG-3′
Backward primer ERO1 BACK SacII
(SEQ ID NO 12), 58° C.:
5′-TGTCCCGCGGTTATTGTATATCTAGCTTATAGG-3′
Forward primer IMH1 FORW Sfil
(SEQ ID NO 13), 54° C.:
5′-TATAGGCCCAGCCGGCCACGATGTTCAAACAGCTGTC
AC-3′
Backward primer IMH1 BACK SacII
(SEQ ID NO 14), 58° C.:
5′-TAGACCGCGGTTACTTCAGAGACATAACCAG-3′
Forward primer KIN2 FORW Sfil
(SEQ ID NO 15), 64° C.:
5′-TCAAGGCCCAGCCGGCCACGATGCCTAATCCGAATACAGC
AG-3′
Backward primer KIN2 BACK SacII
(SEQ ID NO 16), 66° C.:
5′-TCTGCCGCGGCTATAGGTTTAATTCTTTTAAAATATAC-3′
Forward primer KAR2 FORW PmII
(SEQ ID NO 17), 56° C.:
5′-TAAGCACGTGACGATGTTTTTCAACAGACTAAGC-3′
Backward primer KAR2 BACK SacII
(SEQ ID NO 18), 56° C.:
5′-TATGCCGCGGCTACAATTCGTCGTGTTCG-3′
Forward primer PDI1 FORW EcoRI
(SEQ ID NO 19), 58° C.:
5′-CGCCGAATTCACGATGAAGTTTTCTGCTGGTGC-3′
Backward primer PDI1 BACK Xhol
(SEQ ID NO 20), 58° C.:
5′-CCTCCTCGAGTTACAATTCATCGTGAATGGC-3′
Forward primer SEC31 FORW Sfil
(SEQ ID NO 21), 56° C.:
5′-TATAGGCCCAGCCGGCCACGATGGTCAAACTTGCTGAG
TT-3′
Backward primer SEC31 BACK SacII
(SEQ ID NO 22), 58° C.:
5′-TATGCCGCGGTTAATTCAAAGTCGCTTCAGC-3′
Forward primer SSA4 FORW PmII
(SEQ ID NO 23), 60° C.:
5′-TATGCACGTGACGATGTCAAAAGCTGTTGGTATTG-3′
Backward primer SSA4 BACK SacII
(SEQ ID NO 24), 58° C.:
5′-TATCCCGCGGCTAATCAACCTCTTCAACCG-3′
Forward primer SSO2 FORW PmII
(SEQ ID NO 25), 62° C.:
5′-TACACACGTGACGATGAGCAACGCTAATCCTTATG-3′
Backward primer SSO2 BACK SacII
(SEQ ID NO 26), 60° C.:
5′-TATGCCGCGGTTACTTTCTTGTTTCCACAACG-3′
Forward primer SSE1 FORW PmII
(SEQ ID NO 27), 60° C.:
5′-TAGACACGTGACGATGAGTACTCCATTTGGTTTAG-3′
Backward primer SSE1 BACK SacII
(SEQ ID NO 28), 60° C.:
5′-TATCCCGCGGTTAGTCCATGTCAACATCACC-3′
Forward primer HAC1 FORW Sfil
(SEQ ID NO 29), 58° C.:
5′-GCAAGGCCCAGCCGGCCACGATGGAAATGACTGATTTTGA
AC-3′
Backward primer HAC BACK1 (SEQ ID NO 30),
containing 3′-end of inactive
hac1pu (5′-splicing site), 58° C.:
5′-TGGTCATCGTAATCACGGC-3′
Backward primer HAC BACK2 SacII (SEQ ID NO
31), containing the sequence encoding the
last 18 aa of active hac1p, 58° C.:
5′-CCTCCCGCGGTCATGAAGTGATGAAGAAATCATTCAATTCA
AATGAATTCAAACCTGACTGCGCTTCTGGATTACGCCAATTGTC
AAG-3′

### Example 2

### Investigation of the Effect of the Secretion Helper Factors on Heterologous Protein Production of Recombinant 2F5 Fab in P. pastoris

The plasmid DNA from E. coli from Example 1 was used to transform P. pastoris strain SMD1168 already containing the expression cassettes for 2F5 Fab under control of the GAP promoter, which strain was pre-selected for a high Fab secretion level. The strain SMD1168 is a P. pastoris his4-defective strain (a pep4 mutant). Selection was based on zeocin resistance for the antibody genes, and histidin auxotrophy for the other genes.

a) Construction of the P. pastoris Strain SMD1168 Secreting the Fab Fragment of the Monoclonal Anti-HIV1 Antibody 2F5:

2F5 antibody fragment sequences for the Fab light and heavy chain were amplified by PCR from pRC/RSV containing the humanized IgG1 mAb as disclosed in Gasser et al., 2006. The restriction sites EcoRI and SacII were used for cloning.

In detail, for the generation of Fab, the entire light chain genes (vL and cL) and the vH and cH1 region of the heavy chain genes were amplified by PCR. The light chain fragment was ligated into a modified version of pGAPZαA, where the AvrII restriction site was changed into NdeI by site directed mutagenesis to allow subsequent linearization of the plasmids containing two cassettes. The heavy chain fragment was inserted into the original version of pGAPZαA, which contains the constitutive P. pastoris glycerol aldehyde phosphate dehydrogenase (GAP) promoter followed by the MFα leader sequence of S. cerevisiae (Invitrogen, Carlsbad, Calif., USA).

Plasmids combining the expression cassettes for both Fab chains on one vector were produced by double digestion of the light chain vector with Bgl II and BamHI, and subsequent insertion into the unique BamHI site of the vector pGAPZαA already containing a single copy of the expression cassette of the heavy chain fragment. Plasmids were then linearized with AvrII prior to electrotransformation into P. pastoris.

All constructed expression cassettes were checked by DNA sequencing with the GAP forw/AOX3′ back primers (Invitrogen).

b) Construction of P. pastoris Strains Co-Expressing 2F5 Fab and a Secretion Helper Factor:

Transformation of P. pastoris strains obtained in step a) was carried out with the plasmids of Example 1, which are linearized in the HIS4 locus. The plasmids were introduced into the cells by electrotransformation. The transformed cells were cultivated on RDB-agar (lacking histidine) for selection of His-prototrophic clones, which contain the expression cassettes for the secretion helper factors.

c) Culturing Transformed P. pastoris Strains in Shake Flask Cultures:

5 ml YP-medium (10 g/l yeast extract, 20 g/l peptone) containing 20 g/l glycerol were inoculated with a single colony of P. pastoris selected from the RDB plates and grown overnight at 28° C. Aliquots of these cultures corresponding to a final OD₆₀₀ of 0.1 were transferred to 10 ml of main culture medium (per liter: 10 g yeast extract, 10 g peptone, 100 mM potassium phosphate buffer pH 6.0, 13.4 g yeast nitrogen base with ammonium sulfate, 0.4 mg biotin) and incubated for 48 h at 28° C. at vigorous shaking in 100 ml Erlenmeyer flasks. To induce recombinant protein expression, cultures with the GAP promoter were supplemented with 10 g/l glucose. The same amounts of substrate were added repeatedly 4 times every 12 h, before cells were harvested by centrifugation at 2500×g for 5 min at room temperature and prepared for analysis (biomass determination by measuring optical density at 600 nm, ELISA for Fab quantification in the culture supernatant).

d) Evaluation of the Effect of Co-Overexpression of Single Folding Helper Factors by Quantification of 2F5 Fab:

To determine the amount of secreted recombinantly expressed 2F5 Fab, 96 well microtiter plates (MaxiSorb, Nunc, Denmark) were coated with anti-hIgG (Fab specific) overnight at RT (Sigma I-5260;1:1000 in PBS, pH 7.4), before serially diluted supernatants of P. pastoris cultures secreting 2F5 Fab from step c) (starting with a 1:200 dilution in PBS/Tween20 (0.1%)+1% BSA) were applied and incubated for 2 h at RT. A human Fab of normal IgG (Rockland) was used as a standard protein at a starting concentration of 200 ng/ml. After each incubation step the plates were washed four times with PBS containing 0.1% Tween 20 adjusted to pH 7.4. 100 μl of anti-kappa light chain—AP conjugate as secondary antibody (1:1000 in PBS/Tween+1% BSA) were added to each well, and incubated for 1 h at RT. After washing, the plates were stained with pNPP (1 mg/ml p-nitrophenyl phosphate in coating buffer, 0.1 N Na₂CO₃/NaHCO₃; pH 9.6) and read at 405 nm (reference wavelength 620 nm).

Of each of the 15 different secretion helper factor constructs, 16 individual clones were cultivated in shake flask cultures as described in step c) and compared to 16 individual clones of the control strain, that was transformed with the pGAPHis vector lacking a gene. The 2F5 Fab productivity (μg Fab/biomass) was determined for all the analyzed cultures (first screening round). The 6 best clones of each of the constructs were then re-analyzed using the same system in a second screening round (for results see Table 2).

Table 2 shows the mean relative productivity of the 6 best clones of each tested secretion helper factor construct including the control construct (empty pGAPHis vector). The table shows the mean improvement factor of 2F5 Fab secretion of two screening rounds obtained by co-overexpression of the secretion helper factors relative to the control cultures. The secretion helper factors which are known in the art improving the secretion of heterologous proteins when co-overexpressed (PDI1, KAR2, HAC1, ERO1 and SSO2) are included in Table 2 for comparative reasons.

TABLE 2
Mean relative productivity of the tested secretion helper factors

| Secretion helper | Mean |
|---|---|
| Factor | Improvement |
| PDI | 1.7 |
| CUP5 | 1.7 |
| SSA4 | 1.6 |
| BMH2 | 1.5 |
| KIN2 | 1.5 |
| KAR2 | 1.5 |
| HAC1 | 1.5 |
| ERO1 | 1.4 |
| SSE1 | 1.4 |
| BFR2 | 1.4 |
| COG6 | 1.2 |
| SSO2 | 1.2 |
| COY1 | 1.2 |
| IMH1 | 1.1 |
| control | 1.0 |

As can be seen from Table 2, that the secretion of the heterologous protein, i.e. the secretion of 2F5 Fab was increased for most of the analyzed secretion helper factors, in a range between 1.2 and 1.7-fold. Apart from the secretion helper factors already known in the art having a positive effect on the secretion of a heterologous protein co-overexpression of the secretion helper factors CUP5, SSA4, BMH2, KIN2, SSE1 and BFR2 showed a highly significant increase in the amount of secreted heterologous protein and co-overexpression of C0G6, C0Y1 and IMH1 showed a significant increase in the amount of secreted heterologous protein.

Sequence information for the secretion helper factors PDI1, KAR2, HAC1, ERO1 and SSO2 is disclosed in the prior art.

The nucleotide sequences of the secretion helper factors which are not yet known in the art improving the secretion of heterologous proteins when co-overexpressed are shown in Table 3 below.

TABLE 3
Nucleotide sequences of the isolated secretion
helper factors (SEQ ID NO 32 to SEQ ID NO 41)

S. cerevisiae BMH2 (SEQ ID NO 32)
ATGTCCCAAACTCGTGAAGATTCTGTTTACCTAGCTAAATTAGCTGAAC
AAGCCGAACGTTATGAAGAAATGGTCGAAAACATGAAGGCCGTTGCTTC
ATCAGGTCAAGAGTTATCTGTCGAAGAACGGAATCTATTGTCGGTTGCT
TACAAGAACGTCATCGGTGCTCGCCGTGCTTCATGGAGAATAGTTTCTT
CGATCGAACAAAAAGAAGAATCAAAGGAGAAATCTGAACATCAAGTTGA
ATTAATCCGTTCTTACCGTTCTAAAATTGAAACTGAATTGACCAAAATC
TCTGACGACATTTTATCTGTGTTAGATTCTCATTTAATCCCTTCTGCTA
CTACTGGTGAGTCTAAAGTATTTTACTATAAGATGAAGGGTGACTACCA
CCGTTATTTAGCTGAATTTTCCAGCGGAGATGCAAGAGAAAAGGCAACC
AACTCCTCTTTGGAGGCTTATAAAACCGCTTCCGAAATCGCCACAACTG
AATTGCCTCCAACTCACCCAATTCGTTTAGGTCTAGCTTTGAATTTCTC
CGTCTTCTATTACGAAATTCAAAACTCTCCTGATAAGGCTTGCCACTTG
GCCAAACAAGCCTTTGATGATGCTATTGCTGAGTTAGATACTTTATCTG
AAGAATCATACAAGGATAGCACTTTGATCATGCAATTATTAAGGGACAA
CTTGACCTTATGGACCTCTGATATTTCTGAATCTGGTCAAGAAGATCAA
CAACAACAACAACAACAGCAACAGCAACAGCAACAACAGCAACAACAAG
CTCCAGCTGAACAAACTCAAGGTGAACCAACCAAATAA
S. cerevisiae BFR2 (SEQ ID NO 33)
ATGGAAAAATCACTAGCGGATCAAATTTCCGATATCGCCATTAAACCGG
TCAATAAAGACTTCGATATTGAAGATGAGGAAAATGCATCTTTATTTCA
ACACAATGAAAAAAATGGAGAAAGTGATTTAAGCGACTATGGAAATAGC
AACACAGAAGAAACCAAGAAGGCGCACTATTTGGAGGTGGAAAAGTCTA
AGTTAAGAGCAGAAAAAGGTTTAGAACTAAACGATCCAAAATATACAGG
TGTTAAAGGTTCAAGACAAGCATTATATGAAGAAGTTTCCGAGAATGAG
GACGAAGAAGAAGAAGAAGAAGAGGAAGAAGAAAAAGAGGAAGATGCTC
TTTCATTCAGGACAGATTCTGAAGATGAAGAAGTAGAGATTGATGAAGA
AGAATCAGACGCGGACGGCGGTGAAACGGAGGAGGCTCAACAGAAAAGG
CATGCACTATCGAAACTAATTCAACAAGAGACTAAACAAGCTATTAACA
AACTGTCTCAATCAGTTCAAAGAGATGCTTCGAAGGGTTATTCCATTTT
ACAACAGACAAAATTATTTGACAACATCATTGATTTGAGAATAAAACTA
CAAAAAGCTGTAATTGCAGCAAATAAGCTCCCATTAACTACAGAGTCCT
GGGAAGAGGCTAAAATGGATGATTCAGAGGAAACAAAGCGTTTGCTGAA
GGAAAACGAAAAACTGTTCAATAATTTATTCAATCGGTTGATAAATTTC
AGAATAAAATTCCAACTTGGCGATCATATCACTCAAAATGAAGAGGTGG
CGAAGCATAAATTGTCCAAAAAAAGATCTCTCAAAGAGCTTTACCAAGA
AACTAATAGCTTAGACTCAGAACTAAAAGAGTACAGGACTGCCGTATTA
AACAAGTGGTCTACCAAAGTTTCTTCTGCATCAGGTAACGCTGCTTTAT
CATCTAACAAATTCAAAGCTATCAACTTACCTGCAGATGTACAAGTCGA
AAACCAATTATCCGATATGTCCCGTTTGATGAAAAGAACAAAGTTGAAC
AGGAGAAACATAACGCCTTTGTATTTCCAAAAAGACTGTGCTAATGGCA
GGCTACCAGAATTGATTTCTCCCGTTGTCAAAGATAGTGTTGATGACAA
TGAGAATTCGGATGATGGGCTTGATATCCCGAAAAACTATGACCCAAGA
AGAAAGGATAACAATGCCATTGACATTACCGAAAACCCATATGTTTTTG
ATGACGAAGATTTTTACCGTGTTTTACTAAACGATTTAATTGACAAAAA
GATTTCCAACGCTCACAATTCTGAAAGTGCAGCAATTACAATCACCTCA
ACTAATGCTCGTTCGAACAACAAGCTAAAGAAGAATATCGATACTAAGG
CTTCCAAGGGTAGGAAATTGAACTACTCAGTTCAAGATCCAATTGCGAA
TTATGAAGCCCCCATCACATCCGGATACAAATGGTCAGACGACCAAATC
GATGAATTCTTTGCGGGATTGTTAGGTCAACGAGTGAACTTTAATGAAA
ATGAGGATGAGGAACAACATGCCAGAATAGAAAATGACGAAGAATTAGA
GGCTGTTAAAAACGATGATATCCAAATCTTTGGTTGA
S. cerevisiae COG6 (SEQ ID NO 34)
ATGGATTTCGTTGTAGACTATCAGACCTACGCAATGGCGGATACTGCCA
CGCCAGAATTACCAGAACCTGAGCCAAGACTAAACTTAACCTCAGATGC
ACAGTCACAGCCCACCGGTAAACTAGATCTACAGTTTAAGTTGCCCGAC
CTTCAACGTTATTCCAATAATAATGCAACTTTGCCAGTAGATAATGATG
GTGCTGGTTCGAAAGACCTACATAAGAAAATGACACATTACGCAATGTC
TTCCATTGATAAAATACAGCTTTCAAATCCAAGCAAACAATTAGGGCAA
AATTCCCAGGATGAAAAACTATCGCAGCAAGAATCTCAAAATTTCACGA
ATTACGAGCCAAAAAACCTTGATTTATCAAAATTAGTATCCCCGTCAAG
TGGTTCCAACAAAAATACCACAAATTTGGTTCTTTCGAATAAACTATCC
AAGATATTGAACAATTACACATTGATTAACTATCAGGCCACAGTCCAAC
TAAGAAAATCCCTAAAGGTTCTAGAAGAGAATAAAGAGAGATTGTCCCT
TGATGAACAAAAGCTCATGAATCCTGAATATGTAGGTACTTTGGCAAGA
AGAGCATTGAGGACTGATTTGGAATCTCAACTGCTAAAGGAACATATTA
CGGTACTTGAGGAATTCAAACCTATCATTAGAAGGATTAAACGATTATC
TTCTTCCGTCGAAAAAATACAAAGAACGAGCGAAAAATTACTAAGTAAT
GAGACAAATGAGGTTCCAACAAATAACGTGGTACTTCAGGAAATAGATC
AATACCGTTTAAAGGCAGAGCAGTTGAAGCTGAAAAAAAAAATACTGTT
ATCTATAAGGGATAGGTTTACTTTGAATCAGGTAGAGGACGATGTAATC
ACCAATGGTACTATAGACAACATCTTTTTCGAGGTAGTAAAGAAAGTAA
TCAATATTAAAGATGAATCAAGTTTCTTGCTGACGCTTCCTAATTTGAA
TGCTGGAAATGCTTTGATAATGGGAGTTAATGAAATTTTAGAAAAGACA
AACAAAAAAATCTTCAATTATTTGATCGATTTTTTATATAGTTTTGAAT
CCTCTTCAAATTTATTAAATGACCATGGTACTACTGAACAAGAAAGCTT
AAACATTTTTCGGAAGAGTCTGGTCTTCCTGTCAAGTGATCTAGAATTA
TTTAATGAGTTGTTGAAAAGAGTGACCACACTGAGATCCAAGAGTATTC
TGGATGAGTTTTTGTCTCAATTCGATATGAATTCAACTACCTCTAAACC
CATCATATTATCGGCACACGATCCAATTAGGTATATTGGTGACGTACTA
GCGTCCGTTCATTCCATCATCGCAAATGAAGCTGATTTCGTGAAGTCAC
TATTTGACTTTCAGGATGAAGACTTAAAAGATACCCCAATTTCTATACT
TCAACAAAACAAGACATTCTTGAAAGGCATCGACAACAAATTGCTGAAC
GATATCATCCAGTCGCTATCCAATTCGTGTCGTATTCGTATCGAGCAAA
TCGTGAGGTTTGAAGAAAATCCGATCATCAATTTCGAGATTGTGAGGCT
GCTGAAACTTTACAGAGTTATGTTCGAGAGAAAGGGAATTCAGGACGAT
AGTTCTATTATTAACAATTTAAAGTCGTTGGAAGACATTTCCAAAAACA
GAATTATTGGATACTATGAAGACTATATGAAGCAAACAGTCATGGCGGA
AACAAAAAATTCTTCAGATGATTTACTGCCACCAGAGTGGCTATCAGAG
TATATGAATAAATTGGTAGAGTTATTTGAAATTTATGAAAAGACACATG
CTGCCGAAGATGAGGAATCAGAAGATAATAAATTGCTCTCATCTAAGAA
TTTACAAACAATTGTAGAACAACCAATAAAAGATGTTCTGTTAAAACAA
TTGCAAACATCTTTTCCTTTGGCGAAAAAAAATGAAAAAGAAAAGGCAT
CATTGCTAACTATAGAGATAAACTGTTTCGATTTAATTAAATCTAGACT
TCAACCTTTTGAGGGATTGTTTGCACAAGATGATGACAGCCGGAAAATC
ACCATCTGGGTTTGTGATAAACTGAAGGAATATACTAAGCAAATGCTAA
CTTTACAAATAAAATTCCTATTTGAGAATACAGGTTTAGACCTTTACAG
CAATTTGGTCAATATGATTTTTCCTGTGGACTCAGTAAAGGATGAATTG
GATTATGATATGTACTTAGCCCTGAGGGATAATTCATTGATGGAATTAG
ACATGGTCAGAAAAAATGTGCATGATAAGTTGAACTATTATCTACCTCA
GGCGTTAACAGATGTTCAAGGTAATTTACTATTTAAATTAACGTCACCA
ATGATAGCTGATGAAATATGCGATGAATGTTTCAAGAAGTTGTCGCTAT
TTTATAATATCTTCAGGAAACTGTTGATTCATTTGTATCCGAACAAGAA
GGATCAGGTATTCGAAATTTTAAATTTTTCCACTGATGAATTTGACATG
TTGATAGGTATTGATCACTGA
S. cerevisiae COY1 (SEQ ID NO 35)
ATGGATACGTCAGTATATTCTCATGCATTGGATATTTGGGCCAAGGCAG
ATTTAACGAATCTTCAAAGAGAATTGGATGCTGATGTTATAGAGATTAA
GGATAAAGAAACCCTGTCCTTGAATTCAAGAAAGTCATTAGCCACTGAG
ACTAAAAAATTTAAAAAACTCGAACCTGAGGAAAAATTGAACAATGTGA
ATAAAATAATTAAGCAGTACCAACGTGAAATTGATAATTTGACACAGAG
ATCAAAATTCTCTGAAAAGGTTCTTTTTGACGTATACGAAAAGCTTTCA
GAGGCTCCTGATCCACAGCCGCTACTACAAAGTTCGTTGGAAAAATTGG
GCAAAATTGATGACTCGAAGGAACTTAAGGAAAAAATAAGCTACCTAGA
AGATAAGCTAGCCAAATATGCAGATTATGAGACTTTGAAATCAAGGTTA
CTGGACCTAGAGCAAAGCTCTGCAAAAACATTGGCAAAAAGACTGACTG
CGAAAACTCAAGAAATCAATTCTACCTGGGAGGAAAAAGGAAGAAATTG
GAAAGAGAGAGAAGCAGATCTATTGAAACAATTAACAAATGTACAGGAG
CAAAACAAGGCACTAGAGGCCAAAATATCTAAAAATATAGATATAGAAG
GTAATGGAAACGAAGATGGTGACCAAGAAAACAATCAAAAAGAAGTATC
TACAAGGATTGCTGAATATAATCTAGTAACACAGGAGTTGGAAACTACG
CAGGCTAGAATATATCAGTTAGAGAAAAGAAATGAGGAACTAAGTGGTG
CTCTTGCAAAGGCAACTAGTGAAGCAGAAAAAGAAACTGAGTTACATGC
AAAGGAACTAAAACTTAACCAGCTGGAAAGCGAAAATGCATTGTTGAGT
GCATCCTATGAGCAGGAACGGAAATCAACATCACATGCAATAAATGAGT
TAAAAGAACAATTAAATAGCGTTGTGGCGGAATCGGAATCTTACAAGTC
GGAGCTAGAAACTGTTAGAAGAAAACTAAACAATTATTCTGATTACAAT
AAGATAAAAGAAGAACTTTCTGCATTGAAAAAAATTGAGTTTGGGGTAA
ACGAAGATGATTCTGATAATGACATTCGCTCTGAAGACAAGAATGATAA
TACTTTCGAAAGTTCCTTACTATCTGCAAATAAGAAGCTCCAGGCTACT
TTGGCGGAATACCGCTCAAAAAGTACGGCTCAAGAGGAAGAACGAAACG
AATTGAAAAAATCTGTGGACCAATTGAAGCAGCAAATAGCTACTCTCAA
AGAAGCAAATGAAAAATTAGAGACGGACCTAGAAAAAGTAGAGAACGTC
AGTCCTCACTTCAACGAGACTGCAAGTATGATGTCTGGTGTAACAAGAC
AAATGAACAATCGTACGTCCCATAAAATGTCCCCAACGAGTTCTATTAT
TGGTATTCCAGAAGATGGGGAACTTTCTGGAAACCAATCAACCATTTTA
CCAATAGTTACTAAACAAAGAGACAGATTTCGTTCGAGAAATATGGATC
TGGAAAAGCAACTAAGACAAGGAAACTCAGAAAAGGGTAAGCTTAAACT
AGAAATTTCGAAGCTAAAAGGCGACAATACGAAGCTTTATGAACGGATT
AGGTATCTGCAATCCTATAATAATAACAACGCTCCCGTTAATCAAAGTA
CAGAGCGTATTGACGTGGAATCCCAATACTCAAGGGTGTATGATGAATC
GTTGCATCCAATGGCAAATTTTAGACAGAACGAATTAAACCACTACAAA
AACAAGAAATTATCAGCTTTAGAGAAGTTATTTTCCAGTTTTGCAAAAG
TCATTTTACAAAATAAAATGACAAGGATGGTATTCCTCTTTTACTGTAT
CGGTTTACACGGACTCGTATTCATGATGAGCATGTATGTGATTAATATT
AGCGGCTACATGACACCTGAGGTTGGTATAGTACAATCGGCAAAGTCTT
CTTCAAATCTCAACGGAGGACTTGGGGGAGCAGAAAAAGTAGCTGCAGG
CGTTGGTTCAGTTCATGGCATAAATCGATA
S. cerevisiae CUP5 (SEQ ID NO 36)
ATGACTGAATTGTGTCCTGTCTACGCCCCTTTCTTTGGTGCCATTGGTT
GTGCCTCTGCAATTATCTTCACCTCATTAGGTGCTGCTTACGGTACTGC
TAAGTCTGGTGTTGGTATCTGTGCCACTTGTGTGTTGAGACCAGACCTA
TTATTCAAGAACATTGTTCCTGTTATTATGGCTGGTATCATTGCCATTT
ACGGTTTAGTTGTTTCCGTTTTGGTTTGTTATTCGTTGGGTCAAAAGCA
AGCTTTGTACACCGGTTTCATCCAATTGGGTGCCGGTCTATCAGTCGGT
TTGAGTGGTCTAGCTGCTGGTTTCGCTATTGGTATTGTCGGTGATGCAG
GTGTTAGAGGTTCCTCTCAACAACCAAGATTATTCGTCGGTATGATTTT
GATTTTGATTTTTGCTGAAGTTTTGGGTCTATACGGTTTGATTGTTGCT
TTGTTGTTGAACTCCAGGGCTACTCAAGATGTTGTCTGTTAA
S. cerevisiae IMH1 (SEQ ID NO 37)
ATGTTCAAACAGCTGTCACAAATTGGTAAGAATCTTACCGATGAATTAG
CGAAGGGCTTAGCCGATGATATGAGCCCTACCCCGTCAGAACAACAAAT
CGAAGATGATAAGAGTGGCTTGCCAAAAGAAATACAAGCTAAATTAAGA
AAATTTGAGAAATATGAACAAAAATACCCTTTGCTACTCTCCGCATACA
AAAATGAAAAATTAAAGTCAGAGAAGTTAGAGGCTGTTGAAAAGATTTT
AGCGGAAAATACACCCATATCTAATATTGACGACGCAGTGGATACGTTG
CCAGCTTTTTTCCAGGATTTAAACAACAAAAATAACCTATTGAATGATG
AGATCAAGAGATTAACTAAGCAGAACTCGGAAATTCCAGAAAGCGCCTC
TAGTGAAACTCTGAAGGATAAAGAAGAGGAATTTTTGAAAAAAGAGCAA
AATTATAAAAATGACATAGACGATCTAAAAAAAAAAATGGAAGCTTTAA
ACATAGAATTGGATACTGTACAAAAAGAAAAAAATGATACTGTTTCAGG
TTTGAGAGAAAAAATAGTTGCACTGGAAAATATACTAAAGGAAGAAAGG
GAGGCCAAAAAACAGAAAGAAGAAGTATCTATATCCGAACTGAAGGAAG
AATTGGCTATAAAGAACCATTCTCTCGAGGACAGTCGAATGAAGATAAC
CGAATTGGAGCAAAATTTGTCTTCGAAAAGTACTATAATGGAGGAAAAG
TCCTCAGAGTTGGCAGAACTAAATATTACTTTAAAAGAGAAAGAGCGCA
AGCTGAGTGAATTGGAAAAAAAAATGAAGGAGTTACCGAAGGCGATATC
TCATCAAAATGTAGGAAACAATAACAGAAGGAAAAAGAATAGAAACAAG
GGAAAGAAAAATAAGGGAGGCATAACTACGGGTGATATCAGTGAAGAGG
AAACGGTCGATAACTCAATCAATACTGAAGAATATGATAAGCTTAAAGA
AAATTTGCAAGAATTACAAGAAAAATATAAAGATTGTGAAGATTGGAAG
CAAAAGTATGAAGATATAGAAGCAGAACTAAAAGATGCTAAAGAATTGG
AAAACTCACAGCTCGAAAAATCAGCAAAGGAGCTGGAAACCCTTAACAC
CGAGTTGATCGATACCAAGAAGTCATTGAAAGAAAAAAATTCGGAGCTA
GAGGAGGTGAGAGATATGCTGAGGACTGTAGGCAATGAGCTTGTGGACG
CAAAAGATGAGATTAAAGAGTCTTCGAGTAAACAAAATGAAGAAGTGAA
AACCGTTAAGCTGGAGCTCGATGATTTACGCCATAAAAATGCAACGATG
ATCGAGGCCTACGAAGCTAAAAATACTGAGTTGAGAAGTAAGATAGAGT
TATTGAGCAAGAAAGTAGAGCATCTGAAGAATTTATGTACAGAAAAGGA
GAAAGAGCAGACTACATCGCAGAACAAGGTAGCCAAATTAAATGAGGAG
ATATCTCAACTTACCTACGAAAAATCAAACATAACAAAGGAGCTTACTT
CTTTAAGAACCTCTTATAAACAAAAGGAGAAAACTGTGAGTTACTTGGA
GGAACAAGTTAAACAATTTAGTGAGCAAAAGGACGTGGCTGAAAAATCC
ACAGAACAGCTGAGAAAAGATCATGCTAAAATTTCTAACAGATTAGACT
TATTAAAAAAGGAAAATGAGACACTGCATAATGATATCGCAAAGAATTC
TAATTCCTACGAGGAGTATTTGAAAGAAAATGGTAAATTATCGGAAAGA
TTGAATATTTTGCAAGAAAAATACAATACCTTGCAAAATGTAAAAAGTA
ATTCGAATGAACACATAGATTCTATCAAAAGACAATGTGAGGAACTAAA
TGTCAAGTTGAAGGAATCTACAAAAAAAATTTTATCTTTAGAAGATGAA
CTAAATGAATATGCTAATATTGTTCAAGACAAAACCAGAGAAGCTAACA
CATTGAGAAGGTTAGTTTCGGACAGTCAGACAGATGATTCGAGCAAACA
AAAAGAGTTGGAGAATAAATTGGCCTATTTAACGGATGAAAAGAATAAA
TTGGAAGCAGAATTAGACTTACAAACATCCAGAAAGGCCACTGAATTAC
AAGAGTGGAAGCATACAGTAACTGAGCTGAAATCGGAAATACACGCTTT
AAAGCTTCGTGAAGAGGGACTAAAATCAGAGGTTGACGCATTGAAACAT
GTTAACAATGACATCAAAAGGAAGACTCAAGCCACTTCAGATGATTCCG
ATCAGTTGGAACAGATCACATCTAATTTAAAACTCTCATTGTCTAAGGC
TGATGAAAAGAATTTTGAGCTACAGTCTGCCAATGAGAAACTTCTGAAT
TTAAATAACGAACTTAACAAGAAATTTGATCGATTACTAAAAAATTATC
GTTCATTGTCCTCTCAATTGAATGCTTTAAAGGAAAGACAATACAGTGA
CAAGTCAGGAAGAGTTAGTAGGTCTGGTTCTATCGGTACTCTAGCTAAC
GCGAATATTGATTCCTCACCAGCGAATAACTCTAATCCAACTAAATTAG
AGAAGATACGATCATCAAGTTCATTGGAGTTAGACTCTGAGAAAAATGA
AAAAATTGCATATATAAAAAATGTTTTGTTGGGATTTTTGGAGCACAAG
GAACAACGGAACCAATTACTTCCTGTAATTTCTATGTTGTTACAACTGG
ACAGTACTGATGAAAAAAGACTGGTTATGTCTCTGAAGTAA
S. cerevisiae KIN2 (SEQ ID NO 38)
ATGCCTAATCCGAATACAGCAGATTACTTGGTGAATCCAAATTTCAGGA
CCAGTAAGGGCGGATCTTTATCGCCGACGCCAGAAGCTTTCAACGACAC
GCGAGTTGCTGCACCAGCCACTCTTCGCATGATGGGCAAGCAATCTGGA
CCAAGAAATGACCAGCAACAAGCACCACTGATGCCTCCTGCAGATATCA
AACAGGGCAAGGAACAGGCAGCTCAGAGACAAAATGATGCATCGAGGCC
TAATGGCGCCGTGGAATTAAGGCAATTTCATAGAAGATCTTTGGGAGAT
TGGGAGTTCCTTGAAACGGTTGGCGCAGGCTCTATGGGTAAAGTTAAAT
TGGTCAAGCATCGTCAAACAAAGGAAATTTGTGTAATAAAGATTGTTAA
TAGGGCTTCCAAGGCTTATCTCCATAAACAGCACTCTTTACCTTCCCCA
AAGAATGAGAGTGAGATATTAGAAAGACAAAAGCGGTTAGAAAAAGAAA
TTGCGAGGGATAAAAGGACTGTTAGGGAAGCCTCTTTGGGCCAAATCCT
TTACCATCCTCATATCTGTCGTTTATTTGAAATGTGCACTATGTCAAAC
CATTTTTATATGCTTTTTGAATACGTTTCCGGTGGACAGCTGTTAGATT
ATATTATTCAGCATGGCTCATTAAAGGAACACCATGCGAGGAAATTTGC
ACAGAGGTATAGCTAGTGCGCTGCAATACTTACTGCCAATAATATTGTT
CATCGAGATCTGAAAATTGAGAATATAATGATATCTAGTTCAGGTGAAA
TTAAGATCATTGATTTTGGTCTTTCCAACATTTTTGATTATAGGAAACA
ATTACATACGTTTTGTGGTTCCTTGTACTTTGCAGCACCAGAACTATTA
TAAAGCGCAGCCATACACAGGACCTGAGGTAGATATTTGGTCGTTGGTA
TTGTTCTTTATGTCTTGGTCTGCGGTAAAGTACCATTTGATGATGAGAA
CTCAAGCATTTTACATGAAAAAATAAAAAAAGGTAAAGTAGACTATCCT
TCACACTTATCCATTGAAGTTATATCTTTATTAACCAGGATGATTGTTG
TCGACCCATTAAGAAGAGCAACATTAAAGAATGTCGTTGAGCATCCATG
GATGAACAGAGGATACGATTTTAAGGCTCCATCATATGTTCCTAATCGT
GTTCCATTAACCCCTGAAATGATAGATAGCCAAGTTCTGAAGGAAATGT
ATCGCCTAGAATTTATTGACGATATTGAAGATACAAGAAGATCATTGAT
CCGATTAGTAACTGAAAAGGAATACATCCAACTTTCCCAAGAATACTGG
GACAAATTATCCAACGCCAAGGGGTTGAGTTCAAGTTTAAATAATAACT
ACCTAAATTCAACGGCACAACAAACCTTAATACAAAATCATATTACAAG
TAATCCATCGCAAAGTGGTTATAATGAACCAGATAGTAATTTTGAAGAT
CCTACTTTAGCATATCATCCATTACTATCAATATATCACTTGGTTTCAG
AAATGGTTGCACGGAAATTAGCGAAGTTGCAAAGAAGGCAAGCATTGGC
CCTGCAAGCGCAAGCTCAGCAAAGGCAACAACAGCAACAAGTAGCACTT
GGCACTAAGGTCGCCTTAAATAATAACTCCCCGGATATTATGACCAAAA
TGAGGAGCCCTCAGAAAGAAGTAGTACCTAATCCTGGTATTTTTCAAGT
GCCGGCAATTGGAACATCGGGAACCTCAAACAACACTAATACCTCAAAC
AAACCTCCACTGCATGTAATGGTTCCTCCTAAACTAACAATACCGGAAC
AAGCGCATACTTCTCCAACATCTAGGAAGAGTTCCGACATTCATACGGA
ATTAAATGGTGTTTTGAAATCAACACCAGTCCCCGTGTCTGGCGAATAT
CAGCAACGTTCTGCTTCACCCGTAGTAGGTGAACATCAGGAAAAGAATA
CAATAGGCGGCATATTCAGAAGAATATCACAAAGTGGACAATCTCAGCA
TCCCACACGGCAACAGGAACCTCTTCCAGAAAGAGAACCTCCAACATAT
ATGTCAAAATCAAATGAAATTTCCATCAAAGTACCGAAAAGCCATAGTC
GTACTATATCAGATTATATTCCTAGCGCTAGAAGATATCCATCTTACGT
GCCAAATTCTGTTGATGTAAAACAGAAACCCGCTAAAAACACTACCATA
GCACCTCCTATAAGGTCAGTATCACAAAAGCAAAACAGTGATCTTCCAG
CTTTACCTCAGAACGCCGAACTAATTGTTCAAAAACAACGGCAAAAACT
ATTACAGGAAAATCTCGACAAATTACAAATTAATGATAATGATAACAAC
AATGTGAACGCTGTAGTCGATGGTATCAATAATGATAATAGTGACCATT
ATCTCTCCGTTCCGAAGGGTCGTAAGTTACATCCTAGTGCAAGGGCTAA
ATCGGTGGGGCATGCTCGTCGTGAATCTTTGAAATTTACTAGGCCGCCT
ATACCAGCAGCCCTTCCGCCATCAGATATGACAAACGATAACGGCTTTT
TGGGAGAGGCAAACAAGGAGAGATACAATCCTGTTAGCAGTAACTTTTC
GACCGTTCCTGAAGATTCTACCACATACAGTAACGATACTAACAATAGA
CTGACTTCGGTGTATTCTCAGGAGCTTACTGAGAAGCAAATTTTGGAGG
AAGCTTCAAAGGCACCCCCCGGGTCTATGCCATCAATTGATTATCCAAA
GTCAATGTTTTTGAAGGGTTTTTTCTCTGTACAAACAACCTCCTCTAAA
CCATTGCCTATTGTTCGTCACAATATCATATCTGTTTTAACAAGAATGA
ATATTGATTTCAAAGAAGTGAAAGGCGGGTTCATATGTGTCCAACAAAG
GCCATCTATTGAGACTGCAGCTGTCCCTGTTATAACCACTACTGGCGTG
GGTTTGGATTCCGGAAAGGCGATGGATCTGCAAAATAGTTTAGACAGTC
AATTATCATCCAGTTACCATAGTACAGCGTCCTCAGCATCAAGAAATAG
TTCGATAAAACGCCAAGGTTCTTATAAGAGGGGCCAGAATAATATACCA
CTAACACCTTTAGCGACCAATACACATCAAAGAAATTCATCTATCCCAA
TGTCTCCAAACTACGGAAACCAAAGTAATGGTACATCAGGGGAACTATC
TTCCATGTCATTAGATTATGTTCAACAACAGGATGATATTTTAACAACA
TCAAGAGCCCAAAATATAAATAACGTAAATGGTCAAACAGAGCAAACCA
ATACTTCTGGTATAAAAGAAAGGCCTCCTATTAAATTTGAGATTCACAT
TGTAAAGGTTCGTATCGTCGGCCTAGCAGGTGTACATTTCAAAAAGGTT
TCTGGTAATACGTGGCTATATAAAGAATTGGCATCGTATATTTTAAAAG
AATTAAACCTATAG
S. cerevisiae SEC31 (SEQ ID NO 39)
ATGGTCAAACTTGCTGAGTTTTCTCGAACAGCCACGTTTGCGTGGTCAC
ATGATAAAATTCCATTATTGGTCTCTGGTACCGTATCTGGTACGGTGGA
TGCTAATTTCTCCACTGATTCATCTCTAGAATTGTGGTCATTGTTGGCT
GCTGATTCGGAGAAGCCTATTGCTTCCTTGCAAGTGGATTCCAAATTCA
ATGATTTGGATTGGTCTCATAATAACAAGATTATTGCTGGTGCTCTGGA
TAACGGTAGTTTGGAATTGTACTCCACCAATGAAGCAAACAACGCTATC
AACTCCATGGCCAGATTTAGCAACCATTCTTCCTCTGTGAAGACGGTAA
AGTTTAACGCAAAGCAAGACAACGTTCTTGCTTCGGGTGGTAACAACGG
TGAAATTTTTATTTGGGACATGAATAAATGCACTGAATCGCCCTCCAAT
TATACTCCATTGACACCGGGTCAATCGATGTCGTCCGTTGACGAGGTCA
TTTCCCTAGCATGGAACCAATCTTTGGCCCATGTTTTTGCATCTGCCGG
GTCGTCTAATTTCGCATCTATTTGGGATTTGAAGGCTAAGAAGGAAGTC
ATTCATCTAAGTTACACTTCACCTAATTCAGGTATCAAGCAACAGCTGT
CCGTTGTTGAATGGCACCCAAAAAACTCCACAAGAGTGGCAACGGCTAC
TGGTAGCGATAATGATCCATCTATCCTGATCTGGGATTTAAGAAACGCC
AACACACCATTGCAGACTTTAAATCAAGGCCATCAAAAGGGTATTTTGT
CATTAGATTGGTGTCATCAGGACGAACATCTATTATTGTCCAGTGGTAG
AGATAATACCGTTCTTCTATGGAACCCTGAGTCAGCCGAACAACTGTCC
CAATTCCCAGCTCGTGGAAACTGGTGTTTTAAGACCAAATTTGCACCAG
AGGCTCCAGACCTATTTGCTTGTGCCTCCTTTGATAACAAAATTGAGGT
ACAGACTTTGCAAAATCTCACAAACACTTTGGATGAGCAAGAAACCGAA
ACTAAGCAGCAAGAATCTGAAACAGATTTTTGGAATAATGTTTCCCGAG
AGGAATCAAAAGAGAAGCCATCTGTTTTCCATTTACAAGCCCCAACTTG
GTATGGGGAACCATCTCCCGCAGCTCATTGGGCTTTCGGTGGTAAATTG
GTTCAAATTACTCCAGATGGTAAAGGTGTATCTATAACAAACCCAAAAA
TTTCAGGCTTAGAATCAAACACTACTTTGAGTGAAGCGTTGAAAACTAA
GGATTTCAAACCATTAATAAATCAAAGACTGGTCAAAGTTATTGATGAC
GTTAATGAAGAAGATTGGAATTTATTGGAAAAGTTATCAATGGACGGTA
CTGAGGAGTTCTTGAAAGAGGCTCTTGCATTCGACAACGATGAATCAGA
TGCACAAGACGATGCCAACAATGAGAAAGAAGACGATGGGGAAGAATTC
TTTCAACAAATTGAAACCAATTTCCAACCCGAGGGCGATTTCTCCTTGT
CTGGTAATATCGAACAAACTATTTCCAAGAACTTGGTTTCTGGCAACAT
TAAGAGCGCTGTGAAAAATTCTCTAGAGAATGACTTACTAATGGAGGCC
ATGGTGATCGCATTAGATTCAAATAACGAAAGATTAAAGGAAAGTGTCA
AGAATGCCTATTTTGCGAAGTATGGATCTAAATCATCGCTCTCGAGGAT
ACTATACTCCATTTCTAAGAGGGAAGTAGATGATTTGGTTGAAAATTTG
GATGTCTCTCAGTGGAAGTTTATCTCTAAAGCAATTCAAAACTTATATC
CAAATGATATCGCCCAGAGGAATGAAATGTTGATTAAATTGGGAGACAG
GTTAAAGGAAAATGGTCATAGACAAGATTCTTTGACTTTGTACTTGGCT
GCCGGATCATTAGATAAGGTGGCTTCAATTTGGTTATCAGAATTTCCAG
ATTTGGAGGATAAATTGAAGAAAGATAATAAGACAATTTATGAAGCTCA
TTCCGAATGTCTAACTGAGTTCATTGAAAGATTCACCGTATTTTCCAAC
TTCATTAATGGAAGCTCTACCATTAATAATGAGCAATTAATTGCCAAAT
TTTTGGAATTTATCAACTTAACTACTTCCACAGGAAATTTCGAACTAGC
CACTGAATTCTTAAATAGTTTACCAAGTGACAATGAAGAGGTTAAAACA
GAAAAGGCACGTGTCTTGATTGCTTCCGGCAAATCATTACCGGCACAAA
ATCCTGCGACAGCGACGACCAGCAAAGCCAAGTATACAAACGCCAAGAC
AAATAAGAACGTTCCTGTACTACCAACTCCTGGAATGCCTTCTACTACT
TCTATTCCTAGTATGCAGGCACCATTTTATGGTATGACACCAGGCGCCT
CTGCAAATGCTCTACCTCCAAAGCCGTACGTTCCAGCAACCACCACTAG
TGCTCCTGTTCATACAGAAGGTAAATATGCGCCACCAAGCCAACCTTCG
ATGGCGTCACCTTTTGTTAACAAAACAAATAGCTCGACCAGATTGAATT
CTTTTGCTCCTCCGCCTAACCCATATGCCACTGCAACAGTTCCTGCAAC
GAACGTATCTACAACGTCGATTCCGCAAAACACTTTTGCTCCTATACAA
CCTGGTATGCCTATTATGGGCGACTATAATGCTCAATCTAGCTCTATTC
CTTCACAACCTCCAATTAATGCTGTATCGGGTCAAACGCCACATCTCAA
CCGTAAAGCCAATGATGGTTGGAATGATTTGCCTTTGAAGGTCAAAGAA
AAACCATCTCGTGCCAAGGCTGTATCTGTTGCCCCTCCAAATATCCTAT
CGACACCAACTCCATTAAATGGTATCCCTGCAAATGCTGCTAGTACCAT
GCCTCCGCCACCTCTTTCCAGAGCTCCCTCTTCTGTGTCAATGGTATCA
CCACCTCCTCTACACAAAAATTCTAGAGTCCCATCCTTGGTTGCAACTT
CTGAGTCACCAAGGGCATCCATATCAAATCCATACGCTCCTCCTCAATC
ATCACAACAATTCCCAATAGGTACTATTTCTACAGCAAACCAAACGTCA
AACACCGCTCAGGTAGCTTCATCGAACCCCTATGCTCCACCACCACAAC
AAAGAGTAGCAACCCCATTATCTGGAGGCGTGCCTCCAGCTCCGTTGCC
AAAGGCCTCTAATCCATATGCTCCAACTGCAACCACTCAACCCAACGGT
TCCTCCTATCCTCCAACCGGTCCGTATACTAATAACCATACCATGACCT
CTCCTCCTCCCGTTTTTAACAAACCTCCCACTGGCCCCCCTCCGATTAG
CATGAAGAAGAGAAGCAACAAGTTAGCTAGTATAGAACAAAACCCATCT
CAAGGTGCTACTTATCCTCCAACCCTTTCCAGCTCGGCCTCTCCATTGC
AGCCTTCTCAACCGCCAACTTTGGCTTCTCAGGTTAATACCTCCGCTGA
GAATGTCAGTCATGAAATTCCAGCTGATCAACAACCCATTGTCGACTTC
TTGAAAGAAGAACTGGCTCGCGTAACACCATTGACCCCAAAGGAGTACT
CCAAACAATTAAAGGATTGTGATAAACGATTAAAGATTCTTTTCTACCA
TTTGGAAAAGCAGGATTTATTAACCCAACCAACAATCGATTGTTTACAT
GACCTCGTCGCATTAATGAAGGAAAAGAAATACAAAGAAGCTATGGTCA
TCCATGCTAATATCGCTACAAACCATGCTCAAGAGGGTGGTAACTGGCT
GACAGGAGTGAAGAGGTTGATTGGCATAGCTGAAGCGACTTTGAATTAA
S. cerevisiae SSA4 (SEQ ID NO 40)
ATGTCAAAAGCTGTTGGTATTGATTTAGGTACAACCTATTCATGTGTTG
CTCATTTTGCAAACGATAGGGTTGAAATTATCGCTAACGATCAAGGTAA
TAGAACGACGCCTTCTTATGTGGCTTTTACTGACACAGAAAGGCTAATT
GGTGACGCTGCGAAGAATCAAGCTGCGATGAACCCACATAATACAGTAT
TCGATGCTAAGCGTCTGATCGGACGTAAATTCGATGATCCAGAAGTGAC
GAACGATGCTAAGCATTACCCATTCAAAGTGATTGACAAGGGAGGTAAA
CCGGTAGTGCAAGTGGAATATAAAGGCGAGACAAAGACATTTACTCCAG
AAGAAATTTCCTCAATGATCTTGACAAAGATGAAGGAGACTGCTGAGAA
CTTTTTAGGAACAGAAGTGAAAGATGCTGTAGTAACGGTTCCAGCCTAT
TTCAACGATTCACAAAGGCAAGCAACAAAAGATGCCGGTACAATCGCGG
GCTTGAACGTTCTTCGTATCATTAATGAACCTACAGCTGCCGCTATTGC
GTATGGGCTGGACAAGAAATCGCAGAAGGAGCACAACGTCTTGATCTTT
GATTTAGGTGGTGGTACTTTTGATGTCTCTCTGCTATCCATAGATGAAG
GTGTCTTTGAGGTTAAGGCTACTGCTGGTGACACTCACTTGGGTGGTGA
AGATTTCGATAGTAGGCTGGTTAACTTTCTAGCCGAGGAGTTCAAAAGA
AAAAATAAAAAGGATCTAACAACTAACCAAAGGTCCCTAAGGAGGTTAA
GGACCGCCGCTGAAAGGGCCAAGAGAACTCTGTCTTCGTCTGCTCAGAC
ATCTATAGAAATAGATTCATTATTTGAGGGTATCGATTTCTATACTTCC
ATTACAAGGGCAAGATTTGAAGAATTATGTGCTGATTTGTTTAGATCTA
CATTGGAGCCAGTGGAAAAAGTTTTGGCTGATTCAAAATTAGATAAGTC
ACAAATTGATGAAATTGTACTTGTTGGTGGTTCAACAAGAATTCCAAAA
GTACAAAAACTGGTTTCTGATTTTTTCAATGGTAAAGAACCAAACCGTT
CGATTAACCCTGATGAGGCCGTCGCTTATGGTGCTGCCGTACAGGCTGC
CATCTTAACGGGTGACCAGTCGTCGACGACCCAAGATTTACTGTTGCTG
GATGTTGCACCATTATCTCTAGGTATTGAAACTGCAGGTGGTATTATGA
CAAAGTTGATCCCAAGAAATTCGACTATCCCAACAAAAAAATCGGAAGT
GTTTTCCACCTACGCTGACAACCAACCTGGTGTGTTGATACAAGTTTTT
GAGGGTGAAAGGACAAGGACAAAAGACAACAATCTACTGGGTAAATTTG
AGTTGAGCGGTATTCCACCCGCTCCAAGAGGCGTACCACAAATTGAAGT
TACATTTGATATCGATGCAAATGGTATTCTGAACGTATCTGCCGTTGAA
AAAGGTACTGGTAAATCTAACAAGATTACAATTACTAACGATAAGGGAA
GATTATCGAAGGAAGATATCGATAAAATGGTTGCTGAGGCAGAAAAGTT
CAAGGCCGAAGATGAACAAGAAGCTCAACGTGTTCAAGCTAAGAATCAG
CTAGAATCGTACGCGTTTACTTTGAAAAATTCTGTGAGCGAAAATAACT
TCAAGGAGAAGGTGGGTGAAGAGGATGCCAGGAAATTGGAAGCCGCCGC
CCAAGATGCTATAAATTGGTTAGATGCTTCGCAAGCGGCCTCCACCGAG
GAATACAAGGAAAGGCAAAAGGAACTAGAAGGTGTTGCAAACCCCATTA
TGAGTAAATTTTACGGAGCTGCAGGTGGTGCCCCAGGAGCAGGCCCAGT
TCCGGGTGCTGGAGCAGGCCCCACTGGAGCACCAGACAACGGCCCAACG
GTTGAAGAGGTTGATTAG
S. cerevisiae SSE1 (SEQ ID NO 41)
ATGAGTACTCCATTTGGTTTAGATTTAGGTAACAATAACTCTGTCCTTG
CCGTTGCTAGAAACAGAGGTATCGACATTGTCGTTAATGAAGTCTCTAA
CCGTTCCACCCCATCTGTTGTTGGTTTTGGTCCAAAGAACAGATACTTG
GGTGAAACTGGTAAGAACAAGCAGACTTCCAACATCAAGAACACTGTCG
CCAACTTGAAAAGAATTATTGGTTTGGATTACCACCATCCAGATTTCGA
GCAAGAATCTAAGCACTTCACCTCTAAGTTGGTTGAATTGGATGACAAG
AAGACTGGTGCCGAAGTTAGATTCGCTGGTGAGAAACATGTTTTTTCAG
CTACTCAACTAGCTGCCATGTTCATCGACAAAGTCAAGGACACCGTCAA
GCAGGACACAAAGGCAAATATTACCGATGTTTGTATTGCTGTCCCACCT
TGGTACACCGAAGAACAACGTTACAACATTGCTGATGCTGCTAGAATTG
CTGGTTTGAACCCTGTTAGAATTGTCAACGACGTTACTGCTGCCGGTGT
TTCTTACGGTATCTTCAAGACTGATTTGCCTGAAGGCGAAGAAAAGCCA
AGAATTGTTGCCTTTGTTGATATTGGTCACTCTTCCTACACCTGTTCTA
TCATGGCCTTCAAGAAGGGTCAATTGAAAGTCTTAGGAACTGCCTGCGA
CAAGCATTTTGGTGGTAGGGACTTCGATTTGGCTATAACAGAACATTTC
GCCGATGAGTTCAAAACTAAATACAAGATTGACATCAGAGAAAATCCAA
AGGCTTACAACAGAATTCTAACTGCTGCTGAAAAGTTGAAGAAAGTTTT
GTCTGCTAATACTAATGCCCCATTCTCTGTTGAATCCGTCATGAACGAC
GTTGATGTTTCCTCTCAATTATCTCGTGAAGAATTAGAAGAATTGGTCA
AGCCATTGTTGGAACGTGTTACTGAACCAGTTACCAAAGCTTTAGCTCA
AGCCAAATTATCTGCTGAAGAAGTTGATTTTGTTGAAATTATTGGTGGT
ACTACTCGTATCCCAACATTGAAACAATCCATTTCTGAAGCCTTCGGCA
AGCCATTGTCCACCACTTTGAACCAAGATGAAGCCATCGCCAAGGGTGC
CGCCTTTATTTGCGCCATTCACTCTCCAACTCTAAGAGTTAGACCATTC
AAGTTTGAGGATATCCATCCTTACTCTGTCTCTTACTCTTGGGACAAGC
AAGTTGAGGACGAAGACCACATGGAAGTTTTCCCAGCTGGTTCATCCTT
CCCATCTACTAAATTGATCACTTTGAACCGTACGGGTGACTTTTCAATG
GCTGCTAGCTACACTGACATCACACAGTTACCACCAAACACTCCAGAAC
AAATCGCTAACTGGGAGATCACTGGTGTTCAATTACCAGAAGGTCAAGA
CTCTGTTCCTGTTAAGTTAAAGTTGAGATGCGACCCCTCTGGTTTACAC
ACAATTGAAGAGGCTTACACTATTGAAGATATTGAAGTTGAAGAACCTA
TTCCATTACCAGAAGATGCTCCAGAAGATGCTGAGCAAGAATTTAAGAA
GGTTACTAAAACTGTAAAGAAGGATGACTTAACCATCGTTGCACACACC
TTTGGCCTAGACGCTAAAAAGTTGAATGAATTAATTGAAAAAGAAAATG
AAATGCTTGCTCAAGATAAGCTAGTTGCTGAGACAGAAGACCGTAAGAA
CACTCTTGAAGAGTACATCTACACATTGCGTGGTAAGTTGGAAGAAGAG
AGCTAAGTACATTGCCAAATACGAAGAATTGGCTTCTCTAGGTAACATT
ATTAGAGGTAGATACTTGGCTAAAGAAGAAGAAAAGAAGCAAGCTATAA
GATCTAAGCAAGAAGCATCCCAAATGGCTGCTATGGCTGAAAAGTTGGC
TGCTCAAAGAAAGGCAGAAGCTGAAAAGAAGGAAGAAAAGAAGGACACT
GAAGGTGATGTTGACATGGACTAA

By screening a P. pastoris genome database (ERGO™, IG-66, Integrated Genomics) with the nucleotide sequences of the secretion helper factors isolated from Saccharomyces cerevisiae (SEQ ID NO 32 to SEQ ID NO 41) homologous nucleotide sequences in Pichia pastoris have been identified and are shown in Table 4 below.

TABLE 4
Homologous Pichia pastoris nucleotide
sequences(SEQ ID NO 42 to SEQ ID NO 51) and
respective ERGO ™ database information

BMH2 (SEQ ID NO 42); RPPA07190 -
Pichia pastoris (IG-66)
ATGTCAAGAGAAGATTCTGTTTATTTAGCAAAACTAGCTGAGCAAGCT
GAGCGTTATGAGGAGATGGTCGAGAACATGAAGACCGTCGCCTCTTCC
GGCTTAGAGTTGTCTGTCGAAGAGAGAAACTTGCTTTCTGTTGCATAC
AAAAACGTAATTGGAGCTAGAAGAGCTTCTTGGAGAATCGTCTCCTCA
ATTGAACAGAAAGAGGAAGCCAAGGGTAACCAATCACAAGTGTCTTTG
ATCAGAGAATACCGCTCCAAGATTGAGACCGAATTGGCCAACATTTGT
GAGGATATTTTGTCTGTTTTGAGTGAGCACCTTATTCCTTCTGCCAGA
ACTGGCGAATCCAAGGTCTTCTACTTTAAGATGAAGGGTGATTACCAC
CGTTATTTGGCCGAATTCGCTGTTGGTGACAAGCGAAAGGAAGCTGCT
AATTTGTCATTGGAGGCTTACAAGTCTGCCTCTGACGTTGCTGTTACG
GAGCTACCTCCAACTCATCCAATTAGATTGGGTCTGGCTCTGAATTTC
TCAGTCTTCTACTACGAGATTCTAAACTCTCCTGACCGCGCCTGTCAT
TTAGCCAAGCAAGCTTTCGACGATGCTATTGCTGAGTTAGAAACCCTA
TCTGAAGAATCTTACAAAGACTCCACTTTGATTATGCAACTGCTGCGT
GACAACTTGACTTTGTGGACCTCAGACATGTCTGAAACTGGACAAGAA
GAGTCATCCAATAGCCAAGATAAGACAGAAGCTGCTCCCAAAGATGAA
GAGTGAATA
BFR2 (SEQ IN NO 43); RPPA04523 -
Pichia pastoris (IG-66)
ATGGCTAGAAAGACATTGGCTGAAACATTGGCAGAATTGTCTCAACCA
GCGTCTGGAGATTTTGATATAGAAGACCAAGAAGGAGGAGCAGTACTT
GACTATGGAGATAATAGTTCTTTTGGCTCCGAGAGTGAAGAGGATAAA
AGTAACCACTATGTTAAAGTTGGCAAGTCAAGGATAAGAGAGAACGCA
GTTAAATTGGGAGGACAATACGAGGGAAAAAAGAGTAGTAGAGCCGAT
GTTTTTGGAGACGAGGACGATGAGGAGGAGGACGATGAGGATGTTGAA
CATTCGGAAACTGAAGATGCACTTTCGGTTTCAGGATCAGAGTCCGAA
TCGGATGAAAAAAATAGTGATCAAAGCCAAGGTGATTCTGAGAGTGAA
GAAGAATCTAACTCAGGTGAAGATCTAGACTACAAGAGATCAAAACTA
CAGCAACTTATAAGCTCCGAAAGGAAAACCATTGTAAACCAATTATCA
ACTTCCAATAAACAAGATGCACTGAAAGGGTTTGCAGTGTTGAATCAG
CAGATACAGTATGATCAATTGGTTGACCTCAGAATAAAATTACAGAAA
GGATTAGTAGCATCGAATGGTCTACCCATTAACAAAGAATATTACGAA
CAGAATAAAGCACCAAAGTCTTCCAAACACCTGGATAAGCTACAAGAT
AAACTATACAATTTATTGGATGTCACTTTAGAACTGAGAGGCAAGCTA
TTAAACAAAAGCAAGATTGTGAGCCAAGAGTTTCCCCCTATTCCAAGT
AAGAAACGTAGTTTACAGCATTATTTGGAGGAATCTTCCAAGTTGGAT
AACATAGTTAATGAATATAGAAGGAACGTCCTCGTTAAATGGTCTCAA
AAAGTCCAAAATGCTTCCGGAGCAACTGCTTTGAGCTCATCCAAATTC
AAGGCTATTAACCAAGATAGTTCGACTCAAGTGGACAACTATTTGGCA
GACATGGATAGATTAATCAAAAGAACCAGACTCAACAGAAGAAGCGTA
GTGCCATTAGGATACACCGAGACAGAAGAAGTAGTAGATGATGATGAA
TTGATCGACAACGATAAAGATAACAATGAGACCAAATACTTCAGCAAC
ATTGACCGATCTTTGAAGGAAAACAAATATATCTATGATGATGACGAT
TTCTATAGAGTTCTTCTGAACGATCTAGTCGATAAGAAAGTTTCTGAT
ACACAGAAGCTGACATCTACATCAACTGTTATTACATTTTCGAAATCC
AAATTGCATAAAAGTTATGAAAGAAAAGCGACTAAGGGTCGTAAGCTG
AGGTATACAGTTCAAGATCCATTATTGAATTTTGAAGCCTCCAACCCA
CATGCCTACAAGTGGAACGACTACCAAATTGACGAGTTTTTTGCGTCA
TTATTTGGGCAAAAGGTCAACATGAACGAGGATGAGCATAACGAAGAG
GTACAAGGTGAATCAGAAGGAGAGGACATTTTGAAGGATGATATCAAA
CTGTTTGGATAA
COG6 (SEQ ID NO 44); RPPA07651 -
Pichia pastoris (IG-66)
ATGGACTTTGTATATGAGTACTCAGATGCTACCCCTAGTGGCACATTT
GATGACCCATTGCCTGCAGAGCCCGAACCACCATTCAATTTGTCAAAC
TTAAACTCGTACAAAGATGATTTGACTAAAAAATTCTCCAAAATGAGC
ATTCTGAAAAGTCTGAAAAATGACACCAATTCAGTTGACGATGTCGAC
GACTCACAATCGATCTCCAATGACGGGCAGAGGGCTTATAAATACGCC
AATCAGTCTCTGGATCTGGTTAACCAGCACACCACTAATAAATCAATC
AGAACCACCAGCGATGAACAACCTTCGGTGTCCACTGTTTTGAGCAAC
AGACTGAGCAGAGTGCTCAATAATACTAATTACGACCCTTCAACCAAG
GAACTACTCTCCATTGTGGAGAAGAAAATAAAAGAAGATACGGCGCAT
GAATACGACAAAGTTACTGACCCAAGTTTTGTTGGAAACCTTGCTAGA
AGAAAGTTGCGTAACGACATTGAACATGATGTTGTAGATGCCAACTTC
AATTTCTTGAAACAATTGCAACCCTTAAGAAAGACCTTGGGCCAGATT
GAAAGTGACTTGAATGAAATGAACGAGCTCAACAATCAAATCACTGAA
AAGTTGTCCTCTAGAGTTGAAGATACCACTAGGTTGGATAATTCCATA
CACGAGTTGCATGCAACTTCCAAGATTATTTCCATCAAAAAGAAGCTT
TTGCAGAATTTCCAGAACCGCTATACTCTCTCCCATTTCGAAGCACAT
CAATTAGAGTTTGGTGAAATTGACGATTCCTTTCTAGAAATACTGAAA
AAAGCTGAGTCAATTCATGATGATTGTTCAATTTTGTTAACCATGGAG
AATGCTACTGTGGGTATTAATATTATGAACGACATGAAAAAGCTTTCC
AATAATGCCATCGACAGATTGTCGACATTTGTCACCAAACATTTTTCT
AGGTTAAGTTCGTCCAACAATACCTCCGCCTCCATAGAGGATAAGGCA
TTCCTGAAAAGATCTATACTCTTCATTTCCGAAAGATACCCGGAGCAG
CTCTCTGGAATCACCAACCAAATAGTCGAATCAAGGTCAAAGTCTTTG
CTTGACGAGTTCCAAATACAATTGAATGGTTATGCAGATTCAGCATCA
AGAAACGAAAGGGATGTTAATAAACCATTGTTCCTTTCCGCATATGAT
TCAGAAAGGTTTCTCGGAGATTTACTTGCTTATATTCATGGCACAATT
GTTAATGAAAGAGAAACCGTCGAAAGCTTGTTCAGTTTGCAAGATGAG
GAGAAAGATAATATCGTTTTGACAACACTCGTAGAGTCAATTGTTTCA
AAGAACATCGAATCTCTAGCTACCCCCCTGAATTTAAAGATTGAACAG
ATCATCAGAAATGAGTCTAAGCTGACAGCGATCCAAGCTTTTTATGAC
CTGCTCTCACTTTATTCCATGATGCTTGAAAAAACTTTAGGTTCTAAG
AATGCCCTTTTGAGTACAATCAACTCTTTAAAAGTTTCGGCTTTGGGT
AAGATTCAAAGTTCAATCAACATTAAACTTAAAAACATAGAGCGGACT
GCCAATGAGAGCATGTCATATTATAATGAAGATGAACAACTAGATGGT
ACAAACCACAACTTTGTTTCAGAAACTCATTACATTGAAGAAATCACA
CCTGAGCTAGCTGTGCCTGATTGGTTGATCAATTTCTATGGTGACGTA
CTTCCCATCTTTGATAATGAAAAGGTGACAAATGCCAAAGAACTGTAT
GAGGATTTACTCAAATACTGTTTTGAACAAATCATTCAACTTATCGAG
AAACAAATAGCTCAGAATAAATTGAATGATGCTAGAGAGATATTGATT
TTCAAATCAAACTGTTACGATTTTGTTTATTCCAAAATTGTGACCCTG
AACATCTTTAAGGAGAAACTGGATCGATTAGAGGTAATGATAAAGGAA
TGCGAATCAAAATTGACCGAAATTCAGTACACTTATCTTCTCAAACAA
TCAGGGTTATATGATATTCACAACCTTGTCAACATGATATCCTCAACT
AGGGAAGATTTCTTTGACGTCTCCGTTTATGAACCAATTACGGAGAAC
TCACTATTCAATGGTGACAAATTCAAAGAAATATCAGATCGCCTTCAA
GATTTTCTTCCAATTGCATTAATTGATTACCAAGAGGAGCGATTGTTG
TATCTATTACCTCCCACGCTTGTTAACTCTATCATTCAAAACTCCTCT
GTGGATTTTGTCAACTTTTATTTCAAATTATCGTTGATCGTGAAGGAA
TATTTGAAAGCCAGTGAAGGATGTCTCAGATGGGATGACATGGAGGT
COY1 partial (SEQ ID NO 45);
RPPA05747 - Pichia pastoris (IG-66)
AAAAGTTAAGTAATGAGTTGGTTAGCTACAGATCGATAACAAGAGGAC
ATGGTTAATTCAATTCAGGAACTGGAAGAGAAGCTTGCGTTTTCTCAA
AAGCAAGTAGAGCAGTTACAGCAGTTAAACCAGGATTTGGAGAAGGAG
ACTAGTGTGGAAAAATGGGATGCAATTTCAATGATTTCTGCCAGGCCG
GACACTTCAATACAGGACAATTCGTTGATTACAATGGTATCACAACAA
AGAGATCGATTTAAGCAAAGGAACAAAGATCTTGAAAAAGACGTTAGA
TTACAATTGAACAAAATTTCTGAGCTTCAAAGAAAGGTCCAATCACTT
TCTTCAGACAATAATCAATTATACGAGAGAATCAGGTTTTTGTCATCC
TATGACAGTAATAAGAATCAGTCCAAGGAGAGCCAGTCGGAAGAGTAC
TACAAGAGAAGTTACGAAGACAAATTGCATCCGATAGAACAATTTAGC
ATATTGCATCAGCCTGCACTTAATTGTGATGTCAATGACGATGTATGT
GATGAATATCCATAATGA
RPPA04443 - Pichia pastoris (IG-66)
AATTCATACCGTTGTCACGTAATCGCGGGGGTAGTGTGCATCGCATCG
TATTGGAGACATCTGTCTGTTTTCTTCCCTCACATCGAAATACAACTT
CACCATGACTGACGCTGACTTCCAAGTAGTATTTGAAGCGTGGCAAGC
TGTCGATCTACAGGGTGTTAAGAAGCTTGTAGATGATGAGGCAAAAGA
GATTGAAAGTTCGAAGTCTTCAAGTTTGGATCAAAGAAAGCAGTTGAG
TTTTAAGACGAAGGAGTTCAAGAAATTGGACGATGAGCGTAAATTGAC
ACAATGGAGGTCGTTGTTGAAGGAGTATCAAAACTACATTGATGATTT
GACCAAGGGAAATAATCGTGTTGTACAGACATTCTTGGAATTGCATAA
AGTAGTGGTGGATTTGAAGGATCCTACAAGTACTTTGAGCAAGGAGCA
AGAGACGAATACCGAATTACAGAAAGCTGTGAAAAAACTTTCCACAGA
ACTGAGGCATTCAGAACAACACTGGGCTTCCGAGAAGAAAGGATTGGA
AGAAAAATTTAACGTACGTAAAAGGGAAACGGAAGAGAAAAGTCTTGA
TCAGATTAAGACAGCCCAAACTGAAATAGTCCAATTGAGGGATGAGCT
GAAGCAAAAATCTTCAGAAAATGAAGAGCTTCAAGTGGTGATTGAAAC
CCTTGATGCCAAGCTAAAAAAGAACAGCCAAGGACAAAATAATGATGA
TACATACTCCAATTATGACATGTTAAATAGAGATTTGGAGTCCAATAA
ACTAAAGATCCTTGAATTGGAAAGGTTGAACAACTCTCTAAAGGAGGA
ATTAGCAAAGAAAGATGACAAAGCCTACCAGGAGAGGGTCACCGAACT
CGAAAAGGAGAGTGTGGAGTATCTCTCTTAAAG
CUP5 partial (SEQ ID NO 46);
RPPA09067 - Pichia pastoris (IG-66)
GTCCTGTTTATGCTCCATTCTTTGGATCCATTGGTTGTGCTGCGGCCA
TCATCTTTACCTGTTTTGGTGCCGCCTATGGTACTGCTAAGTCGGGTG
TAGGTATTTGTGCCACCTGTGTCTTGCGTCCAGACTTACTGATCAAGA
ATACAGTGCCTGTTATTATGGCTGGTATCATTGCTATTTATGGGTTGG
TGGTGTCTGTGTTGATCTCTTCATCGTTGCAACAGAAGCAGGCTTTGT
ATACTGGCTTTATCCAATTGGGTGCCGGTTTATCAGTTGGTCTGTCAG
GTCTGGCTGCTGGTTTTGCCATCGGAATTGTTGGTGATGCTGGTGTCA
GAGGTACTGCTCAACAGCCAAGACTTTTCGTCGGTATGATTCTGATTT
TGATTTTTGCTGAAGTTTTGGGTCTTTACGGTCTGATTGTTGCTCTTC
TACTGAACTCTAGAGCTTCCCAAGATGTCACTTGTTAAAGC
IMH1 (SEQ ID NO 47); RPPA04985 -
Pichia pastoris (IG-66)
ATGTTCTCAAAACTTTCCCAGTTATCCCAGAATTTAGGCGAAGAGCTC
TCTAGGATTAATGAGGAGGTTGCTGCCTCTAGAAGGAACCAACTAAAG
AAAAGCAGGGATTCGGAGAGGGATACAAAGTTCCTTAACATCAAAACT
CCCGATCCTGAAGCTCTACAACAACCGGGTCATGAGGTGAATGAAGGC
GCTGAAACCGAAACAGATGCTACTGAGTCAAAGGGCCAAGTGGTTCCA
AACACAAATATACACTTCAATGATCTGCCTATGGAGATTAGGGCCCGC
TTGAAAAAGTTTGCGAAATATGAGCAGAAATATCCGTTGTTGTTGGAC
GCTTACAAAACTGAGAAGGCCAAATCTGAAATAGTTCATGCTTTTGAA
TCAACTTTACAAGAAGTCACTCCTTTGCAGACAATTGGAGAAATTGAA
CAATTCAAAGACTTTATCAGCAATATGACCCAAAAGGCTAAATTAATG
GATGAAGAATTGAGAGCCAAAACTGGCGAGTTGAATGGCCTAAAAAAC
GAAGTGACAGAAATGAAAGAGAAATTGAAGGCTGTTCAAGGTGAGATG
AAAGCCAAGTCTGCTTTAGCAGAAGAATCTGCGATGAAAGCCGATCAA
CTTAGTGTGGATCTTGAACGAGTTGTGAGTGAGCTAGAAAATTTGAAA
AAGGAAAGGGAAGAGATTGTTACGGAGCGTGATGAGGCAACCAAGGAA
CGTGACGAGTCAACAAGAGAAAGAGATATCATTCTAGAAGAAGTCAAA
TCTAATAAAAATCAAGAACTTTTGGAGGAATACAAGTCTGAGTTAGAA
GAAGCAAAGAACGCTCTTGCATTGAGAACTGAGGAGATTGAAAATCTA
AACTTGAAGTTGGAGTCTGAAAAGTCGGCAAAGTTATCATTAGAGGGT
GTAGCAGATGAGCGAGATGGCCTTAAAGCAAAGTTAGAAGCGCAAACA
ACTTCCTTCCAGGAAGAATTAGACCAACTTTCTCAAGAACGGGATCGT
TTGAATTCTCAACTAACAATAGGAGAAAAGTCACAGATAGAAATTGAG
CAAGAAAAAAATGAGCTCAAGAGTCAGTACAATTCTGAGATTAAGTCA
TCGCTTAGTAAACTGGAATCCGTAATTAAAGAAAGAAATGAGCTACAA
CAGCAATTGGAATCTCAAGAGTCTTTAACTTTCGAAGTGGATAAGCTC
TCTAAAGAGAGAGATGAGCTAAGGATGCAGTTGGATAGAGAAAAAGAA
AATTCTGCAAAGGCTAGCATCACGCCCCAGAACTTTGAGGTTAAAACT
AAAGTTGAATCCAACAAGAACATTGAAGCACCTTTATCTGAAGAACTC
AGGCAAGTCACCAGAGAAAGAGATGAGCTAAAAGCTCAGTTGTTGCTC
ATTCAAAAGAACCCAGGACCAAGTAAGAAGACCAATGAAGGAAACCGT
AACTTAGAGCGGAACGGTGAAAAAAAGTCCCATGACCAAAATGGTGCT
GACGATGATCTTATCGAACGGAAGGGTGAAAGCGGTACAGATGATTCA
AAGGATAATCAGCTGAAACTAATAGAGCAGTCAACCACTATTACGATG
TTGAATGAAGAGATTGAAAACTTGAAAGATATGCTGCGTGACGTCGGA
GACGATCTTGTAGTGGCTAAAGACAAGCTTTCACAGGTCTCTGCTGTT
GATGAAAAAAAACAGCACGCTCTTGAAAGGGAACTGGAATCCTCAAAG
TTGAAGCTTGCTGAGATTGAAAAGGATTACAACGACGATAGAGTTGAC
CTCAAGAATGAATTGAAACTCGTTACCGAGGAAAAGGAAAATCTTGAA
CATGAGAATGAAACATTGAGTCAAAGCTTGACCGAGCTTGAAAAGCTG
AAACAAGAAGTCAAGGAGAAAGCTCAAGCCGTTCAGAATTATGAATCT
AAGTATTCGACACTATCGGATGAATTATCTTTAGTCCTCTCCAAACGA
GATGAATTGGAAAAAGACAAGGAAAGTTTCAGGCTGAAATTGAAAGAT
TTGGAACAGAAAAATTCCGAGACTGAACAACAAGAGGAATCGCAGAGA
ACTGGAACTGCAGAAATGGAAGAGGAGTTGCAGACTTTGAAAAGGGAG
CTAGAAGCAAGCTCCCAGCGCATTGAGGATTATAAGCAAAAGCAACTA
GAACTAGATGATGAAATATCTCTTGTACGTTCTCAAAAAGATGAATTA
CAGAAAACGATAACTAGCCTGCAAGAAGACCTGGGGAAAGAGAAAGAG
AATGTTAAGTTACTCCGTGAAAATATCATTGCCGAAGAAAAAGCCAAA
AATTCCCAAAAACTGGCAGAAAATGTGGCGCAGCTAGACAAATTCAAA
AAGCAGGAGATATCACTCAAGCTTGAGATTGCAAACCTTGAAAATCTA
AATGCTGAAAAAGGCTCAAAGATCAAAAGTTTGGAAGAGCATATCACT
TATCTTAATACAGAGAGGCAATCTAATTATGATGAGAATCAGAAACTG
ATCTCCCATACTAATGAGAAATGGAAGCAGGATTACACGGAGTTAGTC
ATAAAATTGAACAAGTGTCAGTCAGAGAACAATAGACTTACAAAAGAA
TTGAACGAATACAAAGATAAACTAAAAGATATGAACACCTCAAAGCTG
AACAGTAGCGAGACAATCGAGTCAATCAGAAGACAATGCGAAGAACTT
AAAATGATGAATAATGAATATTCTTTGAAGATTGAAAGTCTACATGAA
GAACTAAGTTCTTCGAGTTCAATTTTACAGGAACGTTCCAGAGAAATG
AATACTATACGTAAACTGCTAGCTGATACTGAGTCCAAATGTGACGAA
AGAATCAAACAGTTAAAAGCAAGAATTGATAGGTTAGAAGAAGAAAAG
GAGACGACTAGCCATGAAAGCTCTGTCCAGGCAAGAAAGCTGAGTAAA
ACAATCGACCAGTTAAAGAAAGGCAAGAATGAATTGTCAGTGCAGCTA
GAACAATGTAAGCTAGAGCTGGAACATCTGAAATCCGTCCCATCTAGA
GTGGATGTTGACAATAAAAACGGTGCTTCAAATGAAAACAGTGATGAA
AACCAATCTGATATTGAATCTGGAATTATCGAACAGCTCAGAAACTCG
CTAAAGGGATATGAAGAACAACTAAAACAATACCAAGATTCCAACGTC
TTACTCAAGAAGGTTAACGAAGAGCAGTTGCTGAAGTTCGAGAGACTG
CATTCAAATTTCAAGATTGTATCTAAACAATATAGAATGCTGAAAGAT
CAAAAGGACGAAGTCAATACGAGAAGTAGAAACAATTCAGTTATAAGT
TCAACGAGCGCGGGGAGTGATGAAAATGAGAGAGATAAAGTTGCCTAT
ATTAAGAACGTCCTTCTAGGATTTTTGGAACACAAAGATCAACGAGCT
ATGCTTTTTCCTGTAGTGAAGATGCTACTTATGCTGGACGATGATGAA
GAGAGAAGGT
KIN2 (SEQ ID NO 48); RPPA04639 -
Pichia pastoris (IG-66)
ATGGATAGAGAACAGGGTATTCTGCCACAGGATCCCTTCTCCAACTCG
GTGCATGTACCAAAGTTGAGAGCTTCTTCTGGTGGCCAGCCACAGAAG
CCTGTAATACAAAATTCTGCTCCTGCTACTGCTAGGATGCTTCGCAAT
GCAAGTTCAAGTACGTCAGCAGCTTTGTTGAAAGAATTAAACACACAT
GAACACTCTCAACGTCAACATACTCCACAGAAACAACCATCATTGGAT
GCCCCGGCAGCATTGGTTCCAGTTGAATCTGCCACAAAACAATTCCAC
CGAACCTCCATTGGAGACTGGGAATTTAGTAATACAATTGGGGCAGGC
TCGATGGGTAAGGTCAAAGTCGCCAAACATAGAGTCACTCACGAGGTA
TGTGCCATCAAAATAGTCATTAGGTCAGCCAAAATCTGGCAGAGAAAT
CACCAAAACGATCCAGAACCTGAAACTGAAGAAAAAAGAAAGAAGCTG
CGTGATGAATACAAGAAGGAATTGGAACGCGATGAACGTACTGTCAGA
GAGGCAGCACTAGGAAAAATAATGTACCACCCAAATATTTGTCGGTTG
TTCGAATGCTATACAATGTCTAATCACTACTACATGCTTTTTGAAATA
GTCCAGGGGGTACAGTTACTGGATTATATTGTTTCTCATGGCAAATTG
AAGGAAACACGCGTTCGCCAGTTTGCCAGAAGCATTGCTTCTGCTTTA
GATTACTGCCATTCTAATAACATCGTTCACAGAGATCTGAAAATTGAA
AACATAATGATTAACAAACAGGGTGAAATCAAGTTGATTGACTTTGGC
CTTTCCAACATGTATGATAGAAGAAATCTCCTGAAAACCTTTTGCGGC
TCCCTATACTTTGCAGCACCGGAGCTTTTGTCTTGCCGTCCTTACATT
GGTCCTGAAATTGATGTCTGGTCTTTTGGGGTTGTATTATTTGTCCTT
GTTTCCGGTAAGGTTCCCTTTGATGACGACAGCGTGCCAAAGCTTCAT
GCTAAAATCAAAAGAGGAAAAGTTGAGTATCCTGAGTTTATTTCACCT
TTATGTCATTCATTGCTATCTCAGATGTTAGTCGTTAATCCAGATCAT
AGAGTCACTTTGAAAGCTGCAATGGAGCACCCTTGGATGACCTTAGGA
TTTGCAGGGCCTCCATCAAACTATCTCCCTCAGCGGTCACCTATTGTA
TTACCGTTGGATTTAAGTGTAGTAAGAGAGATTGCAAATCTGGGTTTA
GGAAATGAAGAACAAATTGCTCGAGATATCACAAACCTGATCTCGAGC
AGAGAATATGAAGCGTGTGTTGAGAGGTGGAAACTTGATCAACAGAAA
GCTAATATCAAGGGCTATTCCGCGCGTGACGATTCTGCTATCATCGCC
TTCCACCCGTTACTTTCAACGTACTACCTCGTGGATGAAATGAGGAAG
AGGAAGCTAGCAAAAGGTGCTCTCAAGGGACAGACCTCGGTATTAGAC
ACTGTCAAGGTGTCTCCAGACATTCCAAAGACACCAGCTATTCCCCAG
AAACTAGAAACTACGGATGTGGAACAGCCATTGCTTGCCACTGTCCCA
CCTGCTTATACATCTCCGCATGGACAGCCAGCTGAACTGGAAGCGATG
ATTGAACCGGCACAGCCATTATCTAGTGCTCATCCTTTCGAGATGGAT
ATGACGCAGCAACAACATGCTAGCAGAAAGACCCATATCAAGCATGCT
CCAGAACGACAAGATCGTGGCGGCTATAATGTACACAAGAATAACTCT
GGTGGTCTTAACTCTTTATTCCGAAGACTCAGTGGAAAACGACCCCAT
AAGAATGAGGCTGAATGGGAGCCTTCATCTCCCCCACCTCAAGTTCAT
CCATTTTCAGTTAATGATGCGGACAGGACTTCAGTACGTGGCGTTTCA
CCAATTACTCAACCAGCTGCTGTGAAGAATGTGACCTCCAATAACTCC
AAAAACTACCTGGACCCTGTTGATGATAGTAAATTAGTTCGTCGTGTA
GGAAGTTTGAGAATTACCAACAAAGAAAAGCAACAAGTGACATCTGAC
TTTCCCCGACTGCCCAATTTTACGATTCCAGAGCAACCGCCTAAGAAT
GCTCCCATACCGATACATGCCCAACCTACCACTACAGGTACAACCTTT
CAATCCAATGATCATGAAATCAAAAAGAAGTTACAGGCTTCGACTAGT
CCAAACGAACAACGTGGGCCTCCAACATTGGCTCCTAGTCAACAGAGA
CGGCTACATCCCACTGCGAGAGCCAAGTCACTTGGCCATTCTCGCAAG
CAATCGCTTAATTTCAAATTCGGAGGACCAGCAAACAATCAATTACCT
GCGTTGCCTACTAAAGAAAATTATGATGTGTTTGAAGATGCCCAAATT
ACCGATAACAATTTATTAAACCCAGAAGGGAAATACTCTGCTAATACT
AACGTGCATATCAAACCAATGACAGAATCCCAAATTTTATTTGAGGCA
GAACATGCTCCACCTGGAACTATGCCCTCAGTTGAGTACCCCAGGACC
TTGTTTCTCAAAGGATTTTTCTCTGTTCAGACTACATCCTCGAAGCCG
TTACCTGTTATTCGATACAACATTATAGCAGCTCTCTGCAAACTTAAC
ATTCAATTCACTGAAGTTAACGGTGGGTTTGTTTGCGTTTACAGAAAA
ACTGAAAATTTACAAATTGGGGATATCAGATCTCCAGTTATAGAGTCA
AGAGTGACCGATGACACTGACTCCGATGTTGCAAACTCTTCCAAATTG
TCATCTTCGTCAACAGCCAATACCAGAGTCAATGTTATTGAGGATGAT
AGTTCATCGCCGTCCTCAGCAAGATTGAAACATCGCCGAAAGTTTTCT
CTTGGAAACGGAATCCTTAACCATATAAGGAAACCCACGCTTGACGGG
ACAGAATTTGATGACTACGATGCAACCGTAAATACCCCTGTTACTCCT
GCACCTGCAAATGTTCATTCTCGTTCATCGTCTTATCATACCGAGAGT
GATAATGAGTCCATGGAGTCGCTGCATGATATAAGAGGTGGCAGTGAT
ATGATCTTGAAAAATGTTCCAGAAAGAAATGCTAGACAGATAGACACA
GTCAAGGAAGAGGAAACAGATGATGATGATCTTGGTAGTATCAACGAA
GGATCAACACACCGTACACCTTTGAAATTTGAAATTCATATTGTCAAA
GTCCCTCTGGTTGGACTATATGGTGTGAGGTTCAAGAAAATTCTGGGA
AATGCTTGGATTTACAAAAGGTTGGCGTCAAAGCTGCTACAAGAATTG
AATTTATAGTTC
SEC31 partial (SEQ ID NO 49);
RPPA06211 - Pichia pastoris (IG-66)
ATGGTGAAAATAAGTGAAATAAAAAGTACTTCAACATTTGCATGGTCG
TCTGTAGACTCTAATGTCTTGGCTACAGGGACCTTGGCTGGGGCTGTT
GACGACTCATTCTCTACCACTTCGTCATTGGAACTTTGGGATGTCCTG
AACACCTCAGCTCCCATATTCCGAACCAATGTTGGTGCAAGATTTCAT
GATCTTGCGTGGAGTAATCCAATCTCTAAGTACCAGAGAGGACTACTT
GCAGGTGCTTTTGATAATGGAACAATTCAATTGTGGGATTCCTCATCA
TTGCTGAATGGATCATCTGACAGTTTAATAGAGCTAAAGAAACACACT
GCGCCTGTTAAAACAATATCTTTCAATCCTACAGAGTCACAGATATTT
GCATCTGGTGCTTCCAATGGCCAATTATTCATTTGGGATATAAATCAT
CTTTCAGAGCCTATTTCACCGGGTGCTTCTACTACCCCTATTAATGAC
ATAAACTCCATTGCTTGGAACTCCAAGATACGTCATATTTTGGCCTCT
GCTGGAACCTCAGGCTACGCATCCATTTGGGATTTAAAGACCAAGAAA
GAACTATTGAACTTGAGTTACACTGCTCCATCAGGTCAAAGAGCTAAC
TTAAGCACCGTTGCATGGCATCCTACTAATTCGACAAGTGTAATAACA
GCTTCTGATTCGGACGCTGTACCATTGATAATGACTTGGGATTTAAGG
AATACTAATGTACCTGTAGCTACTCTTGAAGGTCATCAAAAGGGTGTA
TTGTCCCTGGATTGGTGTTCGTGGGACTCAGAACTATTACTTTCTTCT
GGAAAGGATAACTCTACCCTATTGTGGAATCCCATCAGAGGCTCTTTG
TTAGCGGAATACCCAACCACCACTAATTGGGCCTTCAAGACCCGCTTT
TCTTCCAAGCTTCCTGACATTTTTGCAACCAGTTCATTTGATGGTAAG
ATAACGGTGCAGACCTTACAGGATACTACACCTGCAGAGGCTCAACAA
GCAAAAGCTATCAACGATGACGAATTCTGGGCAGACCTGTCCAACAGC
GATAAGAAACATCCTAATTTTTTACAACGTCAAACTCCGGCCTGGCTT
AAAGTGCCTTCCAGCGTTTCATTTGGATTTGGTGGAAAGATTGTAAAA
GTTTCCAAGGCCTCTGATAACCAGTCAATTGTTGTAATTGATAATTTC
ACAACTAACGATACGCTGGCCAAGTCCACTTCCCTTCTTGCAAGCACC
ATCAGCACAAACGATTACCAAACTCTTGTCGACGAAAAACTTCGTACC
GAAGCAAATAACCACGACTGGCAATTATTGAACGATCTATTAAAAGCG
GATGATGTGAAAGATTATTTCAGATTTCAGATTGTGGATCCTTCCGTA
TTGAAACATGACAAGTCTGAACAAAAGGTTGAAAACGGACAAGACATA
TTTGAAAACATTGAGCAAACTGATGAAGACTTTTTCAACAATCTTGAA
AGGGAAAAGAATTCAGTTTCTGTCAACATTCCATCATACTCTCCAACT
GCTCTCAGCCAGGGACTAATCCAGGAGGCTCTAGTATTGGCTTTAGGT
GCATCTGAATCATTACAGGCTAAAGTTAGGAATGCCTATTTTAACCAA
ACGCAAAAGTCCTCTCTACCAAGATTGATTTACAGTGCTACTGCTAAC
GATGTTAATGACCTCGTTGCTAATGGTACTATCTCTGGTTGGAGGGAT
ATAGCAGCTGCTATTTTTGCTTACTCTACGGAGAAAGAAGAGTTTTCA
AAGTTCATTGTGGAACTAGGTGATAGGCTATTAGCCAGTTCCCTTTCA
GATAGACGCTCTGATGCTCTGCTTTGCTTCCTTGCTGGTGGTGCGCTC
AACAAGGCGTCTACAATTTGGAATGCCGAGTTGAGTTCTCGTGAAGAG
GTTCTCAAATCTGAAAACCCTCAGCTCTCATCTTATGAAGCTCATAAT
ATTGTGTTGACTGAGTTTGTTGAAAAAATTGCCGCATTCAAGTATGCA
TTAAGGATCAGCAATAAGTTCAGTGGGCAGGGCGTTAATACGTTGAAC
AATTCATTCCTAGAGTTTGCTTCTTTGGTGTCATCTCAAGGGCAATTT
GATTTGGCCTTGAACTTATTGGAGAACTTATCTACCGAGGATGAAGAC
ATTAAACTTGAGATAAAGCGAATCTCAACAGCATCAGGAAAAACTCTT
TCCAGTA
RPPA07281 - Pichia pastoris (IG-66)
TCCTCCCNCGCTCACCCCTCCCGNCCTTCCCCTATCGCATTTCCAAAG
GTCTTCTCGTGGAGGCTCATTCTCCGTTCCACCTCCTAATCCATACGT
AGGTAGCTCAGTGAATGGGAATGGAGGAGTGCACGGAGGCGCACCAGC
TATCCCTGTTGCCAACAACCCTTATGCTAACAACAATCAAAATGCATC
ATATGGCCAAGCAAACGGTCCACTAAATGGATTTGTACCTCCGCCACC
AATGCCTGAGAAAATGGGAGGACTTTCCTCACAGAATTACCCCAAGAG
AGCAGCAAGTAGAGCAAATAGCACTGCTGGATATGCGCCATCACTAAG
ATCGCCCAGTGTGCAACAATTTCAACCACCACCACCTCCGGCACTAGC
TCAACATGTGCAACCGCCACCACCTCCTGAACTAGTTCAGCAGGTACC
TCCACCCGCGCCGTCTGTACAACACCAAGTATCACAAGGATCTCAAGG
ATCTCAAGGTGGGCCCCCTGCACAACAACAGACCAGATTTCCCAGTGG
AGATAGATCACATATAAGTGATGAGGCTTTCCCTATTTATGAGTACCT
GAGTAAGGAGTTGGAAAATGTTAAGCCTAAGATTCCAGAAAGATTTAC
CAAACAACTCGTAGACGCTGAGAAGAGATTGAATATCTTGTTTGATCA
TTTGAATAACAATGAGCTGTTAACCGCTCCTACGATTACACTGTTGTC
TAATCTTTCAAAGTCTCTAGCTGACCATGACTTTAAGACTGCTGAATC
GTTACTGATTCAAATTACTACCATTCATAACAACGAGGCAGGAAACTG
GAGCGTTGGTGTGAAACGTCTTATCCAGATGTCCTCGGCTCTGAGTAG
CTAAGAA
SSA4 (SEQ ID NO 50); RPPA10651 -
Pichia pastoris(IG-66)
ATGGGTAAATCAATTGGAATTGATTTGGGTACCACATACTCTTGTGTG
GCACATTTTGCTAATGATCGTGTTGAGATCATAGCTAACGACCAAGGT
AACAGGACGACTCCATCGTTCGTCGCCTTTACCGACACTGAAAGATTG
ATTGGTGATGCTGCAAAGAACCAAGCTGCCATGAATCCAGCTAACACT
GTTTTCGATGCCAAACGTTTAATCGGTAGAAAATTCGACGACCCGGAA
ACTCAGGCCGATATTAAGCACTTCCCTTTCAAAGTTATCAACAAGGGG
GGAAAGCCTAATATCCAAGTCGAATTTAAGGGTGAGACTAAGGTTTTC
AGCCCCGAAGAGATTTCCTCCATGGTTCTAACAAAAATGAAGGATACT
GCTGAGCAGTATTTGGGTGAGAAAATCAACGATGCAGTTGTCACTGTT
CCTGCTTACTTCAATGACTCTCAAAGACAAGCCACCAAGGATGCTGGT
TTGATTGCTGGTTTGAACGTTCAAAGAATCATTAATGAGCCCACCGCT
GCCGCAATTGCTTACGGGTTGGACAAGAAGGATGCAGGCCACGGTGAG
CACAACATTCTAATCTTCGATCTAGGTGGAGGAACTTTCGATGTTTCT
CTACTATCTATTGATGAGGGTATTTTCGAAGTCAAGGCCACCGCAGGT
GACACCCACTTGGGTGGTGAGGACTTCGATAACAGATTAGTCAACCAC
TTTATCGCCGAGTTCAAGAGAAAGACCAAGAAAGATCTTTCTACAAAC
CAGAGATCCCTTAGAAGACTAAGAACCGCTTGTGAGCGTGCAAAGAGA
ACTTTGTCTTCTTCTGCTCAGACCTCCATCGAGATTGATTCTTTGTTC
GAGGGTATCGACTTCTACACCTCGATCACTAGAGCTAGATTCGAGGAG
CTCTGTGCCGACTTGTTCAGATCCACCATCGAGCCTGTTGAGAGAGTC
TTGAAAGACTCCAAGTTGGACAAATCTCAAGTTCATGAGATTGTTTTG
GTTGGTGGTTCTACCAGAATTCCAAAGGTTCAGAAATTAGTTTCTGAC
TTTTTCAATGGTAAGGAGCCAAACAAGTCCATCAACCCAGACGAAGCC
GTTGCATATGGTGCTGCTGTCCAAGCAGCTATTTTGTCTGGAGATACT
TCTTCCAAGACACAAGACTTGTTATTGCTGGATGTTGCTCCTCTATCT
TTGGGTATTGAAACCGCTGGTGGTATCATGACCAAGCTGATCCCAAGA
AACTCCACAATCCCAGCCAAAAAGTCAGAAATCTTTTCGACATATGCT
GACAACCAACCAGGTGTTTTGATTCAAGTCTTTGAAGGTGAGAGAACT
AGAACCAAGGACAACAACCTGTTGGGTAAGTTTGAACTTTCTGGTATT
CCTCCTGCTCCAAGAGGTGTTCCTCAAATTGAGGTCACCTTCGATATG
GATGCCAACGGTATTTTGAATGTATCTGCTGTTGAGAAGGGTACCGGT
AAGACTCAAAAGATTACTATTACCAACGATAAGGGAAGATTGTCCAAG
GAAGACATCGAGAGAATGGTTTCTGAAGCTGAAAAATTCAAGGATGAA
GACGAGAAGGAAGCCGAGAGAGTTGCTGCCAAGAATGGCTTGGAATCA
TATGCTTACTCTCTGAAGAACTCTGCAGCTGAATCTGGATTCAAGGAC
AAGGTTGGAGAGGATGATCTTGCCAAGTTGAACAAGTCAGTTGAAGAG
ACAATATCTTGGTTAGATGAGTCACAATCTGCTTCCACAGACGAGTAC
AAGGACAGGCAAAAGGAATTGGAAGAAGTTGCTAACCCAATAATGAGC
AAGTTCTATGGAGCTGCTGGTGGAGCTCCTGGTGGAGCTCCTGGTGGC
TTCCCTGGAGGTTTCCCTGGCGGAGCTGGCGCAGCTGGCGGTGCCCCA
GGTGGTGCTGCCCCAGGCGGAGACAGCGGACCAACCGTGGAAGAAGTC
GATTAA
SSE1 (SEQ ID NO 51); RPPA10049 -
Pichia pastoris (IG-66)
ATGAGTGTTCCATTTGGAGTAGATCTAGGTAACAACAACACTGTGATC
GGTGTTGCCCGTAACAGAGGTATTGATATTCTTGTCAATGAAGTCTCT
AATCGTCAGACCCCCAGCATTGTCGGATTTGGCGCTAAGTCTAGAGCC
ATCGGGGAATCAGGAAAGACCCAACAGAACTCTAACTTGAAGAATACC
GTTGAACATTTGGTCCGTATTCTCGGGCTTCCTGCAGACTCTCCTGAC
TATGAAATTGAGAAGAAGTTCTTCACTTCGCCCCTGATTGAGAAGGAC
AATGAGATCCTGTCTGAAGTTAACTTCCAAGGTAAGAAGACTACCTTC
ACACCCATTCAGCTGGTTGCCATGTACCTGAACAAGATTAAGAACACT
GCCATAAAGGAAACAAAGGGAAAGTTCACTGATATCTGTCTTGCTGTC
CCTGTTTGGTTCACCGAGAAACAGAGAAGTGCTGCTTCCGATGCTTGT
AAGGTTGCTGGTCTGAACCCAGTTAGAATTGTCAACGACATCACAGCT
GCTGCAGTTGGATATGGTGTCTTCAAGACTGACCTACCAGAGGATGAA
CCCAAGAAGGTTGCAATCGTTGATATAGGCCACTCTACCTATTCTGTT
TTGATTGCTGCTTTCAAGAAAGGTGAGCTGAAAGTGTTAGGATCTGCT
TCTGACAAGCATTTCGGTGGTCGTGATTTCGACTATGCCATCACCAAG
CACTTTGCAGAGGAGTTCAAGAGCAAATACAAGATTGATATCACTCAA
AATCCTAAGGCTTGGTCTCGTGTTTACACTGCTGCCGAAAGGTTGAAG
AAGGTTTTGTCCGCTAACACTACAGCTCCATTCAATGTTGAATCTGTT
ATGAACGACGTTGATGTTTCTTCTTCGCTGACTAGAGAGGAGTTAGAA
AAGCTGGTGCAACCATTATTAGACCGTGCTCATATTCCCGTTGAGCGT
GCTCTGGCCATGGCAGGTCTCAAGGCTGAAGATGTGGACACTGTTGAG
GTTGTCGGAGGTTGTACTCGTGTTCCAACCTTGAAAGCTACTCTATCT
GAAGTCTTTGGAAAGCCCTTATCTTTCACTTTAAACCAAGATGAGGCA
ATTGCTCGTGGTGCAGCTTTCATCTGTGCAATGCACTCCCCTACACTT
AGAGTTCGTCCATTCAAGTTTGAGGACGTTAACCCTTACTCTGTGTCA
TATTATTGGGACAAAGATCCTGCCGCTGAGGACGATGACCACTTAGAG
GTCTTCCCAGTGGGTGGTTCTTTCCCATCAACTAAGGTGATCACACTT
TACCGTTCACAAGATTTCAACATTGAAGCCCGCTACACGGACAAGAAT
GCACTTCCAGCTGGCACTCAGGAGTTCATTGGCAGGTGGAGCATCAAG
GGTGTTGTTGTCAATGAAGGTGAAGATACTATCCAGACTAAGATTAAG
CTGAGAAATGATCCATCTGGTTTCCATATCGTCGAATCTGCTTACACA
GTCGAGAAGAAGACTATTCAAGAGCCAATCGAGGATCCAGAAGCTGAT
GAAGATGCAGAACCTCAGTACAGGACAGTTGAGAAGCTCGTCAAAAAG
AACGACTTGGAGATTACTGGACAGACACTCCACCTACCAGATGAGCTA
TTAAACTCTTATCTTGAGACAGAGGCTGCCTTAGAGGTCCAAGACAAA
CTTGTTGCAGACACCGAGGAGCGCAAGAACGCTCTGGAGGAGTACATT
TACGAGCTTAGAGGTAAGTTGGAAGACCAGTACAAGGAGTTTGCTAGC
GAACAGGAAAAAACCAAGCTTACAGCTAAGCTAGAGAAAGCTGAGGAA
TGGCTTTACGACGAAGGTTATGATTCTACTAAAGCTAAGTACATTGCT
AAATACGAAGAGCTTGCCTCCATTGGAAATGTTATCCGAGGTCGTTAT
CTTGCCAAAGAGGAGGAGAAGAAACAAGCTATCCGTGAAAAGGAAGAA
TCTAAGAAGGCTTCTGCTATCGCTGAAAAGATGGCTGCCGAGCGTGCT
TCTCGTGAAGCTGCTGGTTCTACAAATGAACAAGCCCAGAAGAATGAA
GAAAACACCAAAGATGCCGACGGTGATGTTTCTATGAACCAAGATGAG
CTAGATTAAACT

### Example 3

### Cloning of the Vector Backbone of pPuzzle

For construction of the novel vector system pPuzzle a 2884 bp fragment carrying an origin of replication and a selection marker for E. coli (AmpR cassette) was amplified from a common used cloning vector pBR322 (Fermentas Life Science, Germany, #SD0041 pBR322 DNA) by PCR. Two non-template coded NotI restrictions sites were added by using the forward primer pBR322_FOR_NotI and the backward primer pBR322_BACK_NotI. This PCR fragment was used as a shuttle supplying a temporary origin of replication and a selection marker for amplifying an artificial multiple cloning site in E. coli. A 244 bp synthetic DNA fragment (synthesised and subcloned in the EcoRV site of the pUC57 plasmid by GeneScript Corp. Piscataway, N.J. 08854 USA) was cut with NotI and ligated with the NotI and alkaline phosphatase treated shuttle fragment and amplified in E. coli. The resulting product was called pBR322½artMCS. To generate pBR322½artMCS_ORI, a 670 bp fragment carrying the origin of replication from a commercial available cloning vector pUC19 (Fermentas Life Science Germany; #SD0061 pUC19 DNA; bases 812-1481) was amplified by PCR using the forward primer pUC19ORI #1-SacI and the backward primer pUC19ORI #2-SacI and cloned in the unique SacI site of pBR322½artMCS.

To generate the vector backbone of pPuzzle (see FIG. 1), the ampicillin resistance gene (PCR amplified from pUC19 with primers ampR#1HindIII and ampR#2HindIII) is cloned into the HindIII restriction site of pBR322½artMCS_ORI, the resulting plasmid is cut NotI and religated.

In a further cloning step the transcription terminator of the cytochrome c gene from S. cerevisiae (a 276 bp fragment of the 3′ region of the Cytochrome c, isoform 1 CYC1 gene from S. cerevisiae chromosome X bases 526663-526937) was amplified by PCR (forward primer cyc1TT_new_FOR_BamH1 and reverse primer cyc1TT #2-AgeI) for genomic DNA and inserted into the BamHI and AgeI (alkaline phosphatase treated) site of pBR322½artMCS_ORI resulting in a vector called pBR322½artMCS_ORI_cyc1TT.

### Example 4

### Construction of a pPuzzle_zeoR_eGFP Expression Vector

The zeocin selection marker for E. coli and P. pastoris consists of the ORF of the Sh ble gene from Streptoalloteichus hindustanus under the control of the TEF1 (translational elongation factor 1) promoter from S. cerevisiae and an artificial E. coli promoter sequence EM7. The Sh ble gene is flanked by a transcription terminator of the cytochrome c (CYC1) gene from S. cerevisiae. The TEF1 promoter (5′ promoter region of TEF1 alpha of S. cerevisiae chromosome XVI bases 700170-700578) was amplified by PCR from S. cerevisiae genomic DNA using the forward primer zeoR_neu_{—}#1_kpn1 (adding a non-template coded Kpn I site) and the reverse primer TEF1_back:_{—}#1. An artificial E. coli EM7 promoter sequence and an NcoI restriction site were added to the 3′ end of the TEF1 promoter by primer extension using the forward primer zeoR_neu_{—}#1_kpn1 and the reverse primer TEF1_back:_{—}#2NocI. The resulting PCR fragment was treated with NcoI and fused to the NcoI site of the 5′ end of the Sh ble ORF. The Sh ble ORF was amplified by PCR using the forward primer Sh ble_FOR_{—}#1_NcoI (adding a non-template coded NcoI site) and the reverse primer Sh ble_back_{—}#2_AatI (adding a non-template coded AatI site) from a pUT737 plasmid (Cayla Toulouse, France pUT737 catalog # VECT 7371). The product of this fusion was used as a template for PCR (forward primer zeoR_neu_{—}#1_kpn1 and reverse primer Sh ble_back_{—}#2_AatI) resulting in a 893 bp fragment.

The transcription terminator of the cytochrome c (CYC1) gene from S. cerevisiae (Cytochrome c, isoform 1 gene from S. cerevisiae chromosome X bases 526663-526937) was amplified by PCR from genomic DNA using the forward primer cyc1TT_FOR_{—}#1_aat1 (adding a non-template coded AatI site) and the reverse primer cyc1TT_neu_back_Kpn1 (adding a non-template coded Kpn I site), treated by AatI and fused to the AatI treated 893 by hybrid of TEF1 promoter and Sh ble ORF. The zeocin cassette of the final size of 1170 by was amplified by PCR using the forward primer zeoR_neu_{—}#1_kpn1 and the reverse primer cyc1TT_neu_back_KpnI. The PCR product was purified by agarose gel electrophoresis and the fragment of the correct size was used as a template for a second PCR. The second PCR fragment was treaded by KpnI cloned in the KpnI sites of pBR322½artMCS_ORI_cyc1TT vector resulting in a vector called pBR322½artMCS_ORI_cyc1TT_zeoR.

For integration of the pPuzzle vector system in the genome of P. pastoris it was decided to use a target sequence in the 3′ area of the AOX1 gene of P. pastoris. Two 400 bp fragment called AOXTTpart1 and AOXTTpart2 (sequences from Integrated-Genomics, Chicago USA, ERGO database, P. pastoris IG66 Contig 1471 bases 52189-52588 and 52589-52979) were amplified by PCR from genomic DNA of P. pastoris. By using the forward primer 5_AOX TT #1 HindIII/NotI and the reverse primer 5_AOX TT #2 AscI/BamHI non-template coded HindIII and NotI restriction sites were added to the 5′ side and AscI and BamHI restriction sites to the 3′ side of the fragment AOXTTpart1. For adding a 5′ BamHI site, a 3′ NotI site and a 5′ EcoRI site to the fragment AOXTTpart2 the forward primer 3_AOX TT #3 BamHI and the reverse primer 3_AOX TT #4aNotI/EcoRI were used. For assembling AOXTTpart1 and AOXTTpart2 according to their orientation in the genome the fragment AOXTTpart1 was subcloned in the EcoRV site of pSTBlue-1 using the Novogen Perfectly Blunt® Cloning Kits, pSTBlue-1 (Merck Biosciences, Germany). A 500 bp fragment was amplified by PCR using the forward primer T7 and the reverse primer 5_AOX TT #2 AscI/BamHI. This fragment was cut by BamHI and ligated with the BamHI treated AOXTTpart2 fragment. The ligation mixture was used directly as a template for PCR with T7 as forward primer and 3_AOX TT #4NotI/EcoRI as reverse primer. The fragment of the correct size (˜900 bp) was purified by agarose gel electrophoresis and used as a template for a second PCR with 5_AOX TT #1 HindIII/NotI and 3_AOX TT #4NotI/EcoRI. The presents of the AscI restriction site in the middle of the PCR fragment was checked by AscI endonuclease digest of the resulting 800 bp fragment called AOXTTpart1+2 To get ride of the pBR322 shuttle in the pBR322½artMCS_ORI_cyc1TT_zeoR vector it was cut by NotI and the 2270 bp vector backbone of the pPuzzle_zeoR was separated from the 2884 by pBR322 shuttle fragment by agarose gel electrophoresis treated with alkaline phospatase and ligated with the NotI treated PCR fragment AOXTTpart1+2. The resulting vector was called pPuzzle_zeoR_AOXTT.

Starting from the pPuzzle_zeoR_AOXTT vector backbone an enhanced green fluorescent protein (eGFP) gene was inserted into the MCS using the restriction sites SbfI and SfII. The eGFP gene (718 bp) was amplified by PCR e.g. from the vector pcDNA™ 6.2n-EmGFP-DEST (Invitrogen Austria). Two non-template coded restriction sites Sbfl and Sfil were attached by primer extension using the forward primer eGFP#1AarI/SbfI and the reverse primer eGFP#2Sfil. The Sbfl and Sfil treated PCR product of eGFP was inserted into the alkaline phosphatase treated Sbfl and Sfil sites of pPuzzle_zeoR_AOXTT. The resulting vector was called pPuzzle_zeoR_eGFP.

In Table 5 the PCR primer sequences used in the cloning procedures of Example 3 and 4 are summarized.

TABLE 5
PCR primers for cloning of pPuzzle_zeoR_eGFP
(SEQ ID NO 52 to SEQ ID NO 74)

pBR322_FOR_Notl (SEQ ID NO 52):
5′-AATAGCGGCCGCGCATCTCGGGCAGCGTTGGGTCCTG-3′
pBR322_BACK_Notl (SEQ ID NO 53):
5′-GATTGCGGCCGCGACGTCAGGTGGCACTTTTCGGGGAAAT-3′
puc190RI #1-Sacl (SEQ ID NO 54):
5′-GATCGAGCTCTGAGCAAAAGGCCAGCAAAG-3′
puc19ORI #2-Sacl (SEQ ID NO 55):
5′-GAAAGAGCTCCCGTAGAAAAGATCAAAGG-3′
ampR #1 Hind III (SEQ ID NO 56):
5′-GCCGAAGCTTACAATAACCCTGATAAATGC-3′
ampR #2 Hind III (SEQ ID NO 57):
5′-GCCGAAGCTTAAATCAATCTAAAGTATAT-3′
cyc1TT_neu_FOR_BamH1 (SEQ ID NO 58):
5′-CAATGGATCCCCTTTTCCTTTGTCGATATCATGTAATTAGTT-3′
cyc1TT #2-Age I (SEQ ID NO 59):
5′-GTGGACCGGTAGCTTGCAAATTAAAGCCTTCGAG-3′
zeoR_neu_#1_kpn1 (SEQ ID NO 60):
5′-GATCGGTACCCACACACCATAGCTTCAAAATGTTTCTACTCCT-
3′
TEF1_back:_#1 (SEQ ID NO 61):
5′-TACTATGCCGATGATTAATTGTCAACACCGCCCTTAGATTAGATT
GCTATGCTTTCTTTCTA-3′
TEF1_back:_#2_Nco1 (SEQ ID NO 62):
5′-TTGGCCATGGTTTAGTTCCTCACCTTGTCGTATTATACTATGCC
GATATACTATGCCGATGATTAATTGTCAACACCGCCC-3′
Sh ble_FOR_#1_Nco1 (SEQ ID NO 63):
5′-TAAACCATGGCCAAGTTGACCAGTGCCGTTCCGGTGCTCACCG-
3′
Sh ble_back_#2_aat1 (SEQ ID NO 64):
5′-TCCGAGGCCTGGGACCCGTGGGCCGCCGTCGGACGTGTCAGTC
CTGCCTCTCGGCCACGAAGTGCACGCA-3′
cyc1TT_FOR_#1_aat1 (SEQ ID NO 65):
5′- TCCCAGGCCTCGGAGATCCGTCCCCCTTTTCCTTTGTCGATAT
CATGTAATTAGTTATGTCA-3′
cyc1TT_neu_back_Kpn1 (SEQ ID NO 66):
5′-ACATGGTACCTGCAAATTAAAGCCTTCGAGCGTCCCAAAACC
TTC-3′
kanR #1-Kpn I (SEQ ID NO 67):
5′-CCGAGGTACCGACATGGAGGCCCAGAATA-3′
kanR #2-Kpn I (SEQ ID NO 68):
5′-CCGAGGTACCAGTATAGCGACCAGCATTCA-3′
5_AOX TT #1 HindIII/Notl (SEQ ID NO 69):
5′-GATTAAGCTTGCGGCCGCAGAGGATGTCAGAATGCCATTTG
CCTG-3′
5_AOX TT #2 Ascl/BamHI (SEQ ID NO 70):
5′-GATTGGATCCGGCGCGCCGATACTCGAGAATTATGGCTTAATC
AAGTG-3′
3_AOX TT #3 BamHI (SEQ ID NO 71):
5′-GATTGGATCCTATGATTGGAAGTATGGGAATGGTGATACC-3′
3_AOX TT #4 Notl/EcoR I (SEQ ID NO 72):
5′-TAAAGAATTCGCGGCCGCAGCAACGTTGTCACTGAAGTTGGCA
TCA-3′
eGFP#1 Aar I/Sbf I (SEQ ID NO 73):
5′-GATCCACCTGCAGGCCATGGTGAGCAAGGGCGAGGAGCTG
TTCA-3′
eGFP#2 Sfil (SEQ ID NO 74):
5′-GGATGGCCGAGGCGGCCTTACTTGTACAGCTCGTCCATGCCGA
GAG-3′

### Example 5

### Comparative Yeast Promoter Activity Studies in P. pastoris

a) Amplification and Cloning Strategy of Promoter Sequences from P. pastoris:

To identify novel promoter sequences for use in a strain of the genus Komagataella for recombinant expression of a heterologous protein the normalized signals of all measured genes of trypsinogen producing and non-producing cells, respectively, obtained from the DNA microarray hybridisation described in Example 1 were ordered by their relative expression levels. Further the relative expression level of each measured gene was compared between trypsinogen producing and non-producing cells. From these data the 23 genes with the highest expression level in trypsinogen producing and non-producing cells were considered for further analysis. A listing of the genes selected for further analysis is found in Table 6. Further, only such genes for which genomic sequence data were available have been included in the selection. The promoter sequences of these 23 potential interesting genes (up to 1000 bp of the 5′-non coding region of the respective genes) were identified using a P. pastoris genome database (ERGO™, IG-66, Integrated Genomics) and amplified from P. pastoris by PCR. Additionally, the well known promoter sequences of AOX and of GAP were amplified via PCR from P. pastoris for comparative reasons (primer and primer sequences see Tables 6 and 7). In 25 final cloning steps the 25 promoters (including the two control promoter sequences) obtained from P. pastoris were inserted upstream of the start codon of the eGFP gene using the ApaI and the SbfI restriction site of the multiple cloning site of the vector pPuzzle_ZeoR_eGFP or in case of the promoter of FET3pre using the ApaI and the AarI restriction site (see Table 6).

TABLE 6
Overview of the genes, the PCR primers used for amplification of
the promoter sequences, the restriction enzymes used for cloning of the
promoter sequences and the fragment length of the promoter sequences
from P. pastoris

| | | | Cloning | | Frag- |
|---|---|---|---|---|---|
| | | | enzyme | | ment |
| gene | 5′ primer | 3′ primer | 5′ | 3′ | length |
| AOX | Paox #1 Apa I | Paox #2 Sbf I | Apa I | Sbf I | 1000 bp |
| GAP | Pgap #1 Apa I | Pgap #2 Sbf I | Apa I | Sbf I | 480 bp |
| GND1 | Pgnd1 #1 Apa I | Pgnd1 #2 Sbf I | Apa I | Sbf I | 1000 bp |
| GPM1 | Pgpm1 #1 Apa I | Pgpm1 #2 Sbf I | Apa I | Sbf I | 1000 bp |
| HSP90 | PHSP90 #1 Apa I | PHSP90 #2 Sbf I | Apa I | Sbf I | 1000 bp |
| KAR2 | Pkar2 #1 Apa I | Pkar2 #2 Apa I | Apa I | Sbf I | 1000 bp |
| MCM1 | Pmcm1 #1 Apa I | Pmcm1 #2 Sbf I | Apa I | Sbf I | 1000 bp |
| PET9 | Ppet9 #1 Apa I | Ppet9 #2 Sbf I | Apa I | Sbf I | 1000 bp |
| RAD2 | Prad2 #1 Apa I | Prad2 #2 Sbf I | Apa I | Sbf I | 1000 bp |
| RPS2 | Prps2 #1 Apa I | Prps2 #2 Sbf I | Apa I | Sbf I | 1000 bp |
| RPS31 | Prps31 #1 Apa I | Prps31 #2 Sbf I | Apa I | Sbf I | 1000 bp |
| SSA1 | Pssa1_2 #1 Apa I | Pssa1_2 #2 Sbf I | Apa I | Sbf I | 1000 bp |
| THI3 | Pthi1 #1 Apa I | Pthi1 #2 Sbf I | Apa I | Sbf I | 1000 bp |
| TPI1 | Ptpi #2 Apa I | Ptpi #2 Sbf I | Apa I | Sbf I | 1000 bp |
| UBI4 | Pubi4 #1 Apa I | Pubi4 #2 Sbf I | Apa I | Sbf I | 1000 bp |
| ENO1 | Peno #1 Apa I | Peno #2 Sbf I | Apa I | Sbf I | 1000 bp |
| RPS7A | Prsp7 #1 Apa I | Prsp7 #2 Sbf I | Apa I | Sbf I | 1000 bp |
| RPL1 | Prpl1 #1 Apa I | Prpl1 #2 Sbf I | Apa I | Sbf I | 1000 bp |
| TKL1 | Ptkl #1 Apa I | Ptkl #2 Sbf I | Apa I | Sbf I | 1000 bp |
| PIS1 | Ppis #1 Apa I | Ppis #2 Sbf I | Apa I | Sbf I | 1000 bp |
| FET3 | Pfet3 ′1 Apa I | Pfet3 #2 Sbf I | Apa I | Sbf I | 1000 bp |
| FTR1 | Pftr1 #1 Apa | Pftr1 #2 Sbf I | Apa I | Sbf I | 1000 bp |
| NMT1 | Pnmt1 #1 Apa I | Pnmt1 #2 Sbf I | Apa I | Sbf I | 1000 bp |
| PHO8 | Ppho8 #1 Apa I | Ppho8 #2 Sbf I | Apa I | Sbf I | 1000 bp |
| FET3pre | Pfet3pre #1 Apa I | Pfet3pre #2 Aar I | Apa I | Aar I | 1000 bp |

TABLE 7
PCR primers used for amplification of the
promoter sequences from P. pastoris
(SEQ ID NO 75 to SEQ ID NO 124)

Paox #1 Apa I (SEQ ID NO 75):
5′-AACCGGGCCCTCTAACATCCAAAGACGAAAGG-3′
Paox #2 Sbf I (SEQ ID NO 76):
5′-CATGGCCTGCAGGTGTCGTTTCGAATAATTAGTTGT-3′
Pgap #1 Apa I (SEQ ID NO 77):
5′-AACCGGGCCCAGATCTTTTTTGTAGAAATGT-3′
Pgap #2 Sbf I (SEQ ID NO 78):
5′-CATGGCCTGCAGGTGATAGTTGTTCAATTGATTGAAATA
GGGACAAAT-3′
Pgnd1 #1 Apa I (SEQ ID NO 79):
5′-TATCGGGCCCTATGGTAGAATCATCAATTGGAAT-3′
Pgnd1 #2 Sbf I (SEQ ID NO 80):
5′-CATGGCCTGCAGGTGATTTGTATCAGTCTTGTTTCTTTTC
TTT-3′
Pgpm1 #1 Apa I (SEQ ID NO 81):
5′-TATTGGGCCCGAAAGAAGGTTTATCTGACTGTTGCGCAC-3′
Pgpm1 #2 Sbf I (SEQ ID NO 82):
5′-CATGGCCTGCAGGTGTGTTTGTTTGTGTAATTGAAAGTT-3′
PHSP90 #1 Apa I (SEQ ID NO 83):
5′-GACTGGGCCCTTCAAGATCTTTTGAGGACTAGAGA-3′
PHSP90 #2 Sbf I (SEQ ID NO 84):
5′-CATGGCCTGCAGGTGATTGATATTTTTCCAAAATTAAAAAGTTAA-3′
Pkar2 #1 Apa I (SEQ ID NO 85):
5′-ATCAGGGCCCACTATCAAAGCTATCAATTGTGGAAATGGACAGCA-3′
Pkar2 #2 Apa I (SEQ ID NO 86):
5′-CATGGCCTGCAGGTGTCTTGAGTGTTGGAATTGAAATTAAGGA
AGAAG-3′
Pmcm1 #1 Apa I (SEQ ID NO 87):
5′-GTACGGGCCCACAGCTTTGGCTTGAACAAT-3′
Pmcm1 #2 Sbf I (SEQ ID NO 88):
5′-CATGGCCTGCAGGTGGCTAAATGAATGCGGGTTAGTGTTTGA-3′
Ppet9 #1 Apa I (SEQ ID NO 89):
5′-AGTACGGGCCCTAGAAAATTCACCACTGTCGGAAAGT-3′
Ppet9 #2 Sfi I (SEQ ID NO 90):
5′-CATGGCCTGCAGGTGGAAGTCGACGAAGAAGTTAGACTTGTTGTT-3′
Prad2 #1 Apa I (SEQ ID NO 91):
5′-GTAAGGGCCCGTATAGTTTGCAGACATAGTAGGAGAGTTT-3′
Prad2 #2 Sbf I (SEQ ID NO 92):
5′-CATTGCCTGCAGGTGATCCTTAGCCCAACCTGATGGAAAAACGG-
3′
Prps2 #1 Apa I (SEQ ID NO 93):
5′-GTACGGGCCCTCCTGAGAACGGACAGCAGC-3′
Prps2 #2 Sbf I (SEQ ID NO 94):
5′-CATGGCCTGCAGGTGATTAACTACACTGAAAAAGTCGGAATGTAC-3′
Prps31 #1 Apa I (SEQ ID NO 95):
5′-GTACGGGCCCTTGTTTATAGCCTATAATCGCAGA-3′
Prps31 #2 Sbf I (SEQ ID NO 96):
5′-CATGGCCTGCAGGTGTTTGGCTTCGTCGGCAATACGTGAATGCTT-3′
Pssa1_2 #1 Apa I (SEQ ID NO 97):
5′-GTAAGGGCCCGTTGTATCCATTCACTATTT-3′
Pssa1_2 #2 Sbf I (SEQ ID NO 98):
5′-CATGGCCTGCAGGTGAATGTTTAACTTTGTTTAATTTCTATGC-
3′
Pthi1 #1 Apa I (SEQ ID NO 99):
5′-GTAAGGGCCCATCTTTTCAGCTTCATCGTCAG-3′
Pthi1 #2 Sbf I (SEQ ID NO 100):
5′-CATGGCCTGCAGGTGGATGATTTATTGAAGTTTCCAAAGTTG-3′
Ptpi #2 Apa I (SEQ ID NO 101):
5′-GTAAGGGCCCTTCAACGAGACACTCTTCCGTCA-3′
Ptpi #2 Sbf I (SEQ ID NO 102):
5′-CATGGCCTGCAGGTGTGTGTTTGTGATAGATCTTGTATAT-3′
Pubi4 #1 Apa I (SEQ ID NO 103):
5′-AGAAGGGCCCAGAAGATTACCATAAATTGAGA-3′
Pubi4 #2 Sbf I (SEQ ID NO 104):
5′-CATGGCCTGCAGGTGAAAGCGACAAACGTCACGTGAACAAAAG-
3′
Peno #1 Apa I (SEQ ID NO 105):
5′-TATCGGGCCCAAAGAGTGAGAGGAAAGTACCT-3′
Peno #2 Sbf I (SEQ ID NO 106):
5′-CATGGCCTGCAGGTGTTTTAGATGTAGATTGTTATAATTGTGT-3′
Prsp7 #1 Apa I (SEQ ID NO 107):
5′-TATCGGGCCCTTTCATCCAGCTCTTTAACCTTAT-3′
Prsp7 #2 Sbf I (SEQ ID NO 108):
5′-CATGGCCTGCAGGTGCTTGTGATACTGCTGTTACCGTGTGAGTTT-3′
Prpl1 #1 Apa I (SEQ ID NO 109):
5′-TATCGGGCCCATAAGTCCTAGAACACCACTTGTTAGTAAAACCG
GT-3′
Prpl1 #2 Sbf I (SEQ ID NO 110):
5′-CATGGCCTGCAGGTGTTTCTATTAATTCGTCTCCCTAGCAAA
AAG-3′
Ptkl #1 Apa I (SEQ ID NO 111):
5′-TTTAGGGCCCGATATCGATTCCACTGCTCAGAGTCTTTTC-3′
Ptkl #2 Sbf I (SEQ ID NO 112):
5′-CATGGCCTGCAGGTGTGTGTAGAGTGGATGTAGAATACAAGTC-
3′
Ppis #1 Apa I (SEQ ID NO 113):
5′-AACCGGGCCCTTTTTCCTCTTCGTTGTGTGGTAAACTCGG-3′
Ppis #2 Sbf I (SEQ ID NO 114):
5′-TGATGCCTGCAGGTGGACTATCTAGAGACAAGTAAATTTCC
ATGTT-3′
Pfet3 #1 Apa I (SEQ ID NO 115):
5′-AACCGGGCCCTTTCGTACCAAATGGAAAAATCACGTACAA-3′
Pfet3 #2 Sbf I (SEQ ID NO 116):
5′-TAATGCCTGCAGGTGAAAACTAGATCCTCTTTGGAACAGGCCGT-
3′
Pftr1 #1 Apa (SEQ ID NO 117):
5′-AACCGGGCCCTCGAGTAACACACTACTAACTTTTTA-3′
Pftr1 #2 Sbf I (SEQ ID NO 118):
5′-TAATGCCTGCAGGTGTTTGAAAAGAACTACAACGACCACTGA-3′
Pnmt1 #1 Apa I (SEQ ID NO 119):
5′-AACCGGGCCCTAACATGATATCATGATGTACGTACAAACTAGGA
TCT-3′
Pnmt1 #2 Sbf I (SEQ ID NO 120):
5′-TAATGCCTGCAGGTGGATTGGTGATTTTGATGGTCA-3′
Ppho8 #1 Apa I (SEQ ID NO 121):
5′-ATTAGGGCCCGGTATAAGTATAGCACATGTTGACG-3′
Ppho8 #2 Sbf I (SEQ ID NO 122):
5′-TAATGCCTGCAGGTGTGCTTTGAAATTGAAGGGGAGAGGACGCTA-3′
Pfet3pre #1 Apa I (SEQ ID NO 123):
5′-AGCAGGGCCCTTGTGGTCCTATGAATTAACCATTTAA-3′
Pfet3pre #2 Aar I (SEQ ID NO 124):
5′-CTAGTCATGGCCTGCAGGTGTCGATGGAGTGTTGGCGGCAG
TGGTTAC-3′

b) Analysis of Promoter Activity in P. pastoris:

To test the properties and the activities of the different promoters, the 25 vectors prepared in step a) were digested with AscI and used for transforming P. pastoris via electroporation (using a standard transformation protocol for P. pastoris). Transformed P. pastoris cells were grown on YPD-medium (1% yeast extract, 2% peptone, 2% glucose) containing 100 mg/l zeocin. From each transformation 10 single colonies were picked on a YPD-zeocin agar plate and used to inoculate a 10 ml liquid culture. The eGFP expression was measured either when the cells were cultured on glucose as the single carbon source or on glycerol/methanol as the single carbon source. The amount of recombinant eGFP was quantified using flow cytometer analysis and the relative eGFP expression levels were calculated as shown below.

A untransformed P. pastoris wild type strain and P. pastoris transformed with a pPuzzle_zeoR_P_{AOX—}IacZ_AOXTT vector were used as negative controls for eGFP expression.

Calculation of Relative eGFP Expression Levels:

FL1 (fluorescence channel 1): GeoMean of 10000 events

FSC (forward scatter): GeoMean of 10000 events

$$\begin{matrix}rfu = \frac{1}{n} * {\sum }_{n}^{1} \frac{FL {1}_{sample} - FL {1}_{blank}}{\sqrt{{FSC}^{3}}} & rel \cdot Exp [ \% ] = \frac{{rfu}_{sample}}{{rfu}_{GAP}}\end{matrix} * 100$$

rfu: relative fluorescent units

rel.Exp[%]: relative eGFP expression normalized on GAP promoter

eGFP Expression on Glucose as Single Carbon Source:

Shake flask cultures in 100 ml Erlenmeyer flasks on 10 ml medium (containing 1% yeast extract, 2% peptone, 100 mM potassium phosphate buffer pH 6.0, 1.34% yeast nitrogen base with ammonium sulfate, 4×10⁻⁵% biotin, 2% glucose) were inoculated with a single colony from a YPD-zeocin agar master-plate and cultivated at 28° C. and 180 rpm. Glucose was added to a final concentration of 0.5% every 12 h. Samples were taken 16 h, 40 h and 67 h after inoculation, diluted with sterile PBS to OD₆₀₀ of approximately 0.1-0.2 and analysed on GFP expression by flow cytometer analysis (BD Facs Calibur). The results are shown in Table 8.

eGFP Expression on Glycerol/Methanol as Single Carbon Source:

Shake flask cultures in 100 ml Erlenmeyer flasks on 10 ml YPG-medium (containing 1% yeast extract, 2% peptone, 1% glycerol) were inoculated with a single colony from a YPD-zeocin agar master-plate and cultivated at 28° C. and 180 rpm. After 22 h cells were harvested by centrifugation (1500×g 5 min.) and resuspended in 10 ml MM-medium (100 mM potassium phosphate buffer pH 6.0, 1.34% yeast nitrogen base with ammonium sulfate, 4×10⁻⁵% biotin, 0.5% methanol). Every 12 h methanol was added to a final concentration of 0.5%. Samples were taken 22 h, 42 h, 64 h and 90 h after inoculation, diluted with sterile PBS to OD₆₀₀of approximately 0.1-0.2 and analysed on GFP expression by flow cytometer analysis. The results are shown in Table 8.

TABLE 8
Relative eGFP expression levels in % (standardized on eGFP
expression under the GAP promoter) in P. pastoris.

| | 16 h | 40 h | 67 h | 22 h | | 20 h | 44 h | 70 h |
|---|---|---|---|---|---|---|---|---|
| | glucose | glucose | glucose | glycerol | => | methanol | methanol | methanol |
| AOX | 3.6 | | | 1.0 | | 119.0 | 162.9 | 184.9 |
| GAP | 100.0 | 100.0 | 100.0 | 100.0 | | 100.0 | 100.0 | 100.0 |
| GND1 | 0.0 | 0.0 | 0.0 | 0.0 | | 4.3 | 8.4 | 66.7 |
| GPM1 | 41.0 | 25.2 | 24.8 | 23.5 | | 19.2 | 22.1 | 25.2 |
| KAR2 | 134.3 | 12.3 | 10.9 | 71.7 | | 37.6 | 35.6 | 42.9 |
| TKL1 | 1.4 | 0.0 | 0.0 | 5.4 | | 4.3 | 7.1 | 9.3 |
| PET9 | 1698.7 | 483.7 | 490.3 | 1203.9 | | 740.6 | 750.6 | 926.2 |
| HSP90 | 81.1 | 6.4 | 5.4 | 39.5 | | 21.2 | 21.6 | 32.9 |
| RPS2 | 0.0 | 0.0 | 0.0 | 9.4 | | 5.7 | 7.9 | 12.4 |
| SSA1 | 0.0 | 17.3 | 21.3 | 11.4 | | 9.1 | 9.2 | 30.1 |
| PIS1 | 0.0 | 0.0 | 0.0 | 3.0 | | 0.0 | 2.8 | 6.5 |
| FET3 | 0.0 | 0.0 | 0.0 | 3.9 | | 0.0 | 3.0 | 7.0 |
| FET3pre | 0.0 | 0.0 | 0.0 | 3.9 | | 2.2 | 2.3 | 7.1 |
| RPS31 | 6.3 | 1.0 | 0.0 | 2.9 | | 2.9 | 4.0 | 7.7 |
| ENO1 | 22.3 | 46.6 | 45.8 | 30.6 | | 28.9 | 17.3 | 26.1 |
| PHO8 | 0.0 | 0.0 | 0.0 | 0.0 | | 2.3 | 1.9 | 6.1 |
| FTR1 | 0.0 | 0.0 | 0.0 | 0.0 | | 0.0 | 1.9 | 5.6 |
| NMT1 | 0.0 | 0.0 | 0.0 | 0.0 | | 0.0 | 2.3 | 5.0 |
| RAD2 | 0.0 | 0.0 | 0.0 | 0.0 | | 1.4 | 0.0 | 5.4 |
| RPS7A | 17.7 | 2.7 | 1.4 | 13.7 | | 10.0 | 6.6 | 12.4 |
| MCM1 | 0.0 | 0.0 | 0.0 | 1.4 | | 2.9 | 0.0 | 6.0 |
| UBI4 | 0.0 | 0.0 | 0.0 | 0.0 | | 1.2 | 0.0 | 4.2 |
| RPL1A | 0.0 | 0.0 | 8.7 | 9.4 | | 4.9 | 2.1 | 10.9 |
| THI3 | 0.0 | 0.0 | 1.2 | 1.2 | | 13.7 | 13.4 | 41.5 |
| TPI1 | 0.0 | 12.1 | 13.9 | 4.8 | | 57.3 | 38.0 | 91.7 |

From Table 8 can be seen that there are promoters with different transcription levels on different carbon sources in a range from 0% to 1600% available (relative to the eGFP expression under the well known GAP promoter, which was set as 100%). Real unexpected were the high eGFP expression levels obtained from the vector pPuzzle_zeoR_P_{PET9—}eGFP_AOXTT (see FIG. 2), wherein the eGFP is under the control of the PET9 promoter, i.e. a 1000 bp fragment from the 5′-non coding region of the PET9 gene. The eGFP expression obtained from the vector pPuzzle_zeoR_P_{PET9—}eGFP_AOXTT under the experimental conditions as described above ranges from about 480% to about 1700% when the P. pastoris cells were cultured on glucose as the single carbon source and ranges from about 740% to about 1200% when the P. pastoris cells were cultured on glycerol/methanol as the single carbon source (standardized on eGFP expression under the GAP promoter). The nucleotide sequence of the 1000 bp fragment from the 5′-non coding region of the PET9 gene of P. pastoris is shown in Table 9 below.

TABLE 9
Nucleotide sequence of the PET9 promoter (a 1000
bp fragment from the 5′-non coding region of the
PET9 gene) of P. pastoris
(SEQ ID NO 125)

CAGGTAGAAAATTCACCACTGTCGGAAAGTTGTCTACTTCCGTCGGTTGG
AAATACGAGTCTGTTGTTGAGAAGTTGGAGGAGAAGAGAAAGGCTGAGGA
AGCTGAGTACCAGGAGAAGAAGAGAGCTTACACCCAGAGATTAGACGCAG
CTAGTGCCGAGTTTGCCCAAACCGAGGAGGGAAAGCAGTTGGCTGCCTTT
GGTTACTAAATAGTAAAGTAGGGTATCTTCAAGTAATAGTATACTAACCA
TCTGAAATAACCACCGTCCTGTAGTTTTTTTTCGATATCGAAGAGCCTAT
GCTAGTACTGTGGATTTGCGCTCCATCCAACATCTGTGCGCAAACTAAAA
CTTCCGAGACTGACATCTACCATCGCTAGACCCTAAGTAAAACCAATCTC
GCGTCCGAACTTTTAAATTTCAGTCCTTAAAACTTCAGAGCATTGGTTGT
AGTTTCCGGATCTGAGGGGTCGTATTGGAGTCAAGAGACGGAGCTGCCTC
CACAGCGCGAAACGTCAACCCCAACACCAACCTGAATTTGCAATCACCAT
GGGGACAAGTTTCAGCAGTCAATGGGCAATTCAGACGTTGATACGGTACC
CATTTGCTAAGCTCAATGACGATCCATCCAACTTCAGAGAAAGGCCTTTC
TCTGGTATGCTCTGGTATTCATTCGTCTTTTATCACTCTCGTTGCACAAT
GCCCGGGTACTCCCGGAACAAGGGAGTCTTCCAGCCAAGCTGTACAGAGT
GAAAAATAGAAATACACCTTTGCAATCAAGACGCGCGTTGGCCAATCACA
AGACTTAATCGGTGCAAAGAAGGATTACCAAATTTTTTTTTCCCAAAATC
GCTATATAGAAATAATGGAGGAAAAAGGGTTAATATAAAGGAGAATTCCC
CCGTTTTTCTCCCCTTGTCTTTTCTTCTTCAGGCTTTCTTACAAATCTAT
AATATTCCAAAATGGCTGACAACAACAAGTCTAACTTCTTCGTCGACTTC
ATG

Additional interesting promoter sequences with different transcription levels on different carbon sources in a range from 0% to 135% (relative to the eGFP expression under the GAP promoter) are shown in Table 10.

```
TABLE 10
Nucleotide sequences of promoter sequences
(1000 bp fragments from the 5′-non coding
  region of the respective genes) of several
  P. pastoris genes
  (SEQ ID NO 126 to SEQ ID NO 147)
and respective ERGO ™ database information
```

GND1 (SEQ ID NO 126); RPPA07075 - Pichia
pastoris (IG-66), Contig1891_9813_11291
TATGGTAGAATCATCAATTGGAATGACCCTATCGTTGCTATACAGA
CTCGTAGCCCCATTTCTTGTTTGTTGGTTTTCGTCGTCAATTGCCT
CCAAAATTGAAGAGATGGCACTTCGTCGCGTGGGTTCCTGGGAAAT
ATTTTGAAGTGGAACATCGTTGAAATAAGGCAGTATATCATGCTGA
TTCTCAGCATTGGAGTCAGTAGCTTCAAGTGAATCTATACTGTATG
AGTCGGAAGAGGATTTCCGGCTCGTTTTTGTCTTCATTTTGTTATT
AGAAGGAATGATAACGTTGGGAAACCGGAGGTTGGAGATTTTGTAT
ATATAAAACTTTCTTGGAGCTTATTAATAAATGCGGGATGCAGTAA
ACTTGCATATATCTATTGTAACACTTTTGCAATAGCTGCATGCCTT
GACTCATCATTCAGTATCGTGTGAAAACCAATGATACATCCGTACA
TTCAAACTACAACCTTCCTCATTAGTAATTCTTTTTGAATTTTTCG
GAACCCGAAGCTCCGCCTATCCCCCCAACTAACACATCTTCCAATT
TGGGTGGGAGAACACCTAGCAACATCACGATCATTGCGCGAACGTT
CGCACTGTATTTTTTTCTCCCAAACACCCAACTTCTAGGCCAAATA
TCCACTTCTCGGGGTTCTATTCACCCATTTAATTGTTGGCCTTAAA
AGTCAATTGAGTTCCAATCATAGTCCCTAGTTGATTGCTTGTAGCA
AATGCCACAACAGTAGGCATTTACGTCCTCACAGTCTCTTCCCTTG
TCCCTCATTGATACCTCTTTATTCTCCCCCACCACCATACACTACC
TTCCTCGCACCCCTGTCATCACAACCGCAATATAATCGATGCGCGG
TTTCTTGCCTAATCCATCGTCCAACAGAGAGGTCGCTCTCCTTATA
TATATAGTTGATCCCCCTTTTTTTCTACCCTTGCAATTTTTTTTTT
GGGACCAAAGAAAAGAAACAAGACTGATACAAATATG
GPM1 (SEQ ID NO 127); RPPA05288 - Pichia
pastoris (IG-66), Contig1212_2407_3064
GAAAGAAGGTTTATCTGACTGTTGCGCACCACCGAAACCAAAAGCG
GGTTTAGCTGCAGAACTTTCAGTGGCCGGCTTGTTGCCCAAAGAGA
CTGTTACTGCCTCCATCGGTCTTGGTAGCGCCTGAACCAAACGAAA
AAGCACCACTGGGATTATTGTTTCCTCCAAAAGACGCGGTCGTGTT
TGATGCTGTTGAGGCCGCAGGCGCTTGACCAAAACTAAACGCTTTT
GATGTATTGTTTGTAGGGGCTGTTGCGTTGTTAGAAGCTGGCTGGT
TGTTCCCAAAGGAAAAAGCACCTGTTGATCCAGTGTTAGATCCACC
TGCGGCATTCCCTTGAGCTGCGGAGCCAAAAATTGATCCTTGGTTT
GAGCTCTGTGTACCAAATAAAGATGAATTGGCCTTAGCTCCGTCAG
CAGGAGCGTTTGCTTGTCCAAAGGGGCCTCCAGACGACGTTTGCGT
GTTGTTGGCATTACTAGTATTGGCAGGCTGATTTCCAAACAATGAG
GGCTTGGAGGCGTTCTGGGAGTTACCAAATGAGAACCCTGTATTTC
CTTGGCCAGAGCCTGAGAAACTGAACCCTGATCCAGACATTATCAA
TACCTTGGGTTATTAGTAGTGTCCGTTATTTTTCTGTTTAGGTTAC
GATTTTGCCAGATTTTTTGGGAGGAGGGAAACAAAAGAACCAGTGC
TACACGACCTTTAAGTGCCATCAGGCATCCTGTTTTCTCGACCTCA
TCTCATCACATCCGTCAGTCTGAGCTTTCAGTTCTCAGTTTTCGAT
TGACTCTTGCCCTGCTGCGCGCACACCATACCCTGGCTCCCTCTCA
TGCTTCTGGCGTTACCCCGGGAATCGTACATCCATGCCGCGAATCC
CGGACAGGACTCAGACGGATTTCACTATTCCTAGTGGGCACAACCT
CCATATATAAGGATACTTCTGCCCCCTAGCTTAAGTGCCTCTATTT
TGTCAGTAACAACTTTCAATTACACAAACAAACAATG
HSP90 (SEQ ID NO 128); RPPA05876 - Pichia
pastoris (IG-66), Contig1951_9775_11895
TTCAAGATCTTTTGAGGACTAGAGAGCAAGACCTGCATTTGATAGT
CGAAGAGGAGAGGCAATTTAAAGATCTAGTATTGGAGAAGGACAGA
CTGCTCGATGTTCAAGATAAGAATGCTTTGAATCTAAGGCTACAAC
TAGGTTTTGCTTCCCACAAGGTAATTGATCTAAACCAATTACTTCC
TGTGCAAAAAACACTTGGAGCCACTACTAGTCTGGAGTTAATCAAC
TTTCAAACAGACATCAAAACTTTACACGTCGTAAACAATGTCATGG
ACAAAATCCGGGTTAAGGTCACTGGGGAGAAGCGACTCGTGACACT
GACTCCTGACAGCACCAACAAGGCTCTAGAATTTCCCACTGAGAAT
GATAGGCCACTGCAAGAACAACAAAATACAGAGCATGAAAACGAAA
ACGGTGCTCAAGATCCCATAGACTCAAACCAAACGAATGATTCTCA
GACATACTCCACACCTAGCCGTATCCATTATACGACTAACGAGGAG
AGTTCCTCTAAGAAGGTTAAAATATCAAATTGAGCATGATACGATA
GCCCGCTGGCAAGGAATTAAGTGACACTAGATCGCAAATAATTCGG
AATCACTTTATGTGGAATCATCATTCTTGATAAATTGTGATAAAGT
ATCGTTCGCGAGATGGTTTTTCCAGAAATTTCTTGTGGATCAATAT
CTTTAAACGATAGTAAACGATGTTGACAGCTCCAACAATGTCGGAA
CTGCTCATCAAAGACGGACAATTTCCCTATTAGCAGAAAAATAGCC
CATTTATCATCACCGCAGCGCTATTACTGAATGATTTTTTTGAATT
TTTCCTTGTCTGTGAAGCCGCAAACCACATTTTCTGGCGCGGTTAC
CCGGTCATAGAAGCTGGTGGAATATTCCATGCTACCTCGACGATGA
GCTTGCGAAATATATTTAAGTGTAAGAACTCCGCGTCATTAAGACT
CTGGTTAACTTTTTAATTTTGGAAAAATATCAATATG
KAR2 (SEQ ID NO 129); RPPA06939 - Pichia
pastoris (IG-66), Contig1900_3677_1641
CCACTATCAAAGCTATCAATTGTGGAAATGGACAGCAAAACGGAAA
CGCAGTCAATGACGATGATTTCAGAGCTAAGCGATTGTGCGAAAGG
TGGTTGGACAATCTGTTTATGCTTCTTTATGAAGATCTGAGGGTGT
ATACGATGTATCGTGCCGAACACATGCACTTGACGGCACAGCAAAT
GGAATTCAAGAAGACCACTTTAGAATGGGAGTTAATAGGGATGGTT
TCATGGAGGTTAAAACACTTCAAGGAGGCATCTGAAGCATTCAAGT
ATGCACTAGGTCTGAGGTTTTCGGTCAAGGCATGCAAGAAATTAAT
TGAATTCTATCTGAACGAACGCTCCAGAATGAACCAGCCAGAAACC
TCAATTGCCCTCAACAACTTAAATCAATCCACATTATCCATCCAAG
AGATTCTCAAGTATCGTTCGTTCCTCGATATCAACCTAATTTCAAA
CTTGGTCAAACTAGGAGTTTGGAATCACCGCTGGTATGCTGAGTTT
TCTCCAAAACTCATAGAAAGCCTTGCGGTTGTTGTGGAGAACGGAG
GGCTTATCAAGGTAGAAAACGAGGTTAAGGCTACCTATTTCGATTC
ACAAGATGGAGTTTACGACTTGATGAACGAGGTATTCAAGTTCATG
AAGCATTACGATTATCCTGGGACTGACAACTAAGAGCTCCTAGTGA
AGACTTGAGATGGACATGATAAACAATTATAGTGAAAATAGAAACC
ATAATACAATATTCTAATAGAGGAACCGTTTACCTGTGGTTCCTAT
TGTGGCCTACTGTTACTAGCTAGTGTAATACACCCTTGCCTCAGCT
TTGCAAGTTGACAACTCAGCCAAATGATCTTTGAATGCGCGAAACC
TCAAGGTCCATCGAATTTTCTCGAATTTTCAGTGTTTTCATACAGC
GTGTCATCTTCTTTCGCGTACTTATTTAAATCGTACCCAGATCCCT
TCTTCTTCCTTAATTTCAATTCCAACACTCAAGAATG
MCM1 (SEQ ID NO 130); RPPA08348 - Pichia
pastoris (IG-66), Contig 1815_6838_7665
ACAGCTTTGGCTTGAACAATAGTGGTTGGATGTTACCCGGTGCGAC
AAGTGCTGAGTTGCGGTAATTTACGATTTCAGCGTCCACCAGAATG
GGAATTCCGGGTAACACGCATACCAGGGGAAGAATATCACAAAGAT
CACAGAGATTGGATAAAACTGGACCAGAAACTACCAAATAAGTAGA
CCGTCATACTCAGCTCCTACACCAAGAGGTACTTCAGCCTTTTACC
GGTTTAAAAAGCCCCCCGAAATCGACTAATTACCGAGGCATTATGT
TTACTACTGATGGAGATTTCGAAATATCCTTCCCGATTAGTCCAAC
ATCTCGAAATTAAACTGCGCAGCACTATGCCGAAAGCTATATAAAC
AATCATTTCCCCAACTGGAACATCTTTTTTCTCTTTTCTTCGTGTA
TCATCCTTTGGTCTTTTAATCTTTCAGAAAAGTTTTCATTAAAATG
CTTCCAGCTGGTGTTATTTTAGTCTTTTGTCTACTTTTCATTATCG
GGGTAATTATCGCAGTTATCCTGGGCATGAAGTGGTACAAGAAGAG
AAAGAACTGAGCATGGACGAAGAAAATTACTAGACCAAAGATGTAT
CACCAAACAAACCCCCCCAAGAATCTGTCATAAACGAAGTTGTTAT
AGGATGGTTATCACTCTCACATTTTAATACAGGAATACCCTAATTT
TTCCTCAGTTCGGTGACGAATGGAGTTGTAGTTATCTTTCTTTCCC
TCGTTTCTGCTCTTATTTCCCCCTCTCGTCGCATCATTTGCATCAA
ATTGGTGCAACGTGCGCGCACGCTCGGCTTTGGCTGCAACGACTTT
CTGTCGTGTACCGACCCATATTCTATCGCTTCGGGTAGTCCGCAAC
ACCCTCAACTTCCTAATTTTGTGTTCTTACAAGCTGCAAAAAAGTA
GGACTCACTCAATAAGGTAAGTCCCCAAACATCAATCCCAATTGGG
TAATCCATCAAACACTAACCCGCATTCATTTAGCATG
RAD2 (SEQ ID NO 131); RPPA09286 - Pichia
pastoris (IG-66), Contig1185_5060_8191
ATGGTATAGTTTGCAGACATAGTAGGAGAGTTTGCGGGCTTGCAAC
TCTAACAAGTCTCTGTAGTCGCTGAGACTAATGCAATCGTCAAGAC
TACTGGGCTCGTTATCCGAAAAAGTCCCGGAGTAACTAGTCGGTAA
CTCTCTGTACCGAATCATATTCATCCTGGGGTTGATCTGATCTTGG
TCCATGTCATCATTCTCTCCATTTCCCGTAGGGTAGGGCAATCCCA
CCAGCCACTTGTTAACCTTTTCTTTATGCGGCATATTGGGAGTCTG
CTTGGGGTTGAAGCTTTCTGGAAACTCCTTTTTCTTGATCAGATCG
CTGTCACTACAGAACCCAGGATGCTGCTGATAAGGGGAAGAAACAT
AGCCCATGCCCATTGAGTTGAAATTGAAGGGTTCCTGTGCTGCACC
TGCATTCAACGGATATACATGTATAGGATGATGGATCATGTGGCTG
TAAGAGGGGCCAGACGCTGAGTTATCACCATTAACATTGTTAGGAG
ATCCTGAGCCTTGAAAGGGCACATTGCTAGTCTTTCCCGTAGTCAT
TACAAACAGCGGGCCATAGTTCCCGTTGTCACAAGGATATGCAATT
CCTGTAGGGGTATAGATGTGTCTGTTGAATGGGGTCATGGCGGGAT
TCTAGAAAAGAGACGGCCCGTTAGTTAAATCCACCCACCGATTATT
GAGGTCCAACTTACAATTTGGTTTTGACGGTCTTCCATTGGAGGAT
ATGTTGTTCTGAGTTGGTGGTAGTTACGTTTATTTCTCCTTCCTTT
TTGGTTTGTGTTAGAGCAAGGACACGGAGCACTGACTACCTTCAAG
GCCATTTGTTAAGAATCCACAGATAGACACTACCACATGGGAAATG
TTGTTAGGGAAGAGCTGATGCTGAGGCTCAACATCGCGCGACTCCT
TTTAGTCAGTTTCCAAGTACGGTTCACTGTAGACATTTCTCTTTTT
AGTTTCCGTTTTTCCATCAGGTTGGGCTAAGGATATG
RPS2 (SEQ ID NO 132); RPPA04904 - Pichia
pastoris (IG-66), Contig1847_1328_537
CTCCTGAGAACGGACAGCAGCGCTGGAGGCGGCCTCTTTAACGGTG
GCGGCGAAGTCAACAAGGGTAGTTTTGATTTTGCCTAACAACTGTT
GCAGGTCCTTCTCAGTCTTTGGGTGGCTCTTCTTGAACTCGTCCGA
TGCATCCTTGACGACCTTCTCAGCGTCGTCGGCAATCTCGTCGACA
ACTTGCACTGATCCATGAGGGTCTTGGGTTTGTTCGACTTCTGGAA
CTGGTTTTGGCTTGCCAGCGGAAACTCCTTCTGGGGGTGTGGCAGC
TTCGGCGTAGGTACTATCAGTGTTAATATCGGGGATTGGTTGATGT
GCGAGACGAGGCAGGCGGGAGATCCGTCTGCCTCGTCCAACTGCGA
TAGCTCTTACTTACCTCATGATGCTTGTAAAAAAGTTAACTGAAAG
ATGGAAATGGGAGGGGGAAAAGAATTGTGGTCAAATCCACGTCTTG
CGATAACCTCATACATTTGCTGCATGATTGGGGGATCATCGCAATT
TCGGTCCTTGTGACACACGCGAGCTTTCCGCCTGGGGTTAGAGCGC
GGAGCAAGCATACTATCAAGCAAGAAAAAATGGTATGGAGAAACCT
AATTGGTTAAGATATATGAAAACTACGACGGCTTCATACGTGGTAT
CTCTGGTTGAGCTACTACGAACCATTTTCCCCCTTCAAACCCTTTG
GCCCATGCCATTCATGCCTTGCCTCTCTCTCAAGCAACTAAGCAAT
CAAGCAATTTCCCGCCTTGCTGCACATGACTGTTCGGAAATCGGAG
ACCCAAACACCACTTGTTATCTATGCACGTGATTTTTATCCAGGGC
AATAAATACTCACTTTTGCTTCAAAACGCTTGGGGCGCGCGAGCGG
CAGGCTGGGAAAAAAATAATCTCAGACTTTTCAAAAGACTCTCCTC
TTTAATCATTGAATACCGTCGATCGAAAACCACCACCATCGGCTTT
CCACGTACATTCCGACTTTTTCAGTGTAGTTAATATG
RPS31 (SEQ ID NO 133); RPPA07467 - Pichia
pastoris (IG-66), Contig1605_1365_1817
TTGTTTATAGCCTATAATCGCAGACTTTGTTAACCCGTAGGAATAA
TCCTACCCACTAGAATTAGATCTTCCAACCATACATAGCGTGTACT
ACAACTTAAGCTGGTCCTCTTCTTTTATCATTCGCGCGGCTCTATC
TCATCTACACCTCCATTGAACTAATGGAAAGCAAATACGCCGAAAA
ATATCAAGATCGATTGATTCTTGGAGATGATGGCGATGAGGACGAA
TTGTTTGACGAGCTGGAAAAAGATATTGAGGATCAATTCTTGGCCA
AATACAGAGCAGAGAGAATCCAACAGTTAAAACAGGAGATTACCAA
GATCAAGGACCATAGTTCAAACATCAACCTCAATGACCACGGTAAC
ATGAAAACAATAGATACTGATGACGAACTATTGAAAGAAACTGTTG
ATAGCGAACGTGTTGTGATCCATTTCTTTAACCCATCGTTTAGCAC
TTGCCGTATCATGGATGAGAAGCTGTCTATAATCAGCACCAAACAT
ATTGGAACGCGTTTTTTCAGAATTGAAGCACATAGAGCTCCATTTT
TAGTTGCAAAGCTTGGTATCAAAGTGCTTCCATGTGTTGTATTGTA
CTACAAAGGATTAGAAAGGGATAGAATTGTCGGATTTGACAGATTA
AGTAATTCTCAGACCAATTTTGAGCTAGAAGCTTTAGAGGAGTTAC
TCTTAGATAGTGGAATTGTGGAACGAAGAACTGTCGATTTTAGCAA
CCTGAGAAACAAGGTCCAAAACAAGGTGGATCAGTCAAAATCAGAC
TCAGAAAGTGATCTAGATATGTGATAGATGGCGGATGGCAGGTTCA
TTCTAGTGTTTCACGTGACACACGTGAGCGTTTAAGGGCACACACC
CTGACTGACGCGCGAACATCTAATCTGTTCCGCATGAAAAAAAAAA
ACTACCTCGACGAAATTCTCTTCTAGACAGTTTTTACATTGGTAAG
AAAGAAGCATTCACGTATTGCCGACGAAGCCAAAATG
SSA1 (SEQ ID NO 134); RPPA10651 - Pichia
pastoris (IG-66), Contig2026_5140_3168
GGGTTGTATCCATTCACTATTTACTCTTTGTTTCATTTCTTGAATT
ATTTGGATACTACTCTGCTGGCAACTCTACCAGTCTCAAACGCAGA
CCAGGTTCGCAATTTGATTAGAATGTTCGTGAGCTCTTACAATGAA
AAGTCCATGTACCTTGCGGCTAGTTGTGAATTATTTTTAGTTCCTT
CTTTGTTGCTATCCTCTTTGAAGTCGATTATATTGCTGGAATGGTA
TAGGGCTCCCTTTTCATTTATCAGGCAATTAATCGTGGTATTCTCC
GTGATCTCGTTTCTGAGATTAAGATATCAACAGAATGTTTACATGA
AACAATTAGTTGATAGTTATGATTTGAAGATCAGTCAACTCTTATA
CCATCCCCAACTTCCTCAAGGATTCAGGTTGGGATATTTACGATTT
AAGAGTCTATTAACAAGCACGCTAGGATACTTAGAATTGGAAAAAA
AGACCAGATAATGAGATTGAACTCGAAATTTAGGATCACCCATATG
ACGAAGAATTCATTTAGATTATTGAAGGTGTTTTCATGTTTACCTC
CATGAGACCATTTCTGTCACAGCAAATACAGGCAACGCTTTTCACC
AGAGCTTGTTGGTACAACTTTTCAGATGACGCCAAATTCTCACGCG
CCTCACTTTGTGCGGCGCTAACAATAGGCCATTTTTTTGTACCTCC
CGGATGGTTCAGCTCAATCACTCGATTGAGAGGTTTTTGTTCCGCG
ATTTTTGTTCACCCCACACTTTTCTCGAAGGTTCTAGCAATCAAGA
TAAACACCGCAAAGAGAGCCGCAGGAACCATATGTGGTACCACAAG
TGGTCTTAAACAACTCTGGTAGAATTCGATGGAATTCGATGGAAGC
CGATCGACTCCGATCGAATTGAAGCAATTCGTATATATAAGGAGAA
CCTAGTTCCACCCCTTACTCGACCATTAGTTTACAAGACTAACTTC
ACAGAAGCATAGAAATTAAACAAAGTTAAACATTATG
THI3 (SEQ ID NO 135); RPPA07820 - Pichia
pastoris (IG-66), Contig2116_14564_15586
ATCTTTTCAGCTTCATCGTCAGTGATATTTCTCAGCCCACAGACCA
AGTCAACTTTGGAATCTAACAACCTTGTTCTTACAATGTTAGAACT
CTTAAGTCGCATGCCATGATCTTCAAGCTGAATTTTGTGAAGGAGG
TCAAACCCCACAATGGCATCTAGTTGTTTAGAATACATGCCTTCGA
CAAGTGTTTGAGTGTCCAAAATCAAGAGCTCAAAATTATTGAATTT
GTCTGCCAATAACGCCGTAAATTGATTAGTGTCCAGCCCACCAACA
ATAGGAGCACCTATAGTTAATTTTTCAGATAAATTTAAGTTATCAA
GGTAAAGGAGCTCTAAGTTTACCCCTTCCAACAGGGTTATTTGAGA
ACTCAATAAATTGTTGAATTCAAAACCAATTGTCTTTGAATTCTCC
ACTGGAGCTTCCTTGCTGAAATTGATTTTGATACCATTGGCATCAA
AGAGACCCGTATGATAACTCCATAAAAAGGGGAGATGATAGGCCTT
AAATTCATCGTTAATCTGCAAATTTATTCCTGACATGTCTTTGTAA
ATAGTTATAGTTCAGAAACTGGAATTGAGCTCAAAAAACTGGAATC
GAGCGGATATTTGAAGATTGATGCCTTACTCATGAATTGATTGATA
AGAGCTCCGTGATTCACTCTGTCAATGATTACCCCTCTCCTACCCG
ATTTGGGACTTTTTCTTCAGTCTTGGGGACTTTTTTTCATATGACT
TGACCTTGCTTTCCCAATAGGGAAGGACTCACCCATGGATGATTAA
TCATACCATCATGAAATAGGGTTAGGCTGTAAATGCCTCAAAAATG
GCTCTTGAGGCTGGATTTTTGGGTATTGGAATGTTGGTAGCAATTG
GTATAAAAGGCCATTTGTATTTCACTTTTTTGTCCTTCATACTTTA
CTCTTCTCAACTTTGGAAACTTCAATAAATCATCATG
TPI1 (SEQ ID NO 136); RPPA06839 - Pichia
pastoris (IG-66), Contig1564_2883_2026
TTCAACGAGACACTCTTCCGTCAGTTCCAAAACCATAAGTTTGCCG
ATGTGTTGGTCCTTGTAACGCATGGAATTTGGGCCAGGGTATTTTT
GATGAAATGGTTCAGATGGTCTGTGGAGGAGTTTGAAGGCTTACGA
AATATACCACATTGCCAGTTTATACAGATGGTTAAGGGTGAAAATC
AACGTTACACCTTGACGACCCCATTATTACGATGGCGTGAAGGAGA
TGAAGACCGGGTAGAAGAAATAAGAAAAGCGGTACAGTTTAGGTCC
GGAGATCTAGGGAAGGAGGCCTTAGCTTATATTGTAGCTGCTGAGA
GAGAGGCAGCTGCTGGAAGATCTGAAGGCCCTATCACGTATGATGA
TGGTGATGACCATTAGAGAACGCCCAGAGATTGATAGCCAGTTCTT
GGACAACAATTCGGAACTTTATTCACGGTGCAAACATGATTTGTGT
GGATAGCTTCAAGTCAGACATTTCATCTCATCCCCCCTTTTACTGC
TGCTAATCACCGTTAGTCCGACAGTTACTCTAATCAATATTTATTA
GTGTTTTAGTTGCGCAAAACTCGAGCCTCTTTTCCTTATCTCTTGA
CACTTCCTGGAGTCGAAGTTTTTCAGCGCAAATTCACTCTACAATG
TCTACCGATACTAGACCGCCTATCTTCCCCCTCTAAATAGCCTATT
GGAAGGGTGCAATAAGGTATATAAATCTGGCGCGATTCCCCCGGAC
TTTTATGATCCACATCACCTCATCTTACTGCCCTCACTCTCTTTCC
TGATCCTCCCAGGTCCACCGATTTCCTCACTATCGTCGGATTTCTC
CTTCCAGCGCCCTAGAGAATTCCGTAACCACCGCAAAAATAGCAGC
CCCCCCCTCACCCATTTTTTTATTTAAAAGAACACCTTACTGGCCC
GTTTTCGTTTCTCCTTTACTACAATTGATTTTTAATTTTCAGTTTT
TTTTCATTGATATACAAGATCTATCACAAACACAATG
UBI4 (SEQ ID NO 137); RPPA07243 - Pichia
pastoris (IG-66), Contig1945_6169_6092,
Contig1945_6013_4858
AGAAGATTACCATAAATTGAGAACTCAAATGACCAAGCCAGGAGTC
AAGAAGAATTCTCAAAAGGGATACGCAAATATTGATTCGGATGACG
AAGAGCTGACTGAGGCTCTCGATTTCAAATTGGCACCAAGGTCAAC
AAAAAGTCTAGACATAACACGGTCTGGACTACCGCAACAATATAAT
TTTGAAAATGTTCTTAGTAGCGACGAAGAAGATGGAGGCGGAGAGT
TTCAGGGTGATTCTGTATCCCCAATTAAAAACAGTTTTCATTCAAG
TCCAAGGAAGCAGCGATCTCTACTCCACGTCATAGACGAAGGAAGT
CCATCTATTGGCCCAATTTCTGTTCAACTGAAAAGAAGTTTGACTT
CTCCCACAAAGAGACACAAGAATGCTCAGGAAGTGATAAGCCAACC
AGAAAGCATGACATCCTCAAACTTGTTTTCGCCTACACATTCCTCC
GCCTCAACTACATCAATAATATCTCCCAAACCCCAAGCGGCAATGA
TCCCAAGTCTTAACCCAAATAACCCTTTTTATACTGATACTAATAG
CTAATATGTAATAATGATTAATAAATGCACCACTTTAATTTCTTTA
TCAAGAATAGTTTTACTATTTCTCTTTCGGGCATATACTTGACCCG
CTCCATATATTTCACCTTTATCGTGACATTATCTGCTGCACGACCC
GGAATTACTTTAAACTTCTAAGAATTTCCAAAATAGGAATCGGGAT
GCCGTATTTTCCAGTCTGCTGGACAGAACTTGTGCTCTCTAAAATG
AATTGTAGTGGTCCTCAAAGGCTACTGCTACTCACAGTCTTTACTA
CTCCAGATTGATCCATTCCTTGGCCCATGCACAGTAAATTAGACTA
CTGATCTGATGTTGTATACTTTTGAGCTGATTGATTACAGTTAACC
TGGTCTTATCTAATCTCTCGTTTCTTTTTATCTCTATTCCCATTTC
TGTACTCTTTTGTTCACGTGACGTTTGTCGCTTTATG
ENO1 (SEQ ID NO 138); RPPA05175 - Pichia
pastoris (IG-66), Contig1903_1777_3087
ATGAAAGAGTGAGAGGAAAGTACCTGGGCAAAATCACACAATTCCA
AACCATGCTAAATGAGATTTAAAGAACAAACGATGGCAAAAGGCAA
CCGTTATAAATGTGATCTTTCTTGGCAGTTATCTGTCAATTTTTCT
AAGGAACAGTGAATTCATCATAGGAGAGATGTTATACGTTACATAA
TCATACATACTGCATGTATCTCACCTACTTTACCTCATCAACTCTA
AAACAGTTCTAGTCCCAACCCCAGATTCCTAGTCATGACACAAGTC
CGCACCGGACAGGACTCACAACCAGCAAGAGAAGCTAACAAATTTA
CGCCCCGGTAAAACATTCTTTAGGGGCCGTTCAATGGTAATTTTCC
TCTCACCCGTTTAAACTTACCTCCGGGCGGTATCTTCAATAACCTC
TGTTGTCCCCGGGTATCATTGGAAACAGTGAGGGACGTTGAACAGA
AGAGAGGATCACCGTAAATTTGCCTTGCAATTGGCCCTAACCACGG
ATGGTTAACTTCAAGCCATCACGACAGCAATTGAGTCGGCGCATAG
CTACCCTCCTCTTCTTGACCCCATGCATAGGACCAACCTTAACCGA
TGGAACAGGTTCCTCCGCTCCGTCCCCTGGTAGTGTCTCTGCGCAA
GAAATAGTTAAGGTATGAAGACTGATCTCTCGCACCCCCCTCACAG
TACTGTTATGGTGAATTGACAAAGCCATTGGCTAGATTGAAACATG
TAATTCATATGTAATCTTGTTCAATTAACGAGCTTCGTACAGTCTC
AATCTAGACGTCTGATAATGGCGTTTGTGCTCCTAATCGATGAGCC
ATCTCATGTGACGTCTATACGCTTCGATGGCTTCCGTCGCGAATAT
AGAACCACTTGAAATATGCTGCAAACCACGATCCACCCTGGTCCTG
AAAAGATATAAATACAGCACATCTAGCAGGCTTTTGTCTTCTTGGT
TGAAACACACAATTATAACAATCTACATCTAAAAATG
RPS7A (SEQ ID NO 139); RPPA04215 - Pichia
pastoris (IG-66), Contig1695_3185_3751
CCTATGTAAGCCTTCTCAGCATAGTCAACAAAGAAACCTGTTTCGA
CGACTCCTACCAGTCGTATCAGCTTTTCATCCAGCTCTTTAACCTT
ATTTAACGGAATCTCTCCAAAATCAGCATCAATGACGAAGTTACCA
TTATCTGTGACCACTGGACCAGCTTTCGCCTTTCCAGCATCTCTCA
GAGTGGCGCTGTTGGCTCCTAGTTTGAAAAGATCTTTCAATACTTT
AACATAACTGTTGGGTACAACCTCAATGGGAACGCCTTTGCGCCAG
AAATGACCCAGTCTTTCTGGAGACTGTTTACGAAAATCTGCCACCA
CAACGAATTTACGAGACAATGATGCCACCAACTTTTCTTGATAAAG
CGCTGCCCCTCCTCCCTTTATCAAATTCAAGTTGGTGTCAATTTCA
TCAGCTCCATCAAAGGCTACGTCTATCTCATCGAACTCCTCCACAT
TTCCTAAACGAAGGCCATTGTCCAAGATAAGTTGCTTTGATTGAAA
GCTGGTAGGGATACAAATAAACTTTTCTTTATTTTCCAACTGCCCC
AAGCGTTCGGCGACATAGACAACGGTGGATCCAGACCCAACTCCTA
TAATTCTGTCAGACTTCGAAACGTTTTCATTGACTGCTGCGAAAGC
AGCCAATTTTTTTGACTTCTCAATTAGTTCCTCAGACATTAGTTGT
CTAAGATGCTAGAGGCAAGACGTGAATAAGTACCCACAAAGGACGG
TCTAATTTCCCTTACATTCATCAATAGGCTATTTCAAGCATGAACT
TACATAAGCGTATTGTGCATTAGATATTAACCTCTTAGCATCCCAA
CACCGTACTGGGCTTCGACCCTAACCACATCCCCTACATCACATGA
CTGTAATTTTTTTTGTTACTACTCTTCTCTTGGGACTGTTGTGCGC
GTCGAAACTCACACGGTAACAGCAGTATCACAAGATG
RPL1 (SEQ ID NO 140); RPPA10390 - Pichia
pastoris (IG-66), Contig1965_1563_2216
CATAAGTCCTAGAACACCACTTGTTAGTAAAACCGGTAAAATTCCC
CATTTTCTCATATTCAACTTACAATGCTCAAAAGATGATCTTTGAA
GGCCATCCCAGTGGTGTTAGCCATCCTATGGAAGGGTTTGTTAGTA
ATTGCTGATCCAAAACCTGAAGACTGAAAAAAAGGCTCGCACATGT
CACTCATTACCACATCCACAGGGAATTGCTGCTTCTGCTTGTTACT
TATCTCGAGGTTATCGTCTACTTCAGCCTGAAAGTAGGAAGGTCCA
TCCGCTATTGGTTCAATTTCTTGAAGCTTTCTAAGGTCTTTGTCCA
TGTTCAACTGGTAAAAATGAGTCTTTATCATCTCATGCGTGGTCTT
ACTCAAGATGTTGGCCTGCATTGAAGACACCCCTTTTGGAGGCTCA
CAGGCTAGGATATCTATCCCAAGAATATTGCCTCTGGAAGTCACTT
TATCGGCTGCAACTTGGGACCATGCACCAGGAGCAAACCCCAAATC
CACGACATTTTGCCCTGGACGGAAGATATTGAACTTCTCATGGATT
TGGAGCAGCTTAAACGCTGCCCTTGATTTCAATGCCTTTGCGTGAG
CTTCCTTTGTGAAGGGGTCAGATGATTGACGTTCCAGCCATTTCCT
AGAGGATGAACTCTTTCCTTTTACTTTCAGGGAAACAAACCTAGCA
AACTGCCTCCGTAGCAGCGACACAGAAGGATGCTGTATCATGTGAT
GAACGTAGCTAAGCACAACCTGTGTGACAATTCCGTGTCGTTTATC
TTATCTTGCTAGTCTAGATATAAAGTATCACGTGACAAATATTATT
TTTGCCCCAACACCGGCTTGAAACTTAGGGCTCGCGCCCTCGTCGC
GAGAATTCTTACCTTTCGTTAAATTTTTTTTCAGTGTTCCAACTTT
TCCCTTCTCTATCCATCGTCGAATCAAACCTTTACGGGATTACCTA
CAACCTTTTTGCTAGGGAGACGAATTAATAGAAAATG
TKL1 (SEQ ID NO 141); RPPA06932 - Pichia
pastoris (IG-66), Contig1351_2056_46
GATATCGATTCCACTGCTCAGAGTCTTTTCATCAAGCTCTAAGAAA
AGTCCGGGGATGGAGGAGCCAGCGCAACAGAAAACTATGTCGGGAC
TAAAACCAACTTCCTCTAAAACACGCTCACACTGGTCATATTTGGA
GATATCTGCTGCCACGTAAGAAATTGTGTTGCCCTTCTCGGTCCCG
TGAATATCAATAGCGTCCTTTACTACCTGCTTTAGTAAGGATTCTG
TTCTAGCCACAATCACAACGGAACATCCTTTTCCATACAAAAGTTT
AGCAAACTCGGCTCCAACACCCTGGGATGCACCCGTAATAATTGCC
TTTTTGTTGGTGACATCAAAATTATCAAACATTCCAGTTCCCTCTA
CACTTTGTATGAGAATGATAGCTGAAATTGTGCACCAGATGTTAGA
AGATAAGGTCGTGTCATGAACTAATATCATGAATTCCGAGGGTGGC
TCAACAACTATTCACGTGACTTGGACGTTGGAAGTTGAGGTGGTTG
GTGGATGTTGCACGGAGTATCATTTGTAAGCATGAAATCAGTCTAA
AAAACTTGCAGAATAGCAGAGCGGTTCGGAAATTCATTCAAAACCA
CCTCCTCAGATTGGATCTGCCCTACTCTGTTTAGCTCTGGGAGATT
TTCTCGGTCGTGTTCTTTCGCTGGTCTACCCACGCTATAGGAATCG
CTGTGAACGCTACCTTCTTCCCAACTTCTCGGTGACTATTATAAGC
CATTCCCACTTTGTTTTCAAGCACCAACAACCCACCCCCACCTTAT
CTACTCCATCTTGGGTGTCCCCGCGCCTGTTGCAAAGTCCGAACCA
TAGAACCCCCGACCTTTGTCCCACTAACCCTCAGACACCCCTCGGA
AGTCAGGGAGAAACCACTCCGAAGTACATTAATCATCCCTCGTATT
CTCGACGGTGCCCATTTTCTTTATAAAAAGGGAGACACAGGTTGCT
TCACTAACTCTAGACTTGTATTCTACATCCACTCTACACAATG
PIS1 (SEQ ID NO 142); RPPA06550 - Pichia
pastoris (IG-66), Contig2109_25041_25727
TTTTTCCTCTTCGTTGTGTGGTAAACTCGGTAACGAGGCTAAAAGT
TTTTGCAAATTTGAGTCTTCATTCAAATCTAAGAACTCTGAATAAA
TCTCAAACACTTCTTTCATTTCGATCAATAAGTGTAGTAGTTTCTG
TCTATAAATTTCTTCGGTGTTTGTTCTTTCTAGAGCGTATTGTATA
CTAACAAAGTTGGATACGACCAAAGTTTGGAGCTCATTGTCCATTT
TGTGTAATCCTTCATCGTCATCAAGCAGAATTCTTAGCTCCTTTTG
GATCTTGGTTGTAAAAGGTCTCAACGTTGTAGTATTGTTCATAAGA
AGCTTCTGTAACACAAAATTAGCTGGCTTGGGGCAAAGATACATCA
CGCTAACAAGCGATTGAATAATTGACGGAAGCCTTGGTGTCAGTAT
TTCTCTGGTAAGTGCAACCTTATGCCTTATTTTGTCCATAATAAAA
TCTAGAGTGGATATAGCTGATTCCAAACAGGTCATCTGTCGTACAT
CAATGGTTGATGTGATGTAACACTTCGACTCAATAATCTTGGTCAA
AGCACTAATATAGTTACCAGCGTCCGATGCAATAATCCTTGGGTGA
AGGCAAAGGACGTGGACAAGTTTTGTGCCAAACCAACGGACTTCAG
ATTGATTTGATCTAAGATAATTTGCAGCACGAGAGGTAACATGCGT
CAAGTCGGCTTTTGAAGCTTCATTAACGGCGTTCTCATCAGCAAGG
ATGGGGAGTATTTCCATCAGATCCGCCTCAACTGAATTATCCTTCA
AATGCGGAATGACAACGTCGAGTCCAACAAATCCTTGATATCCCAT
CCTGGAAGTCCAATTGTATGATCTAAAGAGATTACTTCATTCTATT
TTTTTTTTTTTTTTATTGGAATTTTTCAGCCCAAGGCTCTCTCTAT
CAGTGTACCGACCAATACGTATCAGATACAGATAATTTTGAGCTCA
GGCAACATGGAAATTTACTTGTCTCTAGATAGTCATG
FET3 (SEQ ID NO 143); RPPA06678 - Pichia
pastoris (IG-66), Contig1928_10057_11658
TGGGCTTTCGTACCAAATGGAAAAATCACGTACAAGTATGCCCAGA
GCTAAGCTAATCGGATGGCAAGTAGAAAGTGGATGGGTTTCACAGA
ACAATAAAGAATGAGTGACGGATATTATTGGCTGGCAGGCATTAAA
GATGCATAATTTAAGCTTTCTGTTTTCTACTTTTGGAATTGTCACA
AATTTGAACTGTGGATGTTATTGAAACACAGACCCGTATAAATACC
TCTTGAGAGAAATTTGAAAGTGAAGCTATTTCAGTGAATTAATCAC
TCGCCATACACGAGGTAGATAATTCACGTAGACGAATTTCTTTTGA
TCCATTTTATTCAGGGTGGACAGTCAGAAGTGTTCGTTCACCTGAT
ATGTTCTAGATGCAGCTCGAAACGCTGTAAAAAAAAAAAGTCCCAA
AAGTCACGTGCATAAAGGTGTAGTTCAATTTAATGGAGATAACATA
ACATCTATGACTCCTTTCATGAATCCATCTCAAAAACACAAACTTT
GCTAGAATATCTGGTGGCACCGATTTTTCATCATTTCACGAGTTTA
TATAGCATATGCGCCAACAGAACGTTGCCTGACACAATGTTAAGGC
TTTTAAATTTTGCTTGTGTAGTAAAAAGTTAGTAGTGTGTTACTCG
ATATCATATTTCTATCAGAAGTGGAATATTCTAATCTCTCCTCTAC
CTTTGTTACAATCCGTTTCGAACAGAAAAAAGAATTTATGATGATT
TTATGGAGAATCATTCCAATAATAGCATTGCTAATTAGATTGACGG
TAGCAAAGACTCACAAGTTCAATTTGACAGCTTCTTGGGTGAAAGC
AAATCCAGATGGTGTTTTTGAGAGAGATGTCATTGGACTTAACGGA
CAGTGGCCTCTACCAGTTCTAAGGGTGAATCAAGGAGACAGGATCG
AACTGTTGTTGACGAATGGTCTTGGCAATGCGAATACATCTTTGCA
TTTCCACGGCCTGTTCCAAAGAGGATCTAGTTTTATG
FTR1 (SEQ ID NO 144); RPPA04329 - Pichia
pastoris (IG-66), Contig1928_8701_7604
TCGAGTAACACACTACTAACTTTTTACTACACAAGCAAAATTTAAA
AGCCTTAACATTGTGTCAGGCAACGTTCTGTTGGCGCATATGCTAT
ATAAACTCGTGAAATGATGAAAAATCGGTGCCACCAGATATTCTAG
CAAAGTTTGTGTTTTTGAGATGGATTCATGAAAGGAGTCATAGATG
TTATGTTATCTCCATTAAATTGAACTACACCTTTATGCACGTGACT
TTTGGGACTTTTTTTTTTTACAGCGTTTCGAGCTGCATCTAGAACA
TATCAGGTGAACGAACACTTCTGACTGTCCACCCTGAATAAAATGG
ATCAAAAGAAATTCGTCTACGTGAATTATCTACCTCGTGTATGGCG
AGTGATTAATTCACTGAAATAGCTTCACTTTCAAATTTCTCTCAAG
AGGTATTTATACGGGTCTGTGTTTCAATAACATCCACAGTTCAAAT
TTGTGACAATTCCAAAAGTAGAAAACAGAAAGCTTAAATTATGCAT
CTTTAATGCCTGCCAGCCAATAATATCCGTCACTCATTCTTTATTG
TTCTGTGAAACCCATCCACTTTCTACTTGCCATCCGATTAGCTTAG
CTCTGGGCATACTTGTACGTGATTTTTCCATTTGGTACGAAAGCCC
ACGAACCACACGACTGTACTTGACTTTTGGGGTCGTTAATGTGCAC
AGCCCAGAGATGATCTGATAATTAATATCATTTCGCACCACTGTTT
AAAAAATTCGATAATTTGTTACTAAAATGCTATTTTTGATGCAATG
CGTGCATGTTCATGCACCAGTAGATAATAATCTTAAATTTACAATA
TAGAAGCTAGTTTCTTAAAGTTTTATTGGCTTATGTGTTTTAGGGA
GAAAAGTTTCAGAGCTATATAAACTCGATCCTTCCTTCCACAAATC
TCGCCTTCAAGTCATTTCTCGAACTTCCTTCCAATAGCAGTGCCAC
TCAACGTTCAGTGGTCGTTGTAGTTCTTTTCAAAATG
NMT1 (SEQ ID NO 145); RPPA07685 - Pichia
pastoris (IG-66), Contig2096_16323_14941
TCGACACACAGAGGAAACGAGGGTAACATGATATCATGATGTACGT
ACAAACTAGGATCTCTGGTCATTCCACGGGCCACATCAGGATCGAA
TTTGTCGCCGTCCTCTTCTTCAGCGCCATCAGGGGCTCCAGCACCG
TCGTCGTATACGTAAACATCTAAAAAGGAGATATCGTCTTCAGTTC
TGGTGGAAAGGATCATATTGTCTGTGGGGTATACTTGCAACTCTTG
TTTCTCTTCTAAAATTTCACGTTCCGTTGGAAGAGTGATGTATGCG
TCACCATCTGCGTTGTTCTCATCTTCGTGGTAACGGGCACCCGTAC
TTGATAACCCAGGTAGCATCGAGACTTTGAGGCCATTGGCAACAGA
ATCCTCATACGTTTCGTCATCATCGTAATGTTCTAAATCATATTCC
TTTAGATCATCATCAATGTCATCTTGAGACTGTAGTTTGCTAGCTA
TAGATTTGTTGATGTTGGCTTTCATTTCTGAAATATCAGAATCATC
TTTCTGTGTTTCGGCCAGCTCATCCTGGGCATCTTTCAGTTTCAAA
TTGGCCATGGCTTCAATTCTCTCCATCTCAACATCGTTCATTTCAT
ATTTCTCGGGAAACTCCGAGGGGAAGCCTCTGGGAACCCATGTTGT
AGACGAAATCATAAGTGTACAATACTGTCTCTTTAAACTGAAAAAA
AAAATTTGAGAAGAAAAAAAAAATTGCTGGTCACATGATAAGGTGG
ATCCCGTAATCAAGCATTAACCTGGTTCAGGTTCGTGGATTGTTTA
GGCTTTATAAAATTGACTGGTAGGTCCCCAGTTTCAAGTTTTCTTT
AGTAATCTACTCGCACAAATTATTGTTGGGCATCTCGTCCTATCAG
ATTCATATAGATCTATAATTGATTCCCCTCCTGAAACACTTTCAAC
CAACATCTGAGGGTTCTCGAAAATTCAGTAAGATTGACTCCCTTCT
CATCCAGGCTTGGTGACCATCAAAATCACCAATCATG
PHO8 (SEQ ID NO 146); RPPA04253 - Pichia
pastoris (IG-66), Contig 1417_1294_2871
GGTATAAGTATAGCACATGTTGACGCTGATATAATCATCCCAAAAA
AGTGTGCGGTTTATTTAGAAAGGAGAAGAGGAACTCATGAAATTTT
TGAAGGAACGCACCCATTCTAGGTGATGTACGGGTGTCTTTTGATA
AGTAGTGGATAGGTTTTATTCACTGGACTATATGGTACTGTACAAA
AGAAGTTCCTCTTCAGATGGTCTGTAGCTTGCATGCAATGTTCTCT
CTAAGGAATGTGTATTTGAAAGGTCATGACTTACCATAACCAGTCG
CAAAATGCTTTTGTTCCAGGTTCTCTTCCTGAGGAACGTTTTGACG
GGGTATACATACCTTATCGAGATCAATTTTAGGAATAGATACTTTC
TCTCCAACCGTTAAAAATTTGGAAATCTTCACTTTGGCAGGTTTTA
ACTCGTCAAGAGACTTGACGTCTAGGATCTTATACTTGCTTATGTC
GTTCTTGGTGTCCTGATCGAGAGAGTCTTCCCTGACTTGGTATTTC
TTACCTTCGTGCTTAAAACTATGAGCCGTTTTGTCCTTAATGGGCA
TGGGAAGAGTTTTCAATGTGCTTAAATCCAAATCTTTGGGAGTTTT
CAACAGCCATACTTCTTTCTTTCCCAAATCCTTGAACGGAGATATT
TCTTTGAGGTCTGTGCGCTGTTTGAAATTAGCAGGTGGTTCAAAAG
TGAAATCCTCTGAATCACTGCTTTCAGAGTTGTCGGAGCTAATATC
GGACTCTGATATAAACTCCCTTGGATATTCTTTTCATTTCAGAGAC
ACCCTAATAGTTGTAGGAAAGATTAAACCAAGTGAAAAAAAAAATT
CAATCGCGATGGTAGAGTCTGGAAGGAGATGTTCTCCTTCTGGCTC
ACATCTCGCTAAGTGGACATTCTTCACGGAACTACGCACAAATCTA
TGCCCCTTTCAGGATCTCGCGCAATCTGCATTGATATGTGATATTT
GTTTTAGCGTCCTCTCCCCTTCAATTTCAAAGCAATG
FET3pre (SEQ ID NO 147); RPPA06003 - Pichia
pastoris (IG-66), Contig2108_1359_14
TTGTGGTCCTATGAATTAACCATTTAAAAATATTGCTATTAATGAT
TATGTTAGGTGGATTAATGTTGTCATTAGTACATATATTCGTATTT
TAACTTAATTACTCATTAATTAAGTGTAGAATGTCATTTCACTGGA
TTTAACTTGCTTTGTAAGTACGAAACAATGCTCGATCAATAATCAA
ACAGTTTAGTTCAGAGATTAGATTCAAATATAATTCTAAATTTATA
GTATCAGAAATATATATTAAGGACAGTGTTAATTTTACCTCATTTA
AACTGCTGCTAGCCATTAAACGATTGTGCTTTTTTAGTCCATGTTT
AAGAACAAATGACCATTTGCTAAACCTACTGTAATCTTATTATAAA
AATGAAGCCTACAAATCATTTCTTACAAGCAAATGATTTTACAGCC
CCTTGTGCTGAATGTATAGTACACATTGGACGAGGAGAAACGATCT
AAGTGGCCACTCGTAGGTGTTAATGATACTATTCGCTCGAACTTAA
CGGTGCTTAGGATTCTACTTAATCCTTGCACGTGACAAGGCTTAAA
TTAAGCTTGTTTTCTGATGTGTGTCAAAAAAATTTTAGCTTGACAT
ATATCGAACGATACGGCGTATTCTTAGGCAACAATTTCAACTTCTT
CGCTTATTTGCAATACTGGTATCCACGGGGTAATGCCAAACTTAAC
AAACAGTAATGGATTTGAAGCTAGCATGCTGTATTTCAAGGAGCGG
AGAAGCAAAGATAGCCCCCCTTAGATTAAATGCATGTTTAAGCTTT
CGAGGCACTTTTCTGATTAATGCATAAGCCTGCAGGAAAGGATTCC
ACTTATTTAGTAAAATTATCAGTTCAATCGCCAAATAAAGGCACAA
CAAGAAAAAGAAACAAAACAATAAAATTTCAACCTTCTTTCAGCGT
ATATAAAAGAAATTTACTGCCCACTTCCTCGAAGTTTTCCTTTCCC
CTAGCTGTAACCACTGCCGCCAACACTCCATCGAAGAATG

### REFERENCES

Archer, D., Jeenes, D. and Mackenzie, D. (1994). Strategies for improving heterologous protein production from filamentous fungi. Antonie Van Leeuwenhoek 65, 245-50.
Gasser, B., Maurer, M., Gach, J., Kunert, R. and Mattanovich, D. (2006). Engineering of Pichia pastoris for improved production of antibody fragments. Biotechnol Bioeng 94, 353-61.
Gething, M. and Sambrook, J. (1992). Protein folding in the cell. Nature 355, 33-45.
Hohenblum, H., et al. (2003) Assessing viability and cell-associated product of recombinant protein producing Pichia pastoris with flow cytometry. J Biotechnol 102, 281-290
Kurtzman C P. (2005). Description of Komagataella phaffii sp. nov. and the transfer of Pichia pseudopastoris to the methylotrophic yeast genus Komagataella. Int J Syst Evol Microbiol 55, 973-976.
Lang, C. and Looman, A. (1995). Efficient expression and secretion of Aspergillus niger RH5344 polygalacturonase in Saccharomyces cerevisiae. Appl Microbiol Biotechnol 44, 147-56.
Macauley-Patrick, S., Fazenda, M. L., McNeil, B. and Harvey, L. M. (2005). Heterologous protein production using the Pichia pastoris expression system. Yeast 22, 249-70.
Mattanovich, D., Gasser, B., Hohenblum, H. and Sauer, M. (2004). Stress in recombinant protein producing yeasts. J Biotechnol 113, 121-35.
Mori, K., Ogawa, N., Kawahara, T., Yanagi, H. and Yura, T. (2000). mRNA splicing-mediated C-terminal replacement of transcription factor Hac1p is required for efficient activation of the unfolded protein response. Proc Natl Acad Sci USA 97, 4660-5.
Porro, D., Sauer, M., Branduardi, P. and Mattanovich, D. (2005). Recombinant protein production in yeasts. Mol Biotechnol 31, 245-59.
Punt, P. J., van Biezen, N., Conesa, A., Albers, A., Mangnus, J. and van den Hondel, C. (2002). Filamentous fungi as cell factories for heterologous protein production. Trends Biotechnol 20, 200-6.
Sauer, M., Branduardi, P., Gasser, B., Valli, M., Maurer, M., Porro, D. and Mattanovich, D. (2004). Differential gene expression in recombinant Pichia pastoris analysed by heterologous DNA microarray hybridisation. Microb Cell Fact 3, 17.
Shuster, J. (1991). Gene expression in yeast: protein secretion. Curr Opin Biotechnol 2, 685-90.
Stryer, L. (1995). Biochemie. Spektrum der Wissenschaft Verlags GmbH.

We claim:

1. A method of increasing the secretion of a POI from a eukaryotic cell comprising:
providing a host cell comprising a recombinant nucleotide sequence encoding a POI and at least one recombinant nucleotide sequence encoding a protein that increases protein secretion; and
expressing in the host cell the recombinant nucleotide sequence encoding a POI and the at least one recombinant nucleotide sequence encoding a protein that increases protein secretion,
wherein said protein that increases protein secretion is selected from the group consisting of BMH2, BFR2, C0G6, C0Y1, CUP5, IMH1, KIN2, SEC31, SSA4, SSE1, and a biologically active fragment of any of the foregoing proteins.

2. The method according to claim 1, wherein the POI is a eukaryotic protein or a biologically active fragment thereof, preferably a Fab fragment, most preferably a Fab fragment of the monoclonal anti-HIV1 antibody 2F5.

3. The method according to claim 1 or 2, wherein the host cell is a fungal cell, preferably a yeast cell, or a higher eukaryotic cell, preferably a mammalian or a plant cell.

4. The method according to claim 3, wherein the yeast cell is a cell of the Komagataella genus, in particular a cell of a strain of Komagataella pastoris, Komagataella pseudopastoris or Komagataella phaffii.

5. The method according to any one of claims 1 to 4, wherein at least one recombinant nucleotide sequence encoding a protein that increases protein secretion is obtained from yeast, preferably from the species Saccharomyces cerevisiae or Pichia pastoris.

6. The method according to claim 5, wherein at least one recombinant nucleotide sequence encoding a protein that increases protein secretion is obtained from Saccharomyces cerevisiae and is identical with or corresponds to and has the functional characteristics of a sequence selected from the group consisting of SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40 and SEQ ID NO 41.

7. The method according to claim 5, wherein at least one recombinant nucleotide sequence encoding a protein that increases protein secretion is obtained from Pichia pastoris and is identical with or corresponds to and has the functional characteristics of a sequence selected from the group consisting of SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50 and SEQ ID NO 51.

8. The method according to any one of claims 1 to 7, wherein the recombinant nucleotide sequence encoding a POI is provided on a plasmid suitable for integration into the genome of the host cell or for autonomous replication in the host cell.

9. The method according to claim 8, wherein the plasmid is a eukaryotic expression vector, preferably a yeast expression vector.

10. The method according to claim 9, wherein the expression vector comprises a secretion leader sequence effective to cause secretion of the POI from the host cell.

11. The method according to claim 9 or 10, wherein the expression vector comprises a promoter sequence effective to control expression of the POI in the host cell.

12. The method according to any one of claims 1 to 11, wherein the nucleotide sequence encoding the POI is controlled by a promoter sequence which is a 1000 bp fragment from the 5′-non coding region of the PET9 gene of Pichia pastoris corresponding to SEQ ID NO 125, or a functionally equivalent variant thereof and the host cell is a cell of the genus Komagataella, in particular a cell of a strain of K. pastoris, K. pseudopastoris or K. phaffii.

13. Use of a nucleotide sequence isolated from Saccharomyces cerevisiae and encoding a protein that increases protein secretion and being selected from the group consisting of BMH2, BFR2, C0G6, C0Y1, CUP5, IMH1, KIN2, SEC31, SSA4, SSE1, and a biologically active fragment of any of the foregoing proteins, as a secretion enhancer, particularly as an enhancer of the secretion of a POI from a eukaryotic cell, preferably in a yeast cell and most preferred in a cell of a strain of K. pastoris, K. pseudopastoris or K. phaffii.

14. The use according to claim 13, wherein the nucleotide sequence encoding a protein that increases protein secretion is identical with or corresponds to and has the functional characteristics of a sequence selected from the group consisting of SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40 and SEQ ID NO 41.

15. Use of a nucleotide sequence isolated from Pichia pastoris and encoding a protein that increases protein secretion and being selected from the group consisting of BMH2, BFR2, C0G6, C0Y1, CUP5, IMH1, KIN2, SEC31, SSA4, SSE1, and a biologically active fragment of any of the foregoing proteins, as a secretion enhancer, particularly as an enhancer of the secretion of a POI from a eukaryotic cell, preferably in a yeast cell and most preferred in a cell of a strain of K. pastoris, K. pseudopastoris or K. phaffii.

16. The use according to claim 15, wherein the nucleotide sequence encoding a protein that increases protein secretion is identical with or corresponds to and has the functional characteristics of a sequence selected from the group consisting of SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50 and SEQ ID NO 51.

17. The use according to any one of claims 13 to 16 in a method according to claim 1.

18. A nucleotide sequence encoding a protein that increases protein secretion from a host cell, wherein the nucleotide sequence is isolated from Pichia pastoris and is identical with or corresponds to and has the functional characteristics of a sequence selected from the group consisting of a nucleotide sequence encoding the protein BMH2 (SEQ ID NO 42), a nucleotide sequence encoding the protein BFR2 (SEQ ID NO 43), a nucleotide sequence encoding the protein C0G6 (SEQ ID NO 44), a nucleotide sequence encoding the protein C0Y1 (SEQ ID NO 45), a nucleotide sequence encoding the protein CUP5 (SEQ ID NO 46), a nucleotide sequence encoding the protein IMH1 (SEQ ID NO 47), a nucleotide sequence encoding the protein KIN2 (SEQ ID NO 48), a nucleotide sequence encoding the protein SEC31 (SEQ ID NO 49), a nucleotide sequence encoding the protein SSA4 (SEQ ID NO 50) and a nucleotide sequence encoding the protein SSE1 (SEQ ID NO 51).

19. A yeast promoter sequence being a 1000 bp fragment from the 5′-non coding region of the PET9 gene corresponding to SEQ ID NO 125, or a functionally equivalent variant thereof and being isolated from Pichia pastoris.

20. The yeast promoter sequence of claim 19 which has, under comparable conditions, improved properties for expression of a POI in yeast, preferably in a strain of the genus Komagataella, in particular in a strain of Komagataella pastoris, Komagataella pseudopastoris or Komagataella phaffii, relative to a yeast promoter known in the art, in particular relative to a GAP promoter isolated from Pichia pastoris.

21. The yeast promoter sequence according to claim 20, having, under comparable conditions, at least the same, or at least about a 1.5-fold, or at least about a 2-fold, or at least about a 4-fold, 7-fold, 10-fold, or at least up to about a 15-fold promoter activity relative to a GAP promoter isolated from Pichia pastoris.

22. A eukaryotic expression vector based on the pPuzzle backbone further comprising the following components operably linked to each other:
a recombinant nucleotide sequence encoding a POI, optionally linked to a leader sequence effective to cause secretion of the POI from the host cell;
a promoter effective to control protein expression in a host cell;
a transcription terminator;
a selection marker;
either homologous integration sequences or autonomous replication sequences,
wherein the promoter is a 1000 bp fragment from the 5′-non coding region of the PET9 gene of Pichia pastoris (SEQ ID NO 125), or a functionally equivalent variant thereof, the transcription terminator is the transcription terminator of the cytochrome c gene from S. cerevisiae, the selection marker is a zeocin resistance gene and the host cell is a yeast cell, preferably a cell of a strain of the genus Komagataella, in particular a cell of a strain of Komagataella pastoris, Komagataella pseudopastoris or Komagataella phaffii.

23. Use of an expression vector as defined in claim 22 for recombinant expression of a POI in a host cell.

24. A yeast promoter sequence being isolated from Pichia pastoris and being identical with or corresponding to and having the functional characteristics of a sequence selected from the group consisting of a 1000 bp fragment from the 5′-non coding region of the GND1 gene (SEQ ID NO 126), a 1000 bp fragment from the 5′-non coding region of the GPM1 gene (SEQ ID NO 127), a 1000 bp fragment from the 5′-non coding region of the HSP90 gene (SEQ ID NO 128), a 1000 bp fragment from the 5′-non coding region of the KAR2 gene (SEQ ID NO 129), a 1000 bp fragment from the 5′-non coding region of the MCM1 gene (SEQ ID NO 130), a 1000 bp fragment from the 5′-non coding region of the RAD2 gene (SEQ ID NO 131), a 1000 bp fragment from the 5′-non coding region of the RPS2 gene (SEQ ID NO 132), a 1000 bp fragment from the 5′-non coding region of the RPS31 gene (SEQ ID NO 133), a 1000 bp fragment from the 5′-non coding region of the SSA1 gene (SEQ ID NO 134), a 1000 bp fragment from the 5′-non coding region of the THI3 gene (SEQ ID NO 135), a 1000 bp fragment from the 5′-non coding region of the TPI1 gene (SEQ ID NO 136), a 1000 bp fragment from the 5′-non coding region of the UBI4 gene (SEQ ID NO 137), a 1000 bp fragment from the 5′-non coding region of the ENO1 gene (SEQ ID NO 138), a 1000 bp fragment from the 5′-non coding region of the RPS7A gene (SEQ ID NO 139), a 1000 bp fragment from the 5′-non coding region of the RPL1 gene (SEQ ID NO 140), a 1000 bp fragment from the 5′-non coding region of the TKL1 gene (SEQ ID NO 141), a 1000 bp fragment from the 5′-non coding region of the PIS1 gene (SEQ ID NO 142), a 1000 bp fragment from the 5′-non coding region of the FET3 gene (SEQ ID NO 143), a 1000 bp fragment from the 5′-non coding region of the FTR1 gene (SEQ ID NO 144), a 1000 bp fragment from the 5′-non coding region of the NMT1 gene (SEQ ID NO 145), a 1000 bp fragment from the 5′-non coding region of the PHO8 gene (SEQ ID NO 146), and a 1000 bp fragment from the 5′-non coding region of the FET3 precursor (FET3pre) gene (SEQ ID NO 147), or a functionally equivalent variant of any of the foregoing sequences.

25. A eukaryotic expression vector based on the pPuzzle backbone further comprising the following components operably linked to each other:
a recombinant nucleotide sequence encoding a POI, optionally linked to a leader sequence effective to cause secretion of the POI from the host cell;
a promoter effective to control protein expression in a host cell;
a transcription terminator;
a selection marker;
either homologous integration sequences or autonomous replication sequences,
wherein the promoter is a yeast promoter sequence isolated from Pichia pastoris and is identical with or corresponds to and has the functional characteristics of a sequence selected from the group consisting of SEQ ID NO 125, SEQ ID NO 126, SEQ ID NO 127, SEQ ID NO 128, SEQ ID NO 129, SEQ ID NO 130, SEQ ID NO 131, SEQ ID NO 132, SEQ ID NO 133, SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 143, SEQ ID NO 144, SEQ ID NO 145, SEQ ID NO 146 and SEQ ID NO 147, or a functionally equivalent variant of any of the foregoing sequences, and the host cell is a yeast cell, preferably a cell of a strain of the genus Komagataella, in particular a cell of a strain of Komagataella pastoris, Komagataella pseudopastoris or Komagataella phaffii.

26. Use of an expression vector as defined in claim 25 for recombinant expression of a POI in a host cell.

27. Use of a yeast promoter sequence being isolated from Pichia pastoris and being identical with or corresponding to and having the functional characteristics of a sequence selected from the group consisting of SEQ ID NO 125, SEQ ID NO 126, SEQ ID NO 127, SEQ ID NO 128, SEQ ID NO 129, SEQ ID NO 130, SEQ ID NO 131, SEQ ID NO 132, SEQ ID NO 133, SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 143, SEQ ID NO 144, SEQ ID NO 145, SEQ ID NO 146 and SEQ ID NO 147, or a functionally equivalent variant of any of the foregoing sequences for modulation of the expression of a homologous POI in a host cell.

28. The use according to claim 27, wherein the yeast promoter sequence has an increased promoter activity relative to the native promoter sequence of the POI.

29. The use according to claim 27, wherein the yeast promoter sequence has a decreased promoter activity relative to the native promoter sequence of the POI.
